(19) United States
(12) Patent Application Publication
(10) Pub. No.: US 2023/0056230 A1
(43) Pub. Date: Feb. 23, 2023

(54) Therapy for the Treatment of Cancer

(71) Applicant: MacroGenics, Inc., Rockville, MD (US)

(72) Inventors: Bradley James Sumrow, Rockville, MD (US); Ross La Motte-Mohs, Rockville, MD (US); Jon Marc Wigginton, Rockville, MD (US); Ezio Bonvini, Rockville, MD (US); Paul A. Moore, Rockville, MD (US); Scott Koenig, Rockville, MD (US); Xiaoyu Zhang, Rockville, MD (US)

(73) Assignee: MacroGenics, Inc., Rockville, MD (US)

(21) Appl. No.: 17/787,793

(22) PCT Filed: Dec. 18, 2020
(86) PCT No.: PCT/US2020/065873

§ 371 Date: Jun. 21, 2022

Related U.S. Application Data
(60) Provisional application No. 63/123,581, filed on Dec. 10, 2020.
(60) Provisional application No. 63/031,453, filed on May 28, 2020.
(60) Provisional application No. 63/021,556, filed on May 7, 2020.
(60) Provisional application No. 63/019,857, filed on May 4, 2020.
(60) Provisional application No. 62/952,859, filed on Dec. 23, 2019.
(60) Provisional application No. 62/952,878, filed on Dec. 23, 2019.

(51) Int. Cl. C07K 16/28; A61P 35/00; C07K 16/32; A61K 45/06

(52) U.S. Cl. CPC C07K 16/2818; A61P 35/00; C07K 16/2803; C07K 16/2827; C07K 16/32; A61K 45/06; A61K 2039/505

(57) ABSTRACT

The present invention is directed to regimens for administering one or more Antibody-Based Molecules that bind PD-1 or PD-L1, and LAG-3 (e.g, a PD-1×LAG-3 bispecific molecule) alone, or in combination with an Antibody-Based Molecule that binds a Tumor Antigen (TA) for the treatment of cancer. The invention particularly concerns the use of such regimens in conjunction with PD-1×LAG-3 bispecific molecules. The invention is directed to the use of such molecules, and to the use of pharmaceutical compositions and pharmaceutical kits that contain such molecules and that facilitate the use of such dosing regimens in the treatment of cancer.

### CROSS-REFERENCE TO RELATED APPLICATIONS

This application claims priority to U.S. Patent Applications Ser. Nos. 63/123,581 (filed Dec. 10, 2020), 63/031,453 (filed May 28, 2020), 63/021,556 (filed May 7, 2020), 63/019,857 (filed May 4, 2020), 62/952,878 (filed Dec. 23, 2019), and 62/952,859 (filed Dec. 23, 2019), each of which applications is herein incorporated by reference in its entirety.

### REFERENCE TO SEQUENCE LISTING

This application includes one or more Sequence Listings pursuant to 37 C.F.R. 1.821 et seq., which are disclosed in computer-readable media (file name: 1301_0166PCT_ST25.txt, created on Dec. 18, 2020, and having a size of 70,042 bytes), which file is herein incorporated by reference in its entirety

### FIELD OF THE INVENTION

The present invention is directed to regimens for administering one or more Antibody-Based Molecules that bind PD-1 or PD-L1, and LAG-3 (e.g., a PD-1×LAG-3 bispecific molecule) alone, or in combination with an Antibody-Based Molecule that binds a Tumor Antigen (TA) for the treatment of cancer. The invention particularly concerns the use of such regimens in conjunction with PD-1×LAG-3 bispecific molecules. The invention is directed to the use of such molecules, and to the use of pharmaceutical compositions and pharmaceutical kits that contain such molecules and that facilitate the use of such dosing regimens in the treatment of cancer.

### BACKGROUND OF THE INVENTION

### I. Cell-Mediated Immune Responses

The ability of T-cells to optimally mediate an immune response against an antigen requires two distinct signaling interactions (Viglietta, V. et al. (2007) “Modulating Co-Stimulation,” Neurotherapeutics 4:666-675; Korman, A. J. et al. (2007) “Checkpoint Blockade in Cancer Immunotherapy,” Adv. Immunol. 90:297-339). First, antigen that has been arrayed on the surface of an Antigen-Presenting Cell (APC) must be presented to an antigen-specific naïve CD4⁺ T-cell. Such presentation delivers a signal via the T-Cell Receptor (TCR) that directs the T-cell to initiate an immune response that will be specific to the presented antigen. Second, a series of stimulatory and inhibitory signals, mediated through interactions between the APC and distinct T-cell surface molecules, triggers first the activation and proliferation of the T-cells and ultimately their inhibition. Thus, the first signal confers specificity to the immune response whereas the second signal serves to determine the nature, magnitude and duration of the response. The immune response is tightly controlled by co-stimulatory and co-inhibitory ligands and receptors often referred to as “immune checkpoints” (Chen et al., (2013) “Molecular Mechanisms of T Cell Co-Stimulation And Co-Inhibition,” Nature Rev. Immunol. 13:227-242; Pardoll, D. M., (2012) “The Blockade Of Immune Checkpoints In Cancer Immunotherapy,” Nat. Rev. Cancer 12(4):252-264). These molecules provide the second signal for T-cell activation and provide a balanced network of positive and negative signals which regulate immune responses to provide protection against infection and cancer. However, some cancer cells are able to escape the immune system by engendering a state of T-cell exhaustion in which T-cells are exposed to persistent antigen and/or inflammatory signals (Wherry E. J. (2010) “T Cell Exhaustion,” Nat. Immunol. 12(6):492-499). Two immune checkpoint molecules involved in T-cell exhaustion, Programmed Death-1 (“PD-1”) and Lymphocyte Activation Gene 3 (“LAG-3”) (Wherry, J. E. (2015) “Molecular And Cellular Insights Into T Cell Exhaustion,” Nat. Rev. Immunol. 15(8):486-499), are described in more detail below.

### II. Programmed Death-1 (“PD-1”)

Programmed Death-1 (“PD-1,” also known as “CD279”) is an immune checkpoint protein that is expressed on the surface of activated T-cells, B-cells and monocytes. It is an approximately 31 kD type I membrane protein member of the extended CD28/CTLA-4 family of T-cell regulators that broadly negatively regulates immune responses (Ishida, Y. et al. (1992) “Induced Expression Of PD-1, A Novel Member Of The Immunoglobulin Gene Superfamily, Upon Programmed Cell Death,” EMBO J. 11:3887-3895; US Patent Publication Nos. 2007/0202100; 2008/0311117; and 2009/00110667; U.S. Pat. Nos. 6,808,710; 7,101,550; 7,488,802; 7,635,757; and 7,722,868; PCT Publication No. WO 01/14557). PD-1 mediates its inhibition of the immune system by binding to the transmembrane protein ligands: Programmed Death-Ligand 1 (“PD-L1,” also known as “B7-H1”) and Programmed Death-Ligand 12 (“PD-L2,” also known as “B7-DC”) (Flies, D. B. et al. (2007) “The New B7s: Playing a Pivotal Role in Tumor Immunity,” J. Immunother. 30(3):251-260; U.S. Pat. Nos. 6,803,192 and 7,794,710; US Patent Publication Nos. 2005/0059051; 2009/0055944; and 2009/0274666; 2009/0313687; PCT Publication Nos. WO 01/39722 and WO 02/086083). In normal circumstances the immune checkpoint protein serves as the acting target for inhibiting the over-activation of T cells, and thus acts to prevent autoimmune damage. However, when its ligand is expressed by tumor cells, binding serves to prevent immune system cells from approaching the tumor, and thus weakens the ability of the immune system to recognize and destroy tumor cells (Tan, S. et al. (2020) “Cancer Immunotherapy: Pros, Cons And Beyond,” Biomed. Pharmacother. 124:109821:1-11). Accordingly, the overexpression of PD-L1 on tumor cells is often associated with poor prognosis.

The role of PD-1 ligand interactions in inhibiting T-cell activation and proliferation has suggested that these biomolecules might serve as therapeutic targets for treatments of inflammation and cancer. Thus, the use of antibodies to PD-1 and its ligand, particularly PD-L1 to treat infections and tumors and up-modulate an adaptive immune response has been proposed (see, Chocarro de Erauso, L. (2020) “Resistance to PD-L1/PD-1 Blockade Immunotherapy. A Tumor-Intrinsic or Tumor-Extrinsic Phenomenon?,” Front. Pharmacol. 11:441:1-13; Jiang, Y. et al. (2020) “Progress and Challenges in Precise Treatment of Tumors With PD-1/PD-L1 Blockade,” Front. Immunol. 11:339:1-7; Han, Y. et al. (2020) “PD-1/PD-L1 Pathway: Current Research In Cancer,” Am. J. Cancer Res. 10(3):727-742, US Patent Publication Nos. 2010/0040614; 2010/0028330; 2004/0241745; 2008/0311117; and 2009/0217401; U.S. Pat. Nos. 7,521,051; 7,563,869; and 7,595,048; PCT Publication Nos. WO 2004/056875 and WO 2008/083174). Antibodies capable of specifically binding to PD-1 and PD-L1 have been reported (see, e.g., Agata, T. et al. (1996) “Expression Of The PD-1 Antigen On The Surface Of Stimulated Mouse T And B Lymphocytes,” Int. Immunol. 8(5):765-772; and Berger, R. et al. (2008) “Phase I Safety And Pharmacokinetic Study Of CT-011, A Humanized Antibody Interacting With PD-1, In Patients With Advanced Hematologic Malignancies,” Clin. Cancer Res. 14(10):3044-3051; U.S. Pat. Nos. 8,008,449 and 8,552,154; US Patent Publication Nos. 2007/0166281; 2012/0114648; 2012/0114649; 2013/0017199; 2013/0230514 and 2014/0044738; and PCT Patent Publication Nos. WO 2003/099196; WO 2004/004771; WO 2004/056875; WO 2004/072286; WO 2006/121168; WO 2007/005874; WO 2008/083174; WO 2009/014708; WO 2009/073533; WO 2012/135408, WO 2012/145549; and WO 2013/014668).

### III. Lymphocyte Activation Gene 3 (“LAG-3”)

Lymphocyte Activation Gene 3 (“LAG-3,” also known as “CD223”) is a cell-surface receptor protein that is expressed by activated CD4⁺ and CD8⁺ T-cells and NK cells, and is constitutively expressed by plasmacytoid dendritic cells; LAG-3 is not expressed by B-cells, monocytes or any other cell types tested (Workman, C. J. et al. (2009) “LAG-3 Regulates Plasmacytoid Dendritic Cell Homeostasis,” J. Immunol. 182(4):1885-1891).

Studies have shown that LAG-3 plays an important role in negatively regulating T-cell proliferation, function and homeostasis and in T-cell exhaustion (Workman, C. J. et al. (2002) “Cutting Edge: Molecular Analysis Of The Negative Regulatory Function Of Lymphocyte Activation Gene-3,” J. Immunol. 169:5392-5395; Workman, C. J. et al. (2003) “The CD4-Related Molecule, LAG-3 (CD223) Regulates The Expansion Of Activated T-Cells,” Eur. J. Immunol. 33:970-979; Workman, C. J. (2005) “Negative Regulation Of T-Cell Homeostasis By Lymphocyte Activation Gene-3 (CD223),” J. Immunol. 174:688-695; Hannier, S. et al. (1998) “CD3/TCR Complex-Associated Lymphocyte Activation Gene-3 Molecules Inhibit CD3/TCR Signaling,” J. Immunol. 161:4058-4065, Blackburn, S. D., et al. (2009) “Coregulation of CD8+ T Cell Exhaustion By Multiple Inhibitory Receptors During Chronic Viral Infection” Nature Immunol. 10: 29-37).

Studies have suggested that inhibiting LAG-3 function through antibody blockade can reverse LAG-3-mediated immune system inhibition and partially restore effector function (Grosso, J. F. et al. (2009) “Functionally Distinct LAG-3 and PD-1 Subsets on Activated and Chronically Stimulated CD8 T-Cells,” J. Immunol. 182(11):6659-6669; Grosso, J. F. et al. (2007) “LAG-3 Regulates CD8⁺ T-Cell Accumulation And Effector Function During Self And Tumor Tolerance,” J. Clin. Invest. 117:3383-3392). Antibodies capable of specifically binding to LAG-3 have been reported (see, e.g., PCT Publication Nos. WO 2014/140180, WO 2015/138920, WO 2015/116539, WO 2016/028672, WO 2016/126858, WO 2016/200782 and WO 2017/015560).

### IV. Bispecific Molecules

The provision of bispecific molecules (e.g., bispecific antibodies, bispecific diabodies, etc.) provides a significant advantage over monospecific natural antibodies: the capacity to co-ligate and co-localize cells that express different epitopes. Bispecific molecules thus have wide-ranging applications including therapy. Bispecificity allows for great flexibility in the design and engineering in various applications, providing enhanced avidity to multimeric antigens, the cross-linking of differing antigens, and directed targeting to specific cell types relying on the presence of both target antigens. PD-1×LAG-3 bispecific molecules for use in the treatment of cancer and/or a disease associated with a pathogen are described in PCT Publication Nos. WO 2015/200119, WO 2017/025498, WO 2018/083087, WO 2018/185043, WO 2018/134279, and WO 2018/217940. In particular, PD-1×LAG-3 bispecific diabodies having novel PD-1- and LAG-3-Binding Domains and exemplary activity are described in WO 2017/019846.

### V. Tumor Antigens

Tumor Antigens (“TA”) comprise cell membrane proteins that are present only on tumor cells and not on any other cell (i.e., tumor-specific antigens) or are characteristically present on tumor cells, but are also present on certain normal cells (i.e., tumor-associated antigens). Tumor antigens can be targeted by antibodies and used to stimulate the cells of the immune system to overcome tumor escape, and play a renewed role in tumor surveillance and clearance (Tan, S. et al. (2020) “Cancer Immunotherapy: Pros, Cons And Beyond,” Biomed. Pharmacother. 124:109821:1-11; Finn, O. J. (2017) “Human Tumor Antigens Yesterday, Today, and Tomorrow,” Cancer Immunol. Res. 5(5):347-354; Barros, L. et al. (2018)“Immunological-Based Approaches For Cancer Therapy,” Clinics 73(suppl 1):e429s: 1-11; Smith, C. C. et al. (2019) “Alternative Tumour-Specific Antigens,” Nat. Rev. Cancer 19(8):465-478; Ehx, G. et al. (2019) “Discovery And Characterization Of Actionable Tumor Antigens,” Genome Med. 11:29:1-3).

### SUMMARY OF THE INVENTION

Provided herein are regimens capable of more vigorously directing the body's immune system to attack cancer cells. For although the adaptive immune system can be a potent defense mechanism against cancer and disease, it is often hampered by immune suppressive/evasion mechanisms in the tumor microenvironment, mediated by PD-1/PD-L1 interactions or by the inhibitory activity of LAG-3. As provided herein, such immune suppressive/evasion mechanisms can be overcome by the administration of a PD-1×LAG-3 bispecific molecule. As further provided herein, dual checkpoint inhibition of the PD/PD-L1 and LAG-3 checkpoint pathways can synergize with the anti-tumor activity of a TA-Binding Molecule (particularly one having enhanced ADCC activity).

The present invention is directed to regimens for administering one or more Antibody-Based Molecules that bind PD-1 or PD-L1, and LAG-3 (e.g., a PD-1×LAG-3 bispecific molecule alone, or in combination with an Antibody-Based Molecule that binds a Tumor Antigen (TA) for the treatment of cancer. The invention particularly concerns the use of such regimens in conjunction with PD-1×LAG-3 bispecific molecules. The invention is directed to the use of such molecules, and to the use of pharmaceutical compositions and pharmaceutical kits that that contain such molecules and that facilitate the use of such dosing regimens in the treatment of cancer.

The invention particularly concerns methods of treating a cancer comprising administering a PD-1×LAG-3 bispecific molecule to a subject in need thereof, wherein the method comprises administering the PD-1×LAG-3 bispecific molecule to the subject at a flat dose of from about 120 mg to about 800 mg.

The invention additionally concerns the embodiment of such a method, wherein the cancer is characterized by the expression of a Tumor Antigen (TA), and wherein the method further comprising administering to the subject a Tumor Antigen (TA) Binding Molecule (TA-Binding Molecule).

The invention further concerns methods of treating a cancer in a subject, wherein the cancer is characterized by the expression of a TA, the method comprising administering a TA-Binding Molecule to the subject and:
(a) a bispecific PD-1×LAG-3 bispecific molecule; or
(b) a molecule that immunospecifically binds PD-1 (PD-1-Binding Molecule) in combination with a molecule that immunospecifically binds LAG-3 (LAG-3-Binding Molecule); or
(c) a bispecific molecule that immunospecifically binds both PD-L1 and LAG-3 (PD-L1×LAG-3 bispecific molecule); or
(d) a molecule that immunospecifically binds PD-L1 (PD-L1-Binding Molecule) in combination with a LAG-3-Binding Molecule.

The invention additionally concerns the embodiment of the above described methods, wherein the TA-Binding Molecule comprises an ADCC-Enhanced Fc Domain.

The invention additionally concerns the embodiment of the above described methods, wherein:
(a) each molecule is in a separate composition; or
(b) each molecule is in the same composition; or
(c) the PD-1-Binding Molecule and the LAG-3-Binding Molecule are in the same composition, and the TA-binding molecule is in a separate composition; or
(d) the PD-L1-Binding Molecule and the LAG-3-Binding Molecule are in the same composition, and the TA-binding molecule is in a separate composition The invention additionally concerns the embodiment of the above described methods, wherein the TA-Binding Molecule is an antibody.

The invention additionally concerns the embodiment of the above described methods, wherein the PD-1-Binding Molecule is an antibody, the PD-L1-Binding Molecule is an antibody, and the LAG-3-Binding Molecule is an antibody.

The invention additionally concerns the embodiment of the above described methods, wherein the method comprises administering the TA-Binding Molecule and the PD-1×LAG-3 bispecific molecule.

The invention additionally concerns the embodiment of the above described methods, wherein the ADCC-Enhanced Fc Domain comprises:

(A) an engineered glycoform; and/or

(B) an amino acid substitution relative to a wild-type Fc Region.

The invention additionally concerns the embodiment of the above described methods, wherein the ADCC-Enhanced Fc Domain comprises:
(A) an engineered glycoform that is a complex N-glycoside-linked sugar chain that does not contain fucose, and/or that comprises a bisecting O-GlcNAc; and/or
(B) comprises an amino acid substitution is selected from the group consisting of:
   (a) one substitution selected from the group consisting of: F243L, R292P, Y300L, V305I, I332E, and P396L;
   (b) two substitutions selected from the group consisting of:
      (1) F243L and P396L;
      (2) F243L and R292P;
      (3) R292P and V305I; and
      (4) S239D and I332E;
   (c) three substitutions selected from the group consisting of:
      (1) F243L, R292P and Y300L;
      (2) F243L, R292P and V305I;
      (3) F243L, R292P and P396L; and
      (4) R292P, V305I and P396L;
   (d) four substitutions selected from the group consisting of:
      (1) F243L, R292P, Y300L and P396L; and
      (2) F243L, R292P, V305I and P396L; or
   (e) five substitutions selected from the group consisting of:
      (1) F243L, R292P, Y300L, V305I and P396L; and
      (2) L235V, F243L, R292P, Y300L and P396L,

wherein the numbering is that of the EU index as in Kabat.

The invention additionally concerns the embodiment of the above described methods, wherein the ADCC-Enhanced Fc Domain comprises the amino acid substitutions: L235V, F243L, R292P, Y300L and P396L, wherein the numbering is that of the EU index as in Kabat.

The invention additionally concerns the embodiment of the above described methods, wherein:
(A) the TA is selected from Table 6A or Table 6B; and/or
(B) the TA-Binding Molecule comprises the VL and VH Domains of an antibody selected from Table 7.

The invention additionally concerns the embodiment of the above described methods, wherein:
(A) the PD-1-Binding Molecule is an antibody that comprises:
   (a) a PD-1 VL Domain that comprises the amino acid sequence of SEQ ID NO:35, and a PD-1 VH Domain that comprises the amino acid sequence of SEQ ID NO:39;
   (b) a VH and VL Domain of an anti-PD-1 antibody selected from Table 1; or
   (c) a light chain and a heavy chain of an anti-PD-1 antibody selected from Table 1;
(B) the PD-L1-Binding Molecule is an antibody that comprises:
   (a) a PD-L1 VL Domain that comprises the amino acid sequence of SEQ ID NO:43, and a PD-L1 VH Domain that comprises the amino acid sequence of SEQ ID NO:47;
   (b) a VH and VL Domain of an anti-PD-L1 antibody selected from Table 2; or
   (c) a light chain and a heavy chain of an anti-PD-L1 antibody selected from Table 2; and
(C) the LAG-3-Binding Molecule is an antibody that comprises:
   (a) a LAG-3 VL Domain that comprises the amino acid sequence of SEQ ID NO:51, and a LAG-3 VH Domain that comprises the amino acid sequence of SEQ ID NO:55;
   (b) a VH and VL Domain of an anti-LAG-3 antibody selected from Table 3; or
   (c) a light chain and heavy chain of an anti-LAG-3 antibody selected from Table 3.

The invention additionally concerns the embodiment of the above described methods, wherein the PD-1×LAG-3 bispecific molecule comprises:
(a) a PD-1 VL Domain that comprises the amino acid sequence of SEQ ID NO:35, and a PD-1 VH Domain that comprises the amino acid sequence of SEQ ID NO:39, or a VH and VL Domain of an anti-PD-1 antibody selected from Table 1; and/or
(b) a LAG-3 VL Domain that comprises the amino acid sequence of SEQ ID NO:51, and a LAG-3 VH Domain that comprises the amino acid sequence of SEQ ID NO:55, or a VH and VL Doman of an anti-LAG-3 antibody selected from Table 3; or
(c) a bispecific Antibody-Based Molecule selected from Tables 4-5.

The invention additionally concerns the embodiment of the above described methods, wherein the PD-1×LAG-3 bispecific molecule comprises:

(a) two of the PD-1-Binding Domains; and

(b) two of the LAG-3-Binding Domains.

The invention additionally concerns the embodiment of the above described methods, wherein the PD-1×LAG-3 bispecific molecule comprises the PD-1 VL Domain of SEQ ID NO:35, the PD-1 VH Domain of SEQ ID NO:39, the LAG-3 VL Domain of SEQ ID NO:51, and the LAG-3 VH Domain of SEQ ID NO:55.

The invention additionally concerns the embodiment of the above described methods, wherein the PD-1×LAG-3 bispecific molecule or the PD-L1×LAG-3 bispecific molecule comprises an Fc Region and a Hinge Domain, and the embodiment, wherein the Fc Region and the Hinge Domain are both of the IgG4 isotype, and wherein the Hinge Domain comprises a stabilizing mutation.

The invention additionally concerns the embodiment of the above described methods, wherein the Fc Region is a variant Fc Region that comprises:
(a) one or more amino acid modifications that reduces the affinity of the variant Fc Region for an FcγR; and/or
(b) one or more amino acid modifications that enhances the serum half-life of the variant Fc Region.

The invention additionally concerns the embodiment of the above described methods, wherein the:
(a) modifications that reduce the affinity of the variant Fc Region for an FcγR comprise the substitution of L234A; L235A; or L234A and L235A; and
(b) modifications that enhances the serum half-life of the variant Fc Region comprise the substitution of M252Y; M252Y and S254T; M252Y and T256E; M252Y, S254T and T256E; or K288D and H435K,

wherein the numbering is that of the EU index as in Kabat.

The invention additionally concerns the embodiment of the above described methods, wherein the PD-1×LAG-3 bispecific molecule comprises two polypeptide chains of SEQ ID NO:59 and two polypeptide chains of SEQ ID NO:60.

The invention additionally concerns the embodiment of the above described methods, wherein the PD-1×LAG-3 bispecific molecule or the PD-L1×LAG-3 bispecific molecule is administered at a flat dose of about 300 mg, and the embodiment, wherein the PD-1×LAG-3 bispecific molecule or the PD-L1×LAG-3 bispecific molecule is administered at a flat dose of about 600 mg.

The invention additionally concerns the embodiment of the above described methods, wherein the flat dose is administered once about every 2 weeks, and the embodiment, wherein the flat dose is administered once about every 3 weeks.

The invention additionally concerns the embodiment of the above described methods, wherein the PD-1×LAG-3 bispecific molecule or the PD-L1×LAG-3 bispecific molecule is administered at a flat dose of about 600 mg once about every 2 weeks.

The invention additionally concerns the embodiment of the above described methods, wherein the PD-1×LAG-3 bispecific molecule or the PD-L1×LAG-3 bispecific molecule is administered at a flat dose of about 600 mg once about every 3 weeks.

The invention additionally concerns the embodiment of the above described methods, wherein the PD-1×LAG-3 bispecific molecule or the PD-L1×LAG-3 bispecific molecule is administered by intravenous (IV) infusion.

The invention additionally concerns the embodiment of the above described methods, wherein the cancer is selected from the group consisting of: adrenal gland cancer, AIDS-associated cancer, alveolar soft part sarcoma, anal cancer (including squamous cell carcinoma of the anal canal (SCAC)), bladder cancer, bone cancer, brain and spinal cord cancer, breast cancer (including, HER2⁺ breast cancer or Triple-Negative Breast Cancer (TNBC)), carotid body tumor, cervical cancer (including, HPV-related cervical cancer), chondrosarcoma, chordoma, chromophobe renal cell carcinoma, clear cell carcinoma, colon cancer, colorectal cancer, desmoplastic small round cell tumor, ependymoma, endometrial cancer (including, unselected endometrial cancer, MSI-high endometrial cancer, dMMR endometrial cancer, and/or POLE exonuclease domain mutation positive endometrial cancer), Ewing's sarcoma, extraskeletal myxoid chondrosarcoma, gallbladder or bile duct cancer (including, cholangiocarcinoma bile duct cancer), gastric cancer, gastroesophageal junction (GEJ) cancer, gestational trophoblastic disease, germ cell tumor, glioblastoma, head and neck cancer (including, squamous cell carcinoma of head and neck (SCCHN)), a hematological malignancy, a hepatocellular carcinoma, islet cell tumor, Kaposi's Sarcoma, kidney cancer, leukemia (including, acute myeloid leukemia), liposarcoma/malignant lipomatous tumor, liver cancer (including, hepatocellular carcinoma liver cancer (HCC)), lymphoma (including, diffuse large B-cell lymphoma (DLBCL), non-Hodgkin's lymphoma (NHL)), lung cancer (including, small cell lung cancer (SCLC), non-small cell lung cancer (NSCLC)), medulloblastoma, melanoma (including, uveal melanoma), meningioma, Merkel cell carcinoma, mesothelioma (including, mesothelial pharyngeal cancer), multiple endocrine neoplasia, multiple myeloma, myelodysplastic syndrome, neuroblastoma, neuroendocrine tumors, ovarian cancer, pancreatic cancer, papillary thyroid carcinoma, parathyroid tumor, pediatric cancer, peripheral nerve sheath tumor, pharyngeal cancer, pheochromocytoma, pituitary tumor, prostate cancer (including, metastatic castration resistant prostate cancer (mCRPC)), posterious uveal melanoma, renal metastatic cancer, rhabdoid tumor, rhabdomyosarcoma, sarcoma, skin cancer, a small round blue cell tumor of childhood (including neuroblastoma and rhabdomyosarcoma), soft-tissue sarcoma, squamous cell cancer, stomach cancer, synovial sarcoma, testicular cancer, thymic carcinoma, thymoma, thyroid cancer, and uterine cancer.

The invention additionally concerns the embodiment of the above described methods, wherein the cancer is selected from the group consisting of: anal cancer, breast cancer, bile duct cancer, cervical cancer, colorectal cancer, endometrial cancer, gastric cancer, GEJ cancer, head and neck cancer, liver cancer, lung cancer, lymphoma, melanoma, ovarian cancer and prostate cancer.

The invention additionally concerns the embodiment of the above described methods, wherein the cancer is selected from the group consisting of: HER2⁺ breast cancer, TNBC, cholangiocarcinoma bile duct cancer, HPV-related cervical cancer, SCCHN, HCC, SCLC or NSCLC, NHL, prostate cancer, gastric cancer and GEJ cancer.

The invention additionally concerns the embodiment of the above described methods, wherein the TA-Binding Molecule is a HER2-Binding Molecule comprising a HER2-Binding Domain comprising a Light Chain Variable Domain (VL_{HER2}) and a Heavy Chain Variable Domain (VH_{HER2}), wherein:
(A) the Light Chain Variable Domain (VL_{HER2}) comprises the Light Chain Variable Domain of margetuximab that comprises the CDR_{L}1, CDR_{L}2 and CDR_{L}3 of SEQ ID NO:61, and the Heavy Chain Variable Domain (VH_{HER2}) comprises the Heavy Chain Variable Domain of margetuximab that comprises the CDR_{H}1, CDR_{H}2 and CDR_{H}3 of SEQ ID NO:66;
(B) the Light Chain Variable Domain (VL_{HER2}) comprises the CDR_{L}1, CDR_{L}2 and CDR_{L}3 of trastuzumab and the Heavy Chain Variable Domain (VH_{HER2}) comprises the CDR_{H}1, CDR_{H}2 and CDR_{H}3 of trastuzumab;
(C) the Light Chain Variable Domain (VL_{HER2}) comprises the CDR_{L}1, CDR_{L}2 and CDR_{L}3 of pertuzumab and the Heavy Chain Variable Domain (VH_{HER2}) comprises the CDR_{H}1, CDR_{H}2 and CDR_{H}3 of pertuzumab; or
(D) the Light Chain Variable Domain (VL_{HER2}) comprises the CDR_{L}1, CDR_{L}2 and CDR_{L}3 of hHER2 MAB-1 and the Heavy Chain Variable Domain (VH_{HER2}) comprises the CDR_{H}1, CDR_{H}2 and CDR_{H}3 of hHER2 MAB-1.

The invention additionally concerns the embodiment of the above described methods, wherein the HER2-Binding Molecule is an anti-HER2 antibody.

The invention additionally concerns the embodiment of the above described methods, wherein the anti-HER2 antibody is margetuximab, and the method comprises administering margetuximab at a dosage of about 6 mg/kg to about 18 mg/kg once about every 3 weeks.

The invention additionally concerns the embodiment of the above described methods, wherein the method further comprises administered a chemotherapeutic agent.

The invention additionally concerns the embodiment of the above described methods, wherein the cancer is a HER2 expressing cancer, and particularly, wherein, wherein the HER2 expressing cancer is selected from the group consisting of: breast cancer, metastatic breast cancer, bladder, gastric cancer, GEJ cancer, ovarian cancer, pancreatic cancer, and stomach cancer.

The invention additionally concerns the embodiment of the above described methods, wherein the TA-Binding Molecule is a B7-H3-Binding Molecule comprising a B7-H3-Binding Domain comprising a Light Chain Variable Domain (VL) and a Heavy Chain Variable Domain (VH), wherein:
the VL comprises the CDR_{L}1, CDR_{L}2 and CDR_{L}3 of SEQ ID NO:71, and the VH the CDR_{H}1, CDR_{H}2 and CDR_{H}3 of SEQ ID NO:76.

The invention additionally concerns the embodiment of the above described methods, wherein the TA-Binding Molecule is enoblituzumab and the method comprises administering enoblituzumab at a dosage of about 6 mg/kg to about 18 mg/kg once about every 3 weeks.

The method of any one of claims 2-33 or 40-41, wherein the cancer is a B7-H3 expressing cancer, and particularly wherein the B7-H3 expressing cancer is selected from the group consisting of: anal cancer, SCAC, a breast cancer, TNBC, a head and neck cancer, SCCHN, lung cancer, NSCLC, melanoma, uveal melanoma, prostate cancer, and mCRPC.

The invention additionally concerns the embodiment of the above described methods, wherein the TA-binding molecule is administered by intravenous (IV) infusion.

The invention additionally concerns the embodiment of the above described methods, wherein cells expressing LAG-3 are present in a biopsy of the cancer prior to the treatment, and the embodiment, wherein cells expressing PD-1 are present in a biopsy of the cancer prior to the treatment.

The invention additionally concerns the embodiment of the above described methods, wherein co-expression of LAG-3 and PD-1 in a biopsy of the cancer prior to the treatment is indicative that such patient that is a candidate for such methods, and the embodiment, wherein expression is gene expression.

The invention additionally concerns the embodiment of the above described methods, wherein PD-L1 expression on the surface of cells of the cancer, prior to the treatment, is less than 1% as determined using a Combined Positive Score (CPS) or a Tumor Proportion Score (TPS).

### BRIEF DESCRIPTION OF THE DRAWINGS

FIG. 1 provide schematics showing representative covalently bonded tetravalent diabodies having four epitope-binding sites composed of two pairs of polypeptide chains (i.e., four polypeptide chains in all). One polypeptide of each pair has an E-coil Heterodimer-Promoting Domain and the other polypeptide of each pair has a K-coil Heterodimer-Promoting Domain. As shown, a cysteine residue may be present in a linker and/or in the Heterodimer-Promoting Domain. One polypeptide of each pair possesses a linker comprising a cysteine (which linker may comprise all or a portion of a hinge region) and CH2 and/or CH3 Domain, such that the associated chains form all or part of an Fc Region. VL and VH Domains that recognize the same epitope are shown using the same shading or fill pattern. In such embodiments wherein the two pairs of polypeptide chains are the same and the VL and VH Domains recognize different epitopes (as shown), the resulting molecule possesses four epitope-binding sites and is bispecific and bivalent with respect to each bound epitope. Alternatively, in such embodiments wherein the two pairs of polypeptides may be different and the VL and VH Domains of each pair of polypeptides recognize different epitopes, the resulting molecule possesses four epitope-binding sites and is tetraspecific and monovalent with respect to each bound epitope.

FIG. 2 shows observed and model-fitted PK profiles of the PD-1×LAG-3 bispecific molecule, DART-I, over a dose range of 1 mg to 1200 mg. Symbols represent observed data in individual patients and the solid lines represent model-fitted median curves for the dose group. The horizontal dashed line represents target threshold concentration based on clinical experience with other PD-1 targeting agents.

FIGS. 3A-3D plot the mean (SD) percent receptor occupancy (RO) of CD4+ cells (FIGS. 3A and 3C) and CD8+ cells (FIGS. 3B and 3D) by the PD-1×LAG-3 bispecific molecule, DART-I, on Day 1 of Cycle 1 (FIGS. 3A and 3B) or Cycle 2 (FIGS. 3C and 3D) at the end of administration of DART-I and prior to the administration of the next dose of that cycle. EOI=end of infusion after the administration of the first dose of Cycle 1 or Cycle 2. PRE=pre-dose prior to the administration of the next dose of Cycle 1 or Cycle 2. Missing error bars indicate N=1.

FIGS. 4A-4C show simulated multiple dose median PK profiles for administration of 400, 600, 800, 1000, and 1200 mg flat doses of the PD-1×LAG-3 bispecific molecule, DART-I, using Q2W (FIG. 4A), Q3W (FIG. 4B), and Q4W (FIG. 3C) regimens. The top horizontal dashed line represents target threshold trough concentration of 23 μg/mL based on clinical experience with other PD-1 targeting agents, the middle horizontal dashed line represents the RO EC₅₀×100, and the bottom horizontal dashed line represents the RO EC₅₀×10.

FIG. 5 presents a waterfall plot of the percent of reduction of target lesions (plotted as % change from baseline) among response-evaluable cohort expansion patients treated with the PD-1×LAG-3 bispecific molecule, DART-I, by tumor type.

FIGS. 6A-6E plot LAG-3 and PD-L1 scores from retrospective immunohistochemistry assays. Individual patient LAG-3 (FIG. 6A) and PD-L1 (FIG. 6B) scores from TNBC, EOC, and NSCLC cohorts are plotted order from high to low. Aggregate LAG-3 scores from TNBC, EOC, and NSCLC cohorts plotted by clinical response (FIG. 6C). Individual patient LAG-3 (FIG. 6D) scores from DLBCL cohort are plotted order from high to low, PD-L1 Scores are provided below. Aggregate LAG-3 scores from DLBCL cohort plotted by clinical response (FIG. 6E). PR=partial response; SD=stable disease; PD=progressive disease; CR=complete response.

FIG. 7 plots the gene expression of LAG-3 vs PD-1 (PDCD1) from retrospective NanoString PanCancer IO 360™ assays. Cancer types are indicated as follows: circle (•)=NSCLC; diamond (♦)=P-NSCLC; triangle (▴)=EOC; and square (▪)=TNBC. Clinical responses are indicated as follows: “R”=responder (partial response); “P”=progressive disease; “S”=stable disease; and symbol alone indicates unknown/undetermined.

FIG. 8 plots the IFN-7 Gene Signature scores from retrospective NanoString PanCancer IO 360™ assays by clinical response (PR—partial response; SD—stable disease; PD—progressive disease). Cancer types are indicated as follows: circle (•)=NSCLC; diamond (♦)=P-NSCLC; triangle (▴)=EOC; and square (▪)=TNBC.

FIG. 9 presents a comparison of the change in expression of checkpoint molecules on the surface of NK cells conditioned by exposure to TA-Binding Molecules having an ADCC-Enhanced Fc Domain or a wild-type Fc Domain. Flow cytometric analysis of the expression of CD137 (top row), LAG-3 (second row), PD-1 (third row), and PD-L1 (bottom row) on NK cells from PBMCs incubated with N87 HER2+ target cells in the presence of buffer (−), margetuximab (an anti-HER2 antibody having an ADCC-Enhanced Fc Domain), or rtrastuzumab (an anti-HER2 having a wild-type Fc Domain) each at 0.005 μg/ml or 0.05 μg/ml. The percent of positive cells (boxed) are indicated.

FIG. 10 shows the cytotoxicity of PMBCs pre-conditioned by exposure to TA-Binding Molecules having an ADCC-Enhanced Fc Domain or a wild-type Fc Domain. Plotted are the cytotoxicity curves toward K562 target cells mediated largely by NK cells pre-conditioned with margetuximab 0.005 μg/ml or 0.05 μg/ml (open and closed squares), rtrastuzumab 0.005 μg/ml or 0.05 μg/ml (open and close triangles) buffer (closed circles).

FIG. 11 presents a comparison of the change in expression of checkpoint molecules on the surface of NK cells, monocytes, CD4⁺, and CD8⁺ T cells conditioned by exposure to a TA-Binding Molecule having an ADCC-Enhanced Fc Domain. Flow cytometric analysis of the expression of LAG-3 (top row), PD-1 (second row), PD-L1 (third row), and CD137 (bottom row) on the different immune cell types present in PBMCs incubated with N87 HER2+ target cells in the presence of margetuximab (an anti-HER2 antibody having an ADCC-Enhanced Fc Domain), or a control antibody each at μg/ml or 0.5 μg/ml. The percent of positive cells (boxed) are indicated.

FIG. 12 shows the cytotoxicity of PBMCs pre-conditioned with TA-Binding Molecules having an ADCC-Enhanced Fc Domain (margetuximab) or a wild-type Fc Domain (rtrastuzumab) in the presence or absence of an anti-PD-1 antibody (retifanlimab) or a PD-1×LAG3 bispecific molecule (DART-I) toward K562 target cells (cytotoxicity largely mediated by NK cells).

FIG. 13 shows the cytotoxicity of PBMCs pre-conditioned with an ADCC-Enhanced TA-Binding Molecule (margetuximab) or a control in the presence or absence of a PD-1×LAG3 bispecific molecule (DART-I) toward K562(HER2 negative) or N87 (HER2⁺⁺⁺) target cells (cytotoxicity largely mediated by NK cells).

FIG. 14 shows a waterfall plot of the preliminary clinical results for 28 evaluable patients treated with the PD-1×LAG-3 bispecific molecule, DART-I, and the ADCC-Enhanced TA-Binding Molecule, margetuximab. The tumor types are indicated. Solid bars represent responses in patients receiving 600 mg DART-I+15 mg mg/kg; Striped bars represent responses in patients receiving 300 mg of DART-I+15 mg/kg.

FIGS. 15A-15C Plot the baseline gene expression of LAG3 and PD-1 (PDCD1) from 19 baseline biopsy samples in the cohorts treated with margetuximab and DART-I. Dual LAG3/PDCD1 expression at baseline is plotted in FIG. 15A. LAG-3 (FIG. 15B) and PDCD1 (FIG. 15C) expression at base line vs % change in target lesion(s) are plotted. CR=complete response; PR=partial response; SD=stable disease; PD=progressive disease.

### DETAILED DESCRIPTION OF THE INVENTION

The present invention is directed to regimens for administering one or more Antibody-Based Molecules that bind PD-1 or PD-L1, and LAG-3 (e.g., a PD-1×LAG-3 bispecific molecule alone, or in combination with an Antibody-Based Molecule that binds a Tumor Antigen (TA) for the treatment of cancer. The invention particularly concerns the use of such regimens in conjunction with PD-1×LAG-3 bispecific molecules. The invention is directed to the use of such molecules, and to the use of pharmaceutical compositions and pharmaceutical kits that that contain such molecules and that facilitate the use of such dosing regimens in the treatment of cancer.

I. Antibodies and Antibody-Based Molecules

An antibody is an immunoglobulin molecule that contains an Epitope-Binding Domain that is capable of immunospecific binding to a target region (“epitope”) of a molecule (such as an epitope of a tumor antigen (“TA”), an epitope of PD-1, an epitope of PD-L1, or an epitope of LAG-3), through at least one “Epitope-Binding Domain” located in the Variable Region of such immunoglobulin molecule. Such molecules may be of any isotype class (e.g., IgG, IgE, IgM, IgD, IgA and IgY), class (e.g., IgG₁, IgG₂, IgG₃, IgG₄, IgA₁ and IgA₂) or subclass.

As used herein, the terms “antibody” and “antibodies” are intended to include monoclonal antibodies, multispecific antibodies, human antibodies, humanized antibodies, synthetic antibodies, chimeric antibodies, polyclonal antibodies, and camelized antibodies. As used herein, the term “Antibody-Based Molecule” is intended to refer both to complete or intact antibody molecules and to molecules that are not complete or intact antibodies, but that comprise an Epitope-Binding Domain of an antibody (for example, single-chain Fvs (scFv), single-chain antibodies, Fab fragments, F(ab′) fragments, disulfide-linked bispecific Fvs (sdFv), intrabodies, diabodies, molecules that comprise the antibody's VL, VH or VL and VH Domains and molecules that comprise 1, 2 or 3 of the antibody's Light Chain CDR Domains, 1, 2 or 3 of the antibody's Heavy Chain CDR Domains, any 1, 2, 3, 4, or 5 of the antibody's Light Chain and Heavy Chain CDR Domains, or all 6 of the antibody's Light Chain and Heavy Chain CDR Domains). Such antibody-based molecules may be fusion proteins comprising additional components, e.g., peptide linkers, dimerization domains, etc.

The antibody-based molecules of the present invention are capable of “immunospecifically binding” to an epitope due to the presence of such epitope-binding domain(s). As used herein, an antibody or an epitope-binding fragment thereof is said to “immunospecifically” bind a region of another molecule (i.e., an epitope) if it reacts or associates more frequently, more rapidly, with greater duration and/or with greater affinity or avidity with that epitope relative to alternative epitopes (for example variant epitopes containing 1, 2, 3 or more than 3 amino acid substitutions, or polypeptides that possess less than 50% identity or that are unrelated). It is also understood by reading this definition that, for example, an Antibody-Based Molecule that immunospecifically binds a first target may or may not immunospecifically or preferentially bind to a second target. An epitope-containing molecule may have immunogenic activity, such that it elicits an antibody production response in an animal; such molecules are termed “antigens”.

Natural antibodies are capable of binding to only one epitope species (i.e., they are “monospecific”), although they can bind multiple copies of that species (i.e., exhibiting “bivalency” or “multivalency”). In this regard, the basic structural unit of a naturally occurring complete or intact IgG antibody is a tetramer composed of four assembled polypeptide chains: two shorter “Light Chains” complexed with two longer “Heavy Chains.” Each polypeptide chain is composed of an amino-terminal (“N-terminal”) portion that comprises a “Variable Domain” and a carboxy-terminal (“C-terminal”) portion that comprises at least one “Constant Domain.” An IgG Light Chain is composed of a single “Light Chain Variable Domain” (“VL”) and a single “Light Chain Constant Domain” (“CL”). Thus, the structure of the Light Chains of an IgG antibody is n-VL-CL-c (where n and c represent, respectively, the N-terminus and the C-terminus of the polypeptide chain). An IgG Heavy Chain is composed of a single “Heavy Chain Variable Domain” (“VH”), three “Heavy Chain Constant Domains” (“CH1,” “CH2” and “CH3”), and a “Hinge” Region (“H”), located between the CH1 and CH2 Domains. Unless specifically noted to the contrary, the order of domains of the protein molecules described herein is in the N-Terminal to C-Terminal direction. Thus, the structure of an IgG Heavy Chain is n-VH-CH1-H-CH2-CH3-c (where n and c represent, respectively, the N-terminus and the C-terminus of the polypeptide). The ability of an intact, unmodified antibody (e.g., an IgG antibody) to bind an epitope of an antigen depends upon the presence and sequences of the Variable Domains.

A. Constant Domains

### 1. Light Chain Constant Domain

One CL Domain is a human IgG CL Kappa Domain. The amino acid sequence of a representative human CL Kappa Domain is (SEQ ID NO:1):

RTVAAPSVFI FPPSDEQLKS GTASVVCLLN NFYPREAKVQ
WKVDNALQSG NSQESVTEQD SKDSTYSLSS TLTLSKADYE
KHKVYACEVT HQGLSSPVTK SFNRGEC

Another CL Domain is a human IgG CL Lambda Domain. The amino acid sequence of a representative human CL Lambda Domain is (SEQ ID NO:2):

QPKAAPSVTL FPPSSEELQA NKATLVCLIS
DFYPGAVTVA WKADSSPVKA GVETTPSKQS
NNKYAASSYL SLTPEQWKSH RSYSCQVTHE
GSTVEKTVAP TECS

### 2. Heavy Chain CH1 Domains

A representative CH1 Domain is a human IgG1 CH1 Domain. The amino acid sequence of a representative human IgG1 CH1 Domain is (SEQ ID NO:3):

ASTKGPSVFP LAPSSKSTSG GTAALGCLVK
DYFPEPVTVS WNSGALTSGV HTFPAVLQSS
GLYSLSSVVT VPSSSLGTQT YICNVNHKPS
NTKVDKRV

Another representative CH1 Domain is a human IgG2 CH1 Domain. The amino acid sequence of a representative human IgG2 CH1 Domain is (SEQ ID NO:4):

ASTKGPSVFP LAPCSRSTSE STAALGCLVK
DYFPEPVTVS WNSGALTSGV HTFPAVLQSS
GLYSLSSVVT VPSSNFGTQT YTCNVDHKPS
NTKVDKTV

Another representative CH1 Domain is a human IgG3 CH1 Domain. The amino acid sequence of a representative human IgG3 CH1 Domain is (SEQ ID NO:5):

ASTKGPSVFP LAPCSRSTSG GTAALGCLVK
DYFPEPVTVS WNSGALTSGV HTFPAVLQSS
GLYSLSSVVT VPSSSLGTQT YTCNVNHKPS
NTKVDKRV

Another representative CH1 Domain is a human IgG4 CH1 Domain. The amino acid sequence of a representative human IgG4 CH1 Domain is (SEQ ID NO:6):

ASTKGPSVFP LAPCSRSTSE STAALGCLVK
DYFPEPVTVS WNSGALTSGV HTFPAVLQSS
GLYSLSSVVT VPSSSLGTKT YTCNVDHKPS
NTKVDKRV

3. Heavy Chain Hinge Regions

A representative Hinge Region is a human IgG1 Hinge Region. The amino acid sequence of a representative human IgG1 Hinge Region is (SEQ ID NO:7):

EPKSCDKTHT CPPCP

Another representative Hinge Region is a human IgG2 Hinge Region. The amino acid sequence of a representative human IgG2 Hinge Region is (SEQ ID NO:8):

ERKCCVECPP CP

Another representative Hinge Region is a human IgG3 Hinge Region. The amino acid sequence of a representative human IgG3 Hinge Region is (SEQ ID NO:9):

ELKTPLGDTT HTCPRCPEPK SCDTPPPCPR
CPEPKSCDTP PPCPRCPEPK SCDTPPPCPR
CP

Another representative Hinge Region is a human IgG4 Hinge Region. The amino acid sequence of a representative human IgG4 Hinge Region is (SEQ ID NO:10):

ESKYGPPCPS CP

As described herein, an IgG4 Hinge Region may comprise a stabilizing mutation such as the S228P substitution (as numbered by the EU index as in Kabat). The amino acid sequence of a particular stabilized IgG4 Hinge Region is (SEQ ID NO:11):

ESKYGPPCPP CP

4. Heavy Chain CH2 and CH3 Domains and Fc Domains

The CH2 and CH3 Domains of the two Heavy Chains interact to form the “Fc Region” of IgG antibodies that is recognized by cellular Fc Receptors, including but not limited to Fc gamma Receptors (FcγRs). As used herein, the term “Fc Region” is used to define a C-terminal region of an IgG Heavy Chain. A portion of an Fc Region (including a portion that encompasses an entire Fc Region) is referred to herein as an “Fc Domain.” An Fc Domain is said to be of a particular IgG isotype, class or subclass if its amino acid sequence is most homologous to that isotype relative to other IgG isotypes, however hybrid Fc Domains comprising portions from different isotypes are contemplated.

The amino acid sequence of the CH2-CH3 domain of a representative human IgG1 is (SEQ ID NO:12):

```
231 240 250 260
APELLGGPSV FLFPPKPKDT LMISRTPEVT
              270 280 290
CVVVDVSHED PEVKFNWYVD GVEVHNAKTK
              300 310 320
PREEQYNSTY RVVSVLTVLH QDWLNGKEYK
              330 340 350
CKVSNKALPA PIEKTISKAK GQPREPQVYT
              360 370 380
LPPSREEMTK NQVSLTCLVK GFYPSDIAVE
              390 400 410
WESNGQPENN YKTTPPVLDS DGSFFLYSKL
              420 430 440
TVDKSRWQQG NVFSCSVMHE ALHNHYTQKS
        447
LSLSPGX
```

wherein, [symbol] is a lysine (K) or is absent.

[symbol]

The amino acid sequence of the CH2-CH3 Domain of a representative human IgG2 is (SEQ ID NO:13):

```
231 240 250 260
APPVA-GPSV FLFPPKPKDT LMISRTPEVT
              270 280 290
CVVVDVSHED PEVQFNWYVD GVEVHNAKTK
              300 310 320
PREEQFNSTF RVVSVLTVVH QDWLNGKEYK
              330 340 350
CKVSNKGLPA PIEKTISKTK GQPREPQVYT
              360 370 380
LPPSREEMTK NQVSLTCLVK GFYPSDISVE
              390 400 410
WESNGQPENN YKTTPPMLDS DGSFFLYSKL
              420 430 440
TVDKSRWQQG NVFSCSVMHE ALHNHYTQKS
        447
LSLSPGX
```

wherein X is a lysine (K) or is absent.

The amino acid sequence of the CH2-CH3 Domain of a representative human IgG3 is (SEQ ID NO:14):

```
231 240 250 260
APELLGGPSV FLFPPKPKDT LMISRTPEVT
              270 280 290
CVVVDVSHED PEVQFKWYVD GVEVHNAKTK
              300 310 320
PREEQYNSTF RVVSVLTVLH QDWLNGKEYK
              330 340 350
CKVSNKALPA PIEKTISKTK GQPREPQVYT
              360 370 380
LPPSREEMTK NQVSLTCLVK GFYPSDIAVE
              390 400 410
WESSGQPENN YNTTPPMLDS DGSFFLYSKL
              420 430 440
TVDKSRWQQG NIFSCSVMHE ALHNRFTQKS
        447
LSLSPGX
```

wherein, X is a lysine (K) or is absent.

The amino acid sequence of the CH2-CH3 Domain of a representative human IgG4 is (SEQ ID NO:15):

```
231 240 250 260
APEFLGGPSV FLFPPKPKDT LMISRTPEVT
              270 280 290
CVVVDVSQED PEVQFNWYVD GVEVHNAKTK
              300 310 320
PREEQFNSTY RVVSVLTVLH QDWLNGKEYK
              330 340 350
CKVSNKGLPS SIEKTISKAK GQPREPQVYT
              360 370 380
LPPSQEEMTK NQVSLTCLVK GFYPSDIAVE
              390 400 410
WESNGQPENN YKTTPPVLDS DGSFFLYSRL
              420 430 440
TVDKSRWQEG NVFSCSVMHE ALHNHYTQKS
        447
LSLSLGX
```

wherein, [symbol] is a lysine (K) or is absent.

Throughout the present specification, the numbering of the residues in the constant regions of an IgG heavy chain is that of the EU index as in Kabat et al., SEQUENCES OF PROTEINS OF IMMUNOLOGICAL INTEREST, 5h Ed. Public Health Service, NH1, MD (1991), expressly incorporated herein by references. The term “EU index as in Kabat” refers to the numbering of the human IgG1 EU antibody.

Polymorphisms have been observed at a number of different positions within antibody constant regions (e.g., CH1 positions, including but not limited to positions 192, 193, and 214; Fc positions, including but not limited to positions 270, 272, 312, 315, 356, and 358 as numbered by the EU index as in Kabat), and thus slight differences between the presented sequence and sequences in the prior art can exist. Polymorphic forms of human immunoglobulins have been well-characterized. At present, 18 Gm allotypes are known: G1m (1, 2, 3, 17) or G1m (a, x, f, z), G2m (23) or G2m (n), G3m (5, 6, 10, 11, 13, 14, 15, 16, 21, 24, 26, 27, 28) or G3m (b1, c3, b3, b0, b3, b4, s, t, g1, c5, u, v, g5) (Lefranc, et al., “The Human IgG Subclasses: Molecular Analysis Of Structure, Function And Regulation.” Pergamon, Oxford, pp. 43-78 (1990); Lefranc, G. et al., 1979, Hum. Genet.: 50, 199-211). It is specifically contemplated that the antibodies of the present invention may be incorporate any allotype, isoallotype, or haplotype of any immunoglobulin gene, and are not limited to the allotype, isoallotype or haplotype of the sequences provided herein. Furthermore, in some expression systems the C-terminal amino acid residue (bolded above) of the CH3 Domain may be post-translationally removed. Accordingly, the C-terminal residue of the CH3 Domain is an optional amino acid residue in the molecules of the invention. Specifically encompassed by the instant invention are molecules lacking the C-terminal residue of the CH3 Domain. Also specifically encompassed by the instant invention are such molecules comprising the C-terminal lysine residue of the CH3 Domain.

The Fc Domain of the Fc Domain-containing Antibody-Based Molecules of the present invention may be either a complete Fc Domain (e.g., a complete IgG Fc Region) or only a portion of an Fc Region. Optionally, the Fc Domain of the Fc Domain-containing molecules of the present invention lacks the C-terminal lysine amino acid residue of wild-type IgG CH3 Domains.

In traditional immune function, the interaction of antibody-antigen complexes with cells of the immune system results in a wide array of responses, ranging from effector functions such as antibody dependent cytotoxicity, mast cell degranulation, and phagocytosis to immunomodulatory signals such as regulating lymphocyte proliferation and antibody secretion. All of these interactions are initiated through the binding of the Fc Domain of antibodies or immune complexes to specialized cell-surface receptors on hematopoietic cells. The diversity of cellular responses triggered by antibodies and immune complexes results from the structural heterogeneity of the three Fc receptors: FcγRI (CD64), FcγRII (CD32), and FcγRIII (CD16). FcγRI (CD64), FcγRIIA (CD32A) and FcγRIII (CD16) are activating (i.e., immune system enhancing) receptors; FcγRIIB (CD32B) is an inhibiting (i.e., immune system dampening) receptor. In addition, interaction with the Neonatal Fc Receptor (FcRn) mediates the recycling of IgG molecules from the endosome to the cell surface and release into the blood. The amino acid sequence of the CH2-CH3 Domains of representative wild-type IgG1 (SEQ ID NO:12), IgG2 (SEQ ID NO:13), IgG3 (SEQ ID NO:14), and IgG4 (SEQ ID NO:15) are presented above.

The amino acid sequence of the Fc Domain may be modified in order to provide an altered phenotype, for example an altered serum half-life, altered stability, altered susceptibility to cellular enzymes, altered effector function, or a combination of such phenotypes. In particular, the present invention contemplates Antibody-Based Molecules that comprise a wild-type Fc Domain, or an Fc Domain that has been modified to enhance its ability mediate Antibody-Dependent Cellular Cytotoxicity (ADCC) relative to the ADCC mediated by such Antibody-Based Molecule containing an Fc Domain without such modification. Such modified Fc Domains are referred to herein as “ADCC-Enhanced Fc Domains.” The present invention also contemplates Antibody-Based Molecules that comprise Fc Domain having little or no ADCC activity. Accordingly, in certain embodiments, the Antibody-Based Molecules of the present invention may be engineered to comprise an ADCC-Enhanced Fc Domain, or an Fc Domain having little or no ADCC activity. Although the Fc Domain of the Antibody-Based Molecules of the present invention may possess the ability to bind to one or more Fc receptors (e.g., FcγR(s)), in certain embodiments, such Fc Domains are modified Fc Domains having altered binding to FcγRIA (CD64), Fc-RIIA (CD32A), FcγRIIB (CD32B), FcγRIIIA (CD16a) or FcγRIIIB (CD16b)(relative to the binding exhibited by an Fc Domain without such modification). For example such variant Fc Domains may have enhanced binding to activating receptor(s) and/or will have substantially reduced or no ability to bind to inhibitory receptor(s) and will exhibit enhanced ADCC activity. Alternatively, such variant Fc Domains may have substantially reduced or no ability to bind activating receptor(s) and/or will have enhanced binding to inhibitory receptor(s) and will exhibit little or no ADCC activity.

Modifications that reduce or eliminate FcγR binding (and ADCC activity) are well-known in the art and include amino acid substitutions at positions 234 and 235, a substitution at position 265 or a substitution at position 297, as numbered by the EU index as in Kabat (see, for example, U.S. Pat. No. 5,624,821). In one embodiment, the Antibody-Based Molecules of the present invention comprise an Fc Domain having little or no ADCC activity that comprises 1, 2, 3, or 4 of the substitutions: L234A, L235A, D265A, N297Q, and N297G. In a specific embodiment the Antibody-Based Molecules of the present invention comprise an Fc Domain having little or no ADCC activity that comprise a substitution at position 234 with alanine and a substitution at position 235 with alanine (234A, 235A), as numbered by the EU index as in Kabat. Alternatively, such molecules may comprise a naturally occurring Fc Domain that inherently exhibits decreased (or substantially no) binding to FcγRIIIA (CD16a) and/or reduced effector function (relative to the binding and effector function exhibited by a wild-type IgG1 Fc Domain). In a specific embodiment, the Fc-bearing molecules of the present invention comprise an IgG2 Fc Domain (SEQ ID NO:13) or an IgG4 Fc Domain (SEQ ID:NO:15). When an IgG4 Fc Domain is utilized, the instant invention also encompasses the introduction of a stabilizing mutation, such as the Hinge Region S228P substitution described above (see, e.g., SEQ ID NO:11).

The, ADCC-Enhanced Fc Domains of the present invention may include some or all of the CH2 Domain and/or some or all of the CH3 Domain of a complete Fc Domain, or may comprise a variant CH2 and/or a variant CH3 sequence (that may include, for example, one or more substitutions and/or insertions and/or one or more deletions with respect to the CH2 or CH3 Domains of a complete Fc Domain). Such Fc Domains may comprise non-Fc polypeptide portions, or may comprise portions of non-naturally complete Fc Domains, or may comprise non-naturally occurring orientations of CH2 and/or CH3 Domains (such as, for example, two CH2 Domains or two CH3 Domains, or in the N-Terminal to C-Terminal direction, a CH3 Domain linked to a CH2 Domain, etc.).

ADCC-Enhanced Fc Domains identified as altering effector function (e.g., ADCC) are known in the art, including modifications that increase binding to activating Fc receptors (e.g., FcγRIIA (CD16A)) relative to inhibitory Fc receptors (e.g., FcγRIIB (CD32B)) (see, e.g., Stavenhagen, J. B. et al. (2007) “Fc Optimization Of Therapeutic Antibodies Enhances Their Ability To Kill Tumor Cells In Vitro And Controls Tumor Expansion In Vivo Via Low-Affinity Activating Fcgamma Receptors,” Cancer Res. 57(18):8882-8890)). Numerous single, double, triple, quadruple, and quintuple substitutions that enhance ADCC activity have been described (see, e.g., U.S. Pat. Nos. 6,737,056; 7,317,091; 7,355,008; 7,960,512; 8,217,147; 8,652,466).

In one embodiment, ADCC-Enhanced Fc Domains comprise an Fc Domain that comprises one or more amino acid substitutions (relative to a wild-type IgG Fc Domain) selected from: S239D, F243L, D270E, R292G, R292P, Y300L, V305I, I332E or P396L substitutions, as numbered by the EU index as in Kabat. These amino acid substitutions may be present in a human IgG Fc Domain (e.g., IgG1 Fc Domain) in any combination. In one embodiment, the variant human IgG Fc Domain contains an S239D and I332E substitution. In another embodiment, the variant human IgG Fc Domain contains a F243L, R292P and Y300L substitution. In a further embodiment, the variant human IgG Fc Domain contains a F243L, R292P, Y300L, V305I and P296L substitution. In a specific embodiment, such human IgG ADCC-Enhanced Fc Domain will comprise:

(a) one substitution selected from the group consisting of:
(1) F243L;
(2) R292P;
(3) Y300L;
(4) V305I;
(5) I332E; and
(6) P396L

(b) two substitutions selected from the group consisting of:
(1) F243L and P396L;
(2) F243L and R292P;
(3) R292P and V305I; and
(4) S239D and I332E

(c) three substitutions selected from the group consisting of:
(1) F243L, R292P and Y300L;
(2) F243L, R292P and V305I;
(3) F243L, R292P and P396L; and
(4) R292P, V305I and P396L;

(d) four substitutions selected from the group consisting of:
(1) F243L, R292P, Y300L and P396L; and
(2) F243L, R292P, V305I and P396L; or

(e) five substitutions selected from the group consisting of:
(1) F243L, R292P, Y300L, V305I and P396L; and
(2) L235V, F243L, R292P, Y300L and P396L,

wherein the numbering is that of the EU index as in Kabat.

In specific embodiments, an ADCC-Enhanced Fc Domain will comprise:
(1) an “FcMT1” ADCC-Enhanced Fc Domain, wherein such a Domain comprises F243L, R292P, Y300L, V305I, and P396L substitutions. Antibody-Based Molecules that comprise an FcMT1 variant IgG1 Fc Domain exhibit a 10-fold increase in binding to human CD16A (FcγRIIIA) relative to the binding observed with a wild-type IgG1 Fc Domain, and binding to CD16-158Phe is enhanced in a proportionally greater fashion than binding to CD16-158Val. The amino acid sequence of an “FcMT1” ADCC-Enhanced Fc Domain is (SEQ ID NO:16):

APELLVGPSV FLLPPKPKDT LMISRTPEVT
CVVVDVSHED PEVKFNWYVD GVEVHNAKTK
PPEEQYNSTL RVVSILTVLH QDWLNGKEYK
CKVSNKALPA PIEKTISKAK GQPREPQVYT
LPPSRDELTK NQVSLTCLVK GFYPSDIAVE
WESNGQPENN YKTTPLVLDS DGSFFLYSKL
TVDKSRWQQG NVFSCSVMHE ALHNHYTQKS
LSLSPGX

wherein, X is a lysine (K) or is absent
(2) an “FcMT2” ADCC-Enhanced Fc Domain, wherein such a Domain comprises L235V, F243L, R292P, Y300L, and P396L substitutions. The FcMT2 variant IgG1 Fc Domain is a further refinement of the FcMT1 variant IgG1 Fc Domain, and has similar CD16A binding properties, but has a more favorable reduction in binding to CD32B (FcγRIIB). The amino acid sequence of an “FcMT2” ADCC-Enhanced Fc Domain is (SEQ ID NO:17):

APELVGGPSV FLLPPKPKDT LMISRTPEVT CVVVDVSHED
PEVKFNWYVD GVEVHNAKTK PPEEQYNSTL RVVSVLTVLH
QDWLNGKEYK CKVSNKALPA PIEKTISKAK GQPREPQVYT
LPPSRDELTK NQVSLTCLVK GFYPSDIAVE WESNGQPENN
YKTTPLVLDS DGSFFLYSKL TVDKSRWQQG NVFSCSVMHE
ALHNHYTQKS LSLSPGX

wherein, X is a lysine (K) or is absent or
(3) an “FcMT3” ADCC-Enhanced Fc Domain, wherein such a Domain comprises F243L, R292P, and Y300L substitutions. The FcMT3 variant IgG1 Fc Domain is a further refinement of the FcMT1 variant IgG1 Fc Domain, and has similar CD16A binding properties, but has a more favorable reduction in binding to CD32B (FcγRIIB). The amino acid sequence of an “FcMT3” ADCC-Enhanced Fc Domain is (SEQ ID NO:18):

APELLGGPSV FL [symbol] PPKPKDT LMISRTPEVT CVVVDVSHED
PEVKFNWYVD GVEVHNAKTK P [symbol] EEQYNST [symbol] RVVSVLTVLH
QDWLNGKEYK CKVSNKALPA PIEKTISKAK GQPREPQVYT
LPPSRDELTK NQVSLTCLVK GFYPSDIAVE WESNGQPENN
YKTTPPVLDS DGSFFLYSKL TVDKSRWQQG NVFSCSVMHE
ALHNHYTQKS LSLSPG

[symbol]

wherein, X is a lysine (K) or is absent

In alternative embodiments, ADCC-Enhanced Fc Domains comprise an engineered glycoform that is a complex N-glycoside-linked sugar chain that does not contain fucose, and/or comprises a bisecting O-GlcNAc. Such glycoforms can be obtained by expressing the Antibody-Based Molecule recombinantly in cell lines that lack fucosylatransferase (e.g., POTELLIGENT® cell lines, BioWa, Inc.; Matsushita, T. (2011) “Engineered Therapeutic Antibodies With Enhanced Effector Functions: Clinical Application Of The Potelligent® Technology,” Korean J. Hematol. 46(3):148-150), and/or in cell lines that express O-GlcNAc transferase (Roche GlycArt AG; Satoh, M. et al. (2006) “Non-Fucosylated Therapeutic Antibodies As Next-Generation Therapeutic Antibodies,” Exp. Opin. Biol. Ther. 6(11):1161-1173). In certain embodiments, ADCC-Enhanced Fc Domains comprise comprises one or more amino acid substitutions and an engineered glycoform.

Additionally, the serum half-life of molecules comprising an Fc Domain may be increased by increasing the binding affinity of the Fc Domain for FcRn. The term “half-life” as used herein means a pharmacokinetic property of a molecule that is a measure of the mean survival time of the molecules following their administration. Half-life can be expressed as the time required to eliminate fifty percent (50%) of a known quantity of the molecule from a subject's body (e.g., a human patient or other mammal) or a specific compartment thereof, for example, as measured in serum, i.e., circulating half-life, or in other tissues. In general, an increase in half-life results in an increase in mean residence time (MRT) in circulation for the molecule administered. Modifications capable of increasing the half-life of an Fc Domain-containing molecule are known in the art and include, for example amino acid substitutions M252Y, S254T, T256E, and combinations thereof. For example, see the modifications described in U.S. Pat. Nos. 6,277,375, 7,083,784; 7,217,797, and 8,088,376; US Publication Nos. 2002/0147311; and 2007/0148164; and PCT Publication Nos. WO 98/23289; WO 2009/058492; and WO 2010/033279).

In one embodiment, Antibody-Based Molecules of the present invention comprise a variant Fc Domain, wherein such variant Fc Domain comprises a substitution at position 252 with tyrosine, 254 with threonine, and 256 with glutamate (252Y, 254T and 256E), as numbered by the EU index as in Kabat.

The present invention also encompasses Antibody-Based Molecules of the present invention comprising an Fc Domain wherein such Fc Domain comprises:
(a) one or more mutations which alter effector function and/or FcγR binding; and/or
(b) one or more mutations which extend serum half-life.

In one embodiment, the Antibody-Based Molecules of the invention comprise an Fc Domain wherein such Fc Domain comprises:

(a) one or more mutations which reduced or eliminates ADCC; and/or

(b) one or more mutations which extend serum half-life.

A representative IgG1 sequence for the CH2 and CH3 Domains of a variant Fc Domain having little or no ADCC activity and extended serum half-life comprises the substitutions L234A/L235A/M252Y/S254T/T256E (SEQ ID NO:19):

APE [symbol] GGPSV FLFPPKPKDT L [symbol] I [symbol] R [symbol] PEVT CVVVDVSHED
PEVKFNWYVD GVEVHNAKTK PREEQYNSTY RVVSVLTVLH
QDWLNGKEYK CKVSNKALPA PIEKTISKAK GQPREPQVYT
LPPSREEMTK NQVSLTCLVK GFYPSDIAVE WESNGQPENN
YKTTPPVLDS DGSFFLYSKL TVDKSRWQQG NVFSCSVMHE
ALHNHYTQKS LSLSPG

[symbol]

wherein X is a lysine (K) or is absent.

A representative IgG4 sequence for the CH2 and CH3 Domains of a variant Fc Domain having extended half-life comprises the substitutions M252Y/S254T/T256E (SEQ ID NO:20):

APEFLGGPSV FLFPPKPKDT L [symbol] I [symbol] R [symbol] PEVT CVVVDVSQED
PEVQFNWYVD GVEVHNAKTK PREEQFNSTY RVVSVLTVLH
QDWLNGKEYK CKVSNKGLPS SIEKTISKAK GQPREPQVYT
LPPSQEEMTK NQVSLTCLVK GFYPSDIAVE WESNGQPENN
YKTTPPVLDS DGSFFLYSRL TVDKSRWQEG NVFSCSVMHE
ALHNHYTQKS LSLSLG

[symbol]

wherein X is a lysine (K) or is absent.

5. Variable Domains

The Variable Domains of an IgG molecule comprise three “Complementarity-Determining Regions” (“CDRs”), which contain the amino acid residues of the antibody that will be in contact with the epitope, as well as intervening non-CDR segments, referred to as “framework regions” (“FR”), which, in general maintain the structure and determine the positioning of the CDR residues, so as to permit such contacting (although certain framework residues may also contact the epitope). Thus, the VL and VH Domains have the structure n-FR1-CDR1-FR2-CDR2-FR3-CDR3-FR4-c. The amino acid sequences of the CDRs determine whether an antibody will be able to bind to a particular epitope. Interaction of an antibody Light Chain with an antibody Heavy Chain and, in particular, interaction of their VL and VH Domains, forms an Epitope-Binding Domain of the antibody.

Amino acids from the Variable Domains of the mature heavy and Light Chains of immunoglobulins are designated by the position of an amino acid in the chain. Kabat (SEQUENCES OF PROTEINS OF IMMUNOLOGICAL INTEREST, 5^{th} Ed. Public Health Service, NH1, MD (1991)) described numerous amino acid sequences for antibodies, identified an amino acid consensus sequence for each subgroup, and assigned a residue number to each amino acid, and the CDRs and FRs are identified as defined by Kabat (it will be understood that CDR_{H}1 as defined by Chothia, C. & Lesk, A. M. ((1987) “Canonical Structures For The Hypervariable Regions Of Immunoglobulins,” J. Mol. Biol. 196:901-917) begins five residues earlier). Kabat's numbering scheme is extendible to antibodies not included in his compendium by aligning the antibody in question with one of the consensus sequences in Kabat by reference to conserved amino acids. This method for assigning residue numbers has become standard in the field and readily identifies amino acids at equivalent positions in different antibodies, including chimeric or humanized variants. For example, an amino acid at position 50 of a human antibody Light Chain occupies the equivalent position to an amino acid at position 50 of a mouse antibody Light Chain. The positions within the VL and VH Domains at which their CDRs commence and end are thus well defined and can be ascertained by inspection of the sequences of the VL and VH Domains (see, e.g., Martin, C. R. (2010) “Protein Sequence and Structure Analysis of Antibody Variable Domains,” In: ANTIBODY ENGINEERING VOL. 2 (Kontermann, R. and Dubel, S. (eds.), Springer-Verlag Berlin Heidelberg, Chapter 3 (pages 33-51)).

Polypeptides that are (or may serve as) the first, second and third CDR of the Light Chain of an antibody are herein respectively designated as: CDR_{L}1 Domain, CDR_{L}2 Domain, and CDR_{L}3 Domain. Similarly, polypeptides that are (or may serve as) the first, second and third CDR of the Heavy Chain of an antibody are herein respectively designated as: CDR_{H}1 Domain, CDR_{H}2 Domain, and CDR_{H}3 Domain. Thus, the terms CDR_{L}1 Domain, CDR_{L}2 Domain, CDR_{L}3 Domain, CDR_{H}1 Domain, CDR_{H}2 Domain, and CDR_{H}3 Domain are directed to polypeptides that when incorporated into a protein cause that protein to be able to bind to a specific epitope regardless of whether such protein is an antibody having Light and Heavy Chains or is a diabody or a single-chain binding molecule (e.g., an scFv, a BiTe, etc.), or is another type of protein. Accordingly, as used herein, the term “Epitope-Binding Domain” denotes a portion of an Antibody-Based Molecule of the present invention capable of immunospecifically binding to an epitope. An Epitope-Binding Domain may contain any 1, 2, 3, 4, or 5 the CDR Domains of an antibody, or may contain all 6 of the CDR Domains of an antibody and, although capable of immunospecifically binding to such epitope, may exhibit an immunospecificity, affinity or selectivity toward such epitope that differs from that of such antibody. Typically, however, an Epitope-Binding Domain will contain all 6 of the CDR Domains of such antibody.

The Epitope-Binding Domain may comprise either a complete Variable Domain fused onto Constant Domains or only the Complementarity-Determining Regions (CDRs) of such Variable Domain grafted to appropriate framework regions. Epitope-Binding Domains may be wild-type or modified by one or more amino acid substitutions.

### Humanization of Antibody-Based Molecules

The invention particularly encompasses Antibody-Based Molecules that comprise a VL and/or VH Domain of a humanized antibody. The term “humanized” antibody refers to a chimeric molecule, generally prepared using recombinant techniques, having an Epitope-Binding Domain of an immunoglobulin from a non-human species and a remaining immunoglobulin structure of the molecule that is based upon the structure and/or sequence of a human immunoglobulin. The polynucleotide sequence of the Variable Domains of such antibodies may be used for genetic manipulation to generate such derivatives and to improve the affinity, or other characteristics of such antibodies. It is known that the variable domains of both heavy and light chains contain three Complementarity Determining Regions (CDRs) which vary in response to the antigens in question and determine binding capability, flanked by four framework regions (FRs) which are relatively conserved in a given species and which putatively provide a scaffolding for the CDRs. When non-human antibodies are prepared with respect to a particular antigen, the variable domains can be “reshaped” or “humanized.” The general principle in humanizing an antibody involves retaining the basic sequence of the epitope-binding portion of the antibody, while swapping the non-human remainder of the antibody with human antibody sequences. There are four general steps to humanize a monoclonal antibody. These are: (1) determining the nucleotide and predicted amino acid sequence of the starting antibody light and heavy Variable Domains (2) designing the humanized antibody or caninized antibody, i.e., deciding which antibody framework region to use during the humanizing or canonizing process (3) the actual humanizing or caninizing methodologies/techniques and (4) the transfection and expression of the humanized antibody. See, for example, U.S. Pat. Nos. 4,816,567; 5,807,715; 5,866,692; and U.S. Pat. No. 6,331,415; Lobuglio et al. (1989) “Mouse/Human Chimeric Monoclonal Antibody In Man: Kinetics And Immune Response,” Proc. Natl. Acad. Sci. (U.S.A.) 86:4220-4224 (1989). Other references describe rodent CDRs grafted into a human supporting framework region (FR) prior to fusion with an appropriate human antibody Constant Domain (see, for example, Riechmann, L. et al. (1988) “Reshaping Human Antibodies for Therapy,” Nature 332:323-327; and Jones et al. (1986) “Replacing The Complementarity-Determining Regions In A Human Antibody With Those From A Mouse,” Nature 321:522-525. Other methods of humanizing antibodies that may also be utilized are disclosed by Daugherty et al. (1991) “Polymerase Chain Reaction Facilitates The Cloning, CDR-Grafting, And Rapid Expression Of A Murine Monoclonal Antibody Directed Against The CD18 Component Of Leukocyte Integrins,” Nucl. Acids Res. 19:2471-2476 and in U.S. Pat. Nos. 6,180,377; 6,054,297; 5,997,867; and 5,866,692). In some embodiments, humanized antibodies preserve all CDR sequences (for example, a humanized mouse antibody which contains all six CDRs from the mouse antibodies). In other embodiments, humanized antibodies have one or more CDRs (one, two, three, four, five, or six) which differ in sequence relative to the original antibody.

B. Bispecific Molecules

In some embodiments, an Antibody-Based Molecule of the invention is bispecific such as a bispecific antibody or a bispecific diabody. Such bispecific Antibody-Based Molecule may comprise the provided Epitope-Binding Domains of PD-1 and LAG-3 (i.e., a PD-1×LAG-3 bispecific molecule) or the provided Epitope-Binding Domains of PD-L1 and LAG-3 (i.e., a PD-L1×LAG-3 bispecific molecule). The provision of such bispecific Antibody-Based Molecules provides a significant advantage over monospecific antibodies: the capacity to co-ligate PD-1 and LAG-3 on a cell that co-expresses them and/or co-localize a cell that expresses PD-1 and a cell that expresses LAG-3, or the capacity to co-ligate PD-L1 and LAG-3 on a cell that co-expresses them and/or co-localize a cell that expresses PD-L1 and a cell that expresses LAG-3. In certain embodiments, such bispecific Antibody-Based Molecules may bind two different TAs.

### 1. Bispecific Antibodies

A wide variety of recombinant bispecific antibody formats have been developed (see, e.g., PCT Publication Nos. WO 2008/003116, WO 2009/132876, WO 2008/003103, WO 2007/146968, WO 2009/018386, WO 2012/009544, and WO 2013/070565), most of which use linker peptides either to fuse a further epitope-binding fragment (e.g., an scFv, VL, VH, etc.) to, or within the antibody core (IgA, IgD, IgE, IgG or IgM), or to fuse multiple epitope-binding fragments (e.g., two Fab fragments or scFvs). Alternative formats use linker peptides to fuse an epitope-binding fragment (e.g., an scFv, VL, VH, etc.) to a dimerization domain such as the CH2-CH3 Domain or alternative polypeptides (PCT Publication Nos. WO 2005/070966, WO 2006/107786A WO 2006/107617A, and WO 2007/046893). PCT Publication Nos. WO 2013/174873, WO 2011/133886 and WO 2010/136172 disclose a trispecific antibody in which the CL and CH1 Domains are switched from their respective natural positions and the VL and VH Domains have been diversified (PCT Publication Nos. WO 2008/027236; WO 2010/108127) to allow them to bind to more than one antigen. PCT Publication Nos. WO 2013/163427 and WO 2013/119903 disclose modifying the CH2 Domain to contain a fusion protein adduct comprising a binding domain. PCT Publication Nos. WO 2010/028797, WO2010028796 and WO 2010/028795 disclose recombinant antibodies whose Fc Regions have been replaced with additional VL and VH Domains, so as to form trivalent binding molecules. PCT Publication Nos. WO 2003/025018 and WO2003012069 disclose recombinant diabodies whose individual chains contain scFv Domains. PCT Publication Nos. WO 2013/006544 discloses multivalent Fab molecules that are synthesized as a single polypeptide chain and then subjected to proteolysis to yield heterodimeric structures. PCT Publication Nos. WO 2014/022540, WO 2013/003652, WO 2012/162583, WO 2012/156430, WO 2011/086091, WO 2008/024188, WO 2007/024715, WO 2007/075270, WO 1998/002463, WO 1992/022583 and WO 1991/003493 disclose adding additional binding domains or functional groups to an antibody or an antibody portion (e.g., adding a diabody to the antibody's light chain, or adding additional VL and VH Domains to the antibody's light and heavy chains, or adding a heterologous fusion protein or chaining multiple Fab Domains to one another). Covalently bonding diabodies and trivalent molecules comprising diabody-like domains are described in PCT Publication Nos. WO 2015/184207, WO 2015/184203, WO 2012/162068; WO 2012/018687; WO 2010/080538; and WO 2006/113665, and are provided herein. Accordingly, it is specifically contemplated that the PD-1×LAG-3 bispecific molecules of the present invention may have the structure of any of the above-described formats and may be produced any of the above-described methods.

2. Bispecific Diabodies

Diabodies of the present invention are stable, covalently bonded heterodimeric non-mono-specific diabodies, see, e.g., Chichili, G. R. et al. (2015) “A CD3×CD123 Bispecific DART For Redirecting Host T Cells To Myelogenous Leukemia: Preclinical Activity And Safety In Nonhuman Primates,” Sci. Transl. Med. 7(289):289ra82; Veri, M. C. et al. (2010) “Therapeutic Control Of B Cell Activation Via Recruitment Of Fcgamma Receptor IIB (CD32B) Inhibitory Function With A Novel Bispecific Antibody Scaffold,” Arthritis Rheum. 62(7):1933-1943; Moore, P. A. et al. (2011) “Application Of Dual Affinity Retargeting Molecules To Achieve Optimal Redirected T cell Killing Of B-Cell Lymphoma,” Blood 117(17):4542-4551; US Patent Publication Nos. 2007/0004909; 2009/0060910; 2010/0174053; 20130295121; 2014/0099318; 2015/0175697; 2016/0017038; 2016/0194396; 2016/0200827; and 2017/0247452. Such diabodies comprise two or more covalently complexed polypeptide chains and involve engineering one or more cysteine residues into each of the employed polypeptide species. For example, the addition of a cysteine residue to the C-terminus of such constructs has been shown to allow disulfide bonding between the polypeptide chains, stabilizing the resulting heterodimer without interfering with the binding characteristics of the bivalent molecule. Such diabodies also comprise a domain that serves to promote heterodimerization (a “Heterodimer-Promoting Domain”) of the polypeptide chains.

The diabody constructs of the present invention are covalently complexed diabodies composed of polypeptides, and may be composed of two, three, four or more than four polypeptide chains. As used herein, the term “composed of” is intended to be open-ended, such that a diabody of the present invention that is composed of two polypeptide chains may possess additional polypeptide chains. Such chains may have the same sequence as another polypeptide chain of the diabody, or may be different in sequence from any other polypeptide chain of the diabody. Diabodies of the present invention may be designed to comprise Fc Domains.

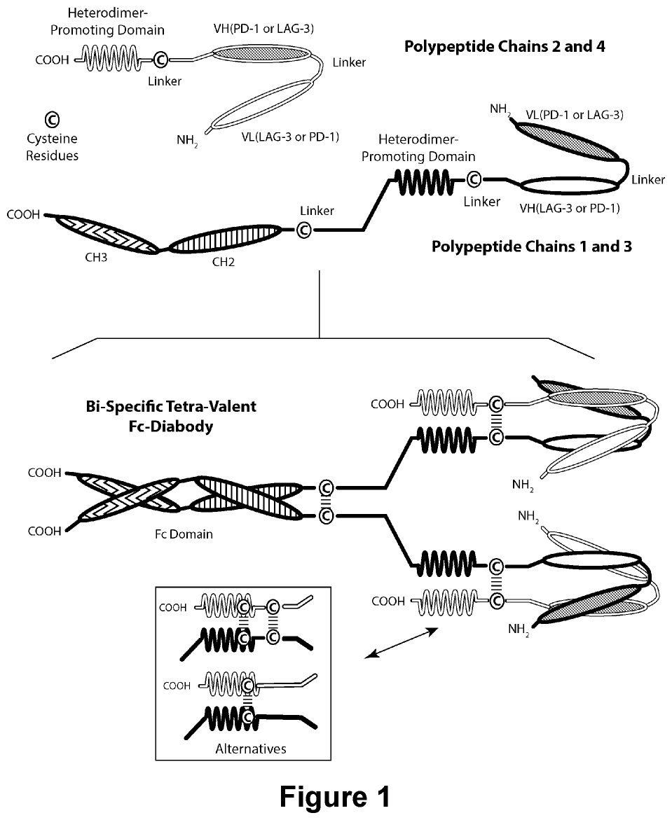

In certain embodiments, the diabodies of the invention, are four chain, Fc Domain-containing diabody having two binding sites specific for a first epitope, two binding sites specific for a second epitope, an Fc Domain, and cysteine-containing E/K-coil Heterodimer-Promoting Domains. The general structure of such diabodies is provided in FIG. 1.

The bispecific diabodies of the present invention are engineered so that such first and second polypeptides covalently bond to one another via cysteine residues along their length. Such cysteine residues may be introduced into an intervening linker (Linker 1; e.g., GGGSGGGG (SEQ ID NO:21)), that separates the VL and VH Domains of the polypeptides. Alternatively, and more preferably, a second peptide that comprises a cysteine residue (Linker 2) is introduced into each polypeptide chain, for example, at a position N-terminal to the VL domain or C-terminal to the VH domain of such polypeptide chain. A preferred sequence for such Linker 2 is SEQ ID NO:22: GGCGGG. Additionally or optionally, cysteine residues may be introduced into other domains, examples of which are provided below.

In certain embodiments, the Heterodimer-Promoting Domains of the present invention will comprise tandemly repeated coil domains of opposing charge. Thus, in one embodiment one of the polypeptide chains will be engineered to contain an “E-coil” domain (SEQ ID NO:23: [symbol] VAAL [symbol] K- [symbol] VAAL [symbol] K- [symbol] VAAL [symbol] K- [symbol] VAAL [symbol] K) whose residues will form a negative charge at pH 7, while the other of the two polypeptide chains will be engineered to contain a “K-coil” domain (SEQ ID NO:24 [symbol] VAA [symbol] E- [symbol] VAAL [symbol] E- [symbol] VAAL [symbol] E- [symbol] VAAL [symbol] E) whose residues will form a positive charge at pH 7. The presence of such charged domains promotes association between the first and second polypeptides, and thus fosters heterodimerization. It is immaterial which coil is provided to the first or second polypeptide chains.

In another embodiment, a Heterodimer-Promoting Domain in which one of the four tandem “E-coil” Helical Domains of SEQ ID NO:23 has been modified to contain a cysteine residue (e.g., [symbol] VAACE K- [symbol] VAAL [symbol] K- [symbol] VAAL [symbol] K- [symbol] VAAL [symbol] K (SEQ ID NO:25) is utilized. Likewise, in another embodiment, a Heterodimer-Promoting Domain in which one of the four tandem “K-coil” Helical Domains of SEQ ID NO:24 has been modified to contain a cysteine residue (e.g., [symbol] VAACE- [symbol] VAAL [symbol] E- [symbol] VAAL [symbol] E- [symbol] VAAL [symbol] E (SEQ ID NO:26) is utilized. Such embodiments are advantageously combined so that the Heterodimer-Promoting Domains of SEQ ID NO:25 and the Heterodimer-Promoting Domains of SEQ ID NO:26 are employed.

Such diabodies are thus engineered so that pairs of their polypeptide chains covalently bond to one another via one or more cysteine residues positioned along their length to produce a covalently associated molecular complex. Such cysteine residues may be introduced into the intervening Linker that separates the VL and VH Domains of the polypeptides. Alternatively, one or more Linkers (e.g., Linker 2, Linker 3, etc.) may contain a cysteine residue. In specific embodiments, one or more coil domains of a coil-containing Heterodimer-Promoting Domain will comprise an amino acid substitution that incorporates a cysteine residue as in SEQ ID NO:25 or SEQ ID NO:26. An alternative, Linker 2 sequence lacking a cysteine residues is SEQ ID NO:27: ASTKG may be employed with cysteine residue containing Heterodimer-Promoting Domains.

The bispecific diabodies of the present invention are preferably engineered such that they possess IgG CH2-CH3 Domains that are capable of complexing together to form an Fc Region. In certain embodiments, the bispecific diabodies of the present invention comprise human IgG CH2-CH3 Domains. Representative human IgG CH2-CH3 Domains are provided above and include CH2-CH3 Domains that have been engineered to modulate effector function and/or serum half-life.

In certain embodiments, the bispecific diabodies of the present invention are engineered with an intervening linker peptide (Linker 3) linking CH2 and CH3 Domains to the Heterodimer-Promoting Domains. Preferably Linker 3 is at a position C-terminal to the Heterodimer-Promoting Domain. Linkers that may be employed in the PD-1×LAG-3 bispecific diabodies of the present invention include: GGGS (SEQ ID NO:28), LGGGSG (SEQ ID NO:29), ASTKG (SEQ ID NO:27), LEPKSS (SEQ ID NO:30), APSSS (SEQ ID NO:31), and APSSSPME (SEQ ID NO:32), GGC, and GGG. Linker 3 may comprise a portion of an IgG hinge region alone or in addition to other linker sequences. Representative hinge regions include: DKTHTCPPCP (SEQ ID NO:33) or EPKSCDKTHTCPPCP (SEQ ID NO:7) from IgG1, ERKCCVECPPCP (SEQ ID NO:8) from IgG2, ESKYGPPCPSCP (SEQ ID NO:10) from IgG4, and ESKYGPPCPPCP (SEQ ID NO:11) an IgG4 hinge variant comprising a stabilizing S228P substitution to reduce strand exchange ((Lu et al., (2008) “The Effect Of A Point Mutation On The Stability Of IgG4 As Monitored By Analytical Ultracentrifugation,” J. Pharmaceutical Sciences 97:960-969) to reduce the incidence of strand exchange). In certain embodiments, Linker 3 may further comprise GGG, for example GGGDKTHTCPPCP (SEQ ID NO:34).

II. Antibody-Based Molecules that Bind to PD-1 (or PD-L1) and/or LAG-3

The present invention specifically contemplates compositions and methods that include or employ:
(1) a PD-1×LAG-3 bispecific molecule;
(2) a monospecific PD-1-Binding Molecule, and a monospecific LAG-3-Binding Molecule;
(3) a PD-L1×LAG-3 bispecific molecule; or
(4) a monospecific PD-L1-Binding Molecule, and a monospecific LAG-3-Binding Molecule;
   wherein such monospecific binding molecule is an intact antibody, and such bispecific molecule is a diabody or a bispecific antibody.

Antibody-Based Molecules that immunospecifically bind to human PD-1 (e.g., monospecific PD-1-Binding Molecules or PD-1×LAG-3 bispecific molecules) that may be used in accordance with the present invention will comprise at least one Epitope-Binding Domain that immunospecifically binds an epitope of PD-1 (a PD-1-Binding Domain).

Antibody-Based Molecules that immunospecifically bind to human PD-L1 (i.e., monospecific PD-L1-Binding Molecules or PD-L1×LAG-3 bispecific molecules) that may be used in accordance with the present invention will comprise at least one Epitope-Binding Domain that immunospecifically binds an epitope of PD-1 (a PD-L1-Binding Domain).

Antibody-Based Molecules that immunospecifically bind to human LAG-3 (i.e., monospecific LAG-3-Binding Molecules, PD-1×LAG-3 (or PD-L1×LAG-3) bispecific molecules) that may be used in accordance with the present invention will comprise at least one Epitope-Binding Domain that immunospecifically binds an epitope of LAG-3 (a LAG-3-Binding Domain).

In certain embodiments, the present invention contemplates Antibody-Based Molecules that comprise a PD-1-Binding Domain, a PD-L1-Binding Domain, and/or a LAG-3-Binding Domain, that further comprise an Fc Domain. In one embodiment, the Fc Domain of such molecules is a wild-type IgG1, IgG2, IgG3, or IgG4 Fc Domain.

The present invention contemplates monospecific Antibody-Based Molecules that comprise a PD-1-Binding Domain, a PD-L1-Binding Domain, or a LAG-3-Binding Domain comprise a variant Fc Domain having little or no ADCC activity. The present invention also contemplates bispecific Antibody-Based Molecules (e.g., diabodies) that comprise Epitope-Binding Domains that are immunospecific for PD-1 and LAG-3, or are immunospecific for PD-L1 and LAG-3, that comprise an Fc Domain having little or no ADCC activity. In one embodiment, such molecules comprise a variant IgG1 Fc Domain comprising a substitution at position 234 with alanine and a substitution at position 235 with alanine (234A, 235A), as numbered by the EU index as in Kabat. In another embodiment, such molecules comprise an IgG4 Fc Domain, and optionally comprise a stabilized IgG4 Hinge Region (see, e.g., SEQ ID NO:11).

In certain embodiments, Antibody-Based Molecules that comprise a PD-1-Binding Domain, a PD-L1-Binding Domain, and/or a LAG-3-Binding Domain comprise a variant Fc Domain comprising one or more mutations which extend serum half-life. In one embodiment, such molecules comprise a variant Fc Domain comprising a substitution at position 252 with tyrosine, 254 with threonine, and 256 with glutamate (252Y, 254T and 256E), as numbered by the EU index as in Kabat.

The present invention also encompasses Antibody-Based Molecules that comprise a PD-1-Binding Domain, a PD-L1-Binding Domain, and/or a LAG-3-Binding Domain which further comprise an Fc Domain wherein such Fc Domain comprises:

(a) one or more mutations which reduced or eliminates ADCC; and/or

(b) one or more mutations which extend serum half-life.

In one embodiment, Antibody-Based Molecules that comprise a PD-1-Binding Domain, a PD-L1-Binding Domain, and/or a LAG-3-Binding Domain comprise a variant IgG1 Fc Domain comprising the substitutions: L234A/L235A/M252Y/S254T/T256E (SEQ ID NO:19), as numbered by the EU index as in Kabat.

In another embodiment, Antibody-Based Molecules that comprise a PD-1-Binding Domain, a PD-L1-Binding Domain, and/or a LAG-3-Binding Domain comprise a variant IgG4 Fc Domain comprising the substitutions: M252Y/S254T/T256E (SEQ ID NO:20), as numbered by the EU index as in Kabat.

A. PD-1-Binding Domains and Molecules

In one embodiment, a PD-1-Binding Domain comprises the CDRs of the VL and VH Domains of SEQ ID NO:35 and SEQ ID NO:39. In another embodiment, a PD-1-Binding Domain comprises the humanized VL and VH Domains of SEQ ID NO:36 and SEQ ID NO:39.

The amino acid sequence of such humanized VL_{PD-}1 Domain is SEQ ID NO:35):

EIVLTQSPAT LSLSPGERAT LSC [symbol] [symbol] WF
QQKPGQPPKL LIH [symbol] GVPSRFSGSG SGTDFTLTIS
SLEPEDFAVY FC [symbol] [symbol] FGGGTKVEI K

The CDRs of such VL_{PD-}1 are:

CDR_{L}1 SEQ ID NO: 36:
RASESVDNYGMSFMN;
CDR_{L}2 SEQ ID NO: 37:
AASNQGS;
and
CDR_{L}3 SEQ ID NO: 38:
QQSKEVPYT.

The amino acid sequence of such humanized VH_{PD-}1 Domain is SEQ ID NO:39:

QVQLVQSGAE VKKPGASVKV SCKASGYSFT [symbol] WVRQA
PGQGLEWIG [symbol] [symbol] [symbol] RVTI TVDKSTSTAY
MELSSLRSED TAVYYCAR [symbol] [symbol] WG QGTLVTVSS

The CDRs of such VH_{PD-}1 Domain are:

CDR_{H}1 SEQ ID NO: 40:
SYWMN;
CDR_{H}2 SEQ ID NO: 41:
VIHPSDSETWLDQKFKD;
and
CDR_{H}3 SEQ ID NO: 42:
EHYGTSPFAY.

Alternative PD-1-Binding Domains, and molecules comprising the same have been described, and include, but are not limited to those presented in Table 1, and which may be referred to here by a common name or, an INN designation.

TABLE 1
PD-1-Binding Domains/Molecules

| Designation | Reference(s) |
|---|---|
| Balstilimab (CAS Reg. No.: 2148321-77-9, also | WHO Drug Information 2019, |
| known as AGEN2034, being developed by | Recommended INN: List 82, 33(3)): |
| Agenus) | 611-612 |
| Budigalimab (CAS Reg. No.: 2098225-93-3, also | WHO Drug Information 2019, |
| known as ABBV-181, PR-1648817, being | Recommended INN: List 81, 33(1): |
| developed by Abbvie) | 56-57 |
| Camrelizumab (CAS Reg. No.: 1798286-48-2, | WHO Drug Information 2017, |
| also known as SHR-1210, and marketed in China | Recommended INN: List 77, 31(1): |
| as AiRuiKa ™ by Shanghai Hengrui | 73-74 |
| Pharmaceuticals) | |
| Cemiplimab (CAS Reg. No.: 1801342-60-8, also | WHO Drug Information 2019, |
| known as REGN-2810, SAR-439684, and | Recommended INN: List 81, 33(1): |
| marketed as LIBTAYO ®, by Sanofi &Regeneron | 57-58 |
| Pharmaceuticals,) | |
| Cetrelimab (CAS Reg. No.: 2050478-92-5, also | WHO Drug Information 2019, |
| known as JNJ-63723283, being developed by | Recommended INN: List 80, 32(3): |
| Janssen Biotech) | 436-437 |
| Dostarlimab (CAS Reg. No.: 2022215-59-2, also | WHO Drug Information 2019, |
| known as ANB-011, TSR-042, being developed | Recommended INN: List 81, 33(1): |
| by Tesero) | 65-66 |
| Ezabenlimab (CAS Reg. No.: 2249882-54-8, | WHO Drug Information 2019, |
| also known as BI754091, being developed by | Proposed INN: List 122, 33(4): |
| Boehringer Ingelheim) | 834-835 |
| Lodapolimab (CAS Reg. No.: 2118349-31-6, also | WHO Drug Information 2019, |
| known as LY3300054, being developed by Eli | Proposed INN: List 121, 33(2): |
| Lilly) | 287-288 |
| Nivolumab (CAS Reg. No.: 946414-94-4, also | WHO Drug Information, 2013, |
| known as 5C4, BMS-936558, ONO-4538, MDX- | Recommended INN: List 69, 27(1): |
| 1106, and marketed as OPDIVO ® by Bristol- | 68-69 |
| Myers Squibb) | |
| Pembrolizumab (formerly known as | WHO Drug Information, 2014, |
| lambrolizumab), CAS Reg. No.: 1374853-91-4, | Recommended INN: List 75, 28(3): |
| also known as MK-3475, SCH-900475, and | 407 |
| marketed as KEYTRUDA ® by Merck) | |
| Prolgolimab (CAS Reg. No.: 2093956-19-3, also | WHO Drug Information 2019, |
| known as BCD-100, being developed by CJSC | Recommended INN: List 81, 33(1): |
| Biocad) | 102-103 |
| Retifanlimab (CAS Reg. No.: 2079108-44-2, also | WHO Drug Information 2019, |
| known as MGA012, INCMGA-00012, being | Recommended INN: List 82, 33(1): |
| developed by Incyte and MacroGenics) | 611-612 |
| Sasanlimab (CAS Reg. No.: 2206792-50-7, also | WHO Drug Information 2019, |
| known as PF-06801591, mAb7, being developed | Proposed INN: List 121, 33(2): |
| by Pfizer) | 330-331 |
| Serplulimab (CAS Reg. No.: 2231029-82-4, also | WHO Drug Information 2019, |
| known as HLX10, being developed by Henlix) | Proposed INN: List 121, 33(2): |
| | 332-333 |
| Sintilimab (CAS Reg. No.: 2072873-06-2, also | WHO Drug Information 2019, |
| known as IBI-308, IBI308, and marketed in | Recommended INN: List 81, 33(1): |
| China as TYVYT ® by Innovent Biologics and | 112-113 |
| Eli Lilly. | |
| Spartalizumab (CAS Reg. No.: 1935694-88-4, | WHO Drug Information 2018, |
| also known as NPVPDR001, NVS240118, | Recommended INN: List 79, 32(1): |
| PDR001, being developed by Novartis) | 161-162 |
| Tislelizumab (CAS Reg. No.: 1858168-59-8, also | WHO Drug Information 2018, |
| known as BGB-A317, being developed by | Recommended INN: List 79, 32(1): |
| Beigene) | 174-175 |
| Toripalimab (CAS Reg. No.: 1924598-82-2, also | WHO Drug Information 2019, |
| known as JS001, being developed by Shanghai | Recommended INN: List 81, 33(1): |
| Junshi Biosciences) | 124-125 |
| PD1-17; PD1-28; PD1-33; PD1-35; and PD1-F2 | U.S. Pat. No. 7,488,802 |
| 17D8; 2D3; 4H1; 5C4; 4A11; 7D3; and 5F4 | U.S. Pat. No. 8,008,449 |
| hPD-1.08A; hPD-1.09A; 109A; K09A; 409A; | U.S. Pat. No. 8,354,509 |
| h409A11; h409A16; h409A17; Codon optimized | |
| 109A; and Codon optimized 409A | |
| 1B8; 20B3.1; 7G3; 3H4; 2.3A9; 1G7; 1.8A10; | U.S. Pat. No. 8,168,757 |
| 28.11; 6D10 | |
| 1E3; 1E8; and 1H3 | US 2014/0044738 |
| 9A2; 10B11; 6E9; APE1922; APE1923; | U.S. Pat. No. 9,815,897 |
| APE1924; APE1950; APE1963; and APE2058 | |
| EH12.2H7 | U.S. Pat. No. 9,102,727 |
| GA1; GA2; GB1; GB6; GH1; A2; C7; H7; SH- | US 2014/0356363 |
| A4; SH-A9; RG1H10; RG1H11; RG2H7; | |
| RG2H10; RG3E12; RG4A6; RG5D9; RG1H10- | |
| H2A-22-1S; RG1H10-H2A-27-2S; RG1H10-3C; | |
| RG1H10-16C; RG1H10-17C; RG1H10-19C; | |
| RG1H10-21C; and RG1H10-23C2 | |
| H1M7789N; H1M7799N; H1M7800N; | US 2015/0203579 |
| H2M7780N; H2M7788N; H2M7790N; | |
| H2M7791N; H2M7794N; H2M7795N; | |
| H2M7796N; H2M7798N; H4H9019P; | |
| H4H7798N; H4xH9034P2; H4xH9035P2; | |
| H4xH9037P2; H4xH9045P2; H4xH9048P2; | |
| H4H9057P2; H4H9068P2; H4xH9119P2; | |
| H4xH9120P2; H4Xh9128p2; H4Xh9135p2; | |
| H4Xh9145p2; H4Xh8992p; H4Xh8999p; and | |
| H4Xh9008p; | |
| mAb1; mAb2; mAb3; mAb4; mAb7; mAb8; | US 2016/0159905 |
| mAb9; mAb10; mAb11; mAb12; mAb13; | |
| mAb14; mAb15; and mAb16 | |
| 246A10; 244C8; 413D2; 393C5; 388D4; 413E1; | US 2016/0319019 |
| 244C8-1; 244C8-2; 244C8-3; 388D4-1; 388D4-2; | |
| and 388D4-3 | |
| Mu317; mu326; 317-4B6; 326-4A3; 317-4B2; | U.S. Pat. No. 8,735,553 |
| 317-4B5; 317-1; 326-3B1; 326-3G1; 326-1; 317- | |
| 3A1; 317-3C1; 317-3E1; 317-3G1; 317-3H1; | |
| 317-3I1; 317-4B1; 317-4B3; 317-4B4; 317-4A2; | |
| 326-3A1; 326-3C1; 326-3D1; 326-3E1; 326-3F1; | |
| 326-3B N55D; 326-4A1; 326-4A2BGB-A317 | |
| 22A5; 6El; lODl, 4C1; 7D3; 13Fl; 14A6; 15H5; | US 2017/267762 |
| 5A8; 7A4; and humanized versions of the same | |
| 1E9; hlE9-l; hlE9-2; hlE9-4; hlE9-5; 4B10; | US 2018/142022 |
| h4B10-1; h4B10-2; h4B10-3; 1B10; 10B4; A09; | |
| C07; F09; G08; G10; H08; H09; and 1353-G10 | |
| M136-M13-MHC723; m136-M14-MHC724; | US 2017/0044259 |
| m136-M19-MHC725; m245-M3-MHC728; | |
| m245-M5-MHC729; A1.0; A1.6; Ba2; Bb2/C1.1; | |
| and D4 | |
| PD-1 mAb 1; PD-1 mAb 2; PD-1 mAb 3; PD-1 | US 2017/019846 |
| mAb 4; PD-1 mAb 5; PD-1 mAb 6; PD-1 mAb 7; | |
| PD-1 mAb 8; PD-1 mAb 9; PD-1 mAb 10; PD-1 | |
| mAb 11; PD-1 mAb 12; PD-1 mAb 13; PD-1 | |
| mAb 14; PD-1 mAb 15; and humanized versions | |
| of the same: hPD-1 mAb 2; hPD-1 mAb 7; hPD-1 | |
| mAb 9; hPD-1 mAb 15; | |
| PD1B11; PD1B70; PD1B71; PD1B114 and | US 20017/079112 |
| affinity-matured variants there of: PD1B149; | |
| PD1B160; PD1B162; PD1B164; PD1B183; | |
| PD1B184; PD1B185; PD1B187; PD1B192; | |
| PD1B175; PD1B177; PD1B194; PD1B195; | |
| PD1B196; PD1B197; PD1B198; PD1B199; | |
| PD1B200; PD1B201 | |
| BAP049-hum01; BAP049-hum02; BAP049- | US 2018/0371093 |
| hum03; BAP049-hum04; BAP049-hum05; | |
| BAP049-hum06; BAP049-hum07; BAP049- | |
| hum08; BAP049-hum09; BAP049-huml0; | |
| BAP049-humll; BAP049-huml2; BAP049- | |
| huml3; BAP049-huml4; BAP049-huml5; | |
| BAP049-huml6; BAP049-Clone-A; BAP049- | |
| Clone-B; BAP049-Clone-C; BAP049-Clone-D; | |
| or BAP049-Clone-E; PDR-001 | |
| AGEN-2034; AGEN-2034w; AGEN2033w; | US 2017/081409 |
| AGEN2046w; AGEN2047w; AGEN2001w; | |
| AGEN2002w; EPl l_pll_B03; EPl l_pll_B05; EPl | |
| l_pll_C02; EPl l_pll_C03 | |
| m136-M13- MHC723; m136-M19- MHC725; | US 2017/044259 |
| m245-M3- MHC728; m245-M5- MHC729; | |
| m136-M14- MHC724; and humanized variants | |
| PD-1 A; PD-1 Ab; PD-1 Ae; PD-1 Af; PD-1 Ba; | |
| PD-1 Bb; PD-1 C; PD-1 Ca; PD-1 D; PD-1 1.0; | |
| PD-1 1.1; PD-1 1.2; PD-1 1.4; PD-1 1.5; PD-1 | |
| 1.6; PD-1 1.7; PD-1 1.9; PD-1 1.10; PD-1 2; PD- | |
| 1 4; CX188 | |
| 244C8; 388D4; 413E1; 246A10; 413D2; and | U.S. Pat. No. 10,239,942 |
| humanized variants D4-HC3 + LC1; D4- | |
| HC1 + LC3; D4-HC3 + LC3; C8-HC1 + LC1; C8- | |
| HC1 + LC3; C8-HC2 + LC1 | |
| PRS-332; VH selected from sequence id nos: 59- | US 2019/010231 |
| 84 and 112-117; and VL selected from sequence | |
| id nos: 85-111 and 118-123 | |
| H005-1 | US 2016/376367 |
| BA08-1 | US 2017/210806 |
| R3A1; R3A2; R4B3; R3B7; R3D6; A2_#1; | US 2018/244779 |
| A2_#2 | |
| BY18.1 | WO 2016/180034 |
| Antibody A, Antibody B, Antibody C, Antibody | US 2017/0044260 |
| D, Antibody E, Antibody F, Antibody G, | |
| Antibody H, Antibody I; 11430 | |
| SHB-128; SHB-152; SHB-168; SHB-617; and | US 2018/346569 |
| humanized variant SSI-361 | |
| E8-3; C2-3; E1-3; F3-3; H8-3; C10-2; G2-1; G3- | U.S. Pat. No. 9,982,052 |
| 2; H2-1; H4-2; C8-1; G10-2; 135C12; 136B4; | |
| 139D6; 136E10; 122F10; 139D6; 137F2 | |
| AB12M3; AB12M4; AB12M5; AB12M6; | US 2018/113258 |
| AB12M7; AB12M8; AB12M9 | |
| 1.7.3 hAb; 1.49.9 hAb; 1.103.11 hAb; 1.139.15 | US 2017/024515; US 2017/025051 |
| hAb; 1.153.7 hAb | |
| 949 and humanized variants including 949 VK1 | U.S. Pat. No. 9,102,728 |
| gL9 gH8b | |
| 948 and humanized variants | U.S. Pat. No. 8,993,731 |
| STM-432 | US 2019/077866 |

It is specifically contemplated that the PD-1-Binding Molecules presented herein may be used directly in the methods of the present invention, or the sequences or polypeptide chains may be employed in the construction of alternative PD-1-Binding Molecules, or PD-1×LAG-3 bispecific molecules.

B. PD-L1-Binding Domains and Molecules

In one embodiment a PD-L1-Binding Domain comprises the CDRs of the VL and VH Domains of SEQ ID NO:43 and SEQ ID NO:47. In another embodiment, a PD-L1-Binding Domain comprises the humanized VL and VH Domains of SEQ ID NO:43 and SEQ ID NO:47.

The amino acid sequence of such humanized VL_{PD-L1} Domain is (SEQ II) NO:43):

DIQMTQSPSS LSASVGDRVT ITCKASQDVN TAVAWYQQKP
GKAPKLLIYW ASTRHTGVPS RFSGSGSGTD FTLTISSLQP
EDFATYYCQQ HYNTPLTFGQ GTKVEIK

The CDRs of such VL_{PD-L}1 are:

CDR_{L}1 SEQ ID NO: 44:
KASQDVNTAVA;
CDR_{L}2 SEQ ID NO: 45:
WASTRHT;
and
CDR_{L}3 SEQ ID NO: 46:
QQHYNTPLT.

The amino acid sequence of such VH_{PD-L1} humanized Domain is (SEQ ID NO:47):

EVQLVESGGG LVQPGGSLRL SCAASGFTFS STYMSWVRQA
PGKGLEWVAY ISIGGGTTYY PDTVKGRFTI SRDNAKNTLY
LQMNSLKTED TAVYYCARQG LPYYFDYWGQ GTLVTVSS

The CDRs of such VH_{PD-L1} are:

CDR_{H}1 SEQ ID NO: 48:
SYTM;
CDR_{H}2 SEQ ID NO: 49:
YISIGGGTTYYPDTVK;
and
CDR_{H}3 SEQ ID NO: 50:
QGLPYYFDY.

Alternative PD-L1-Binding Domains, and molecules comprising the same have been described, and include, but are not limited to those presented in Table 2, and which may be referred to here by a common name or, an INN designation.

TABLE 2
PD-L1-Binding Molecules

| Designation | Reference(s) |
|---|---|
| Adebrelimab (CAS Reg No.: 2247114-85-6, also | WHO Drug Information 2019, |
| known as HTI-1088, SHR-1316, being developed | Proposed INN: List 122, 33(4): |
| by Hengrui Therapeutics) | 804-805 |
| Atezolizumab (CAS Reg. No.: 2118349-31-6, | WHO Drug Information, 2015, |
| also known as MPDL3280A, RG7446, and | Recommended INN: List 74, 29(3): |
| marketed as TECENTRIQ ® by Genentech, Inc.) | 387 |
| Avelumab (CAS Reg. No.: 2118349-31-6, also | WHO Drug Information, 2016, |
| known as MSB-0010718C, MSB0010682, | Recommended INN: List 74, 30(1): |
| MSB0010718C, and marketed as BAVENCIO ® | 100-101 |
| by EMD Serono) | |
| Bintrafusp alfa (CAS Reg. No.: 1918149-01-5, a | WHO Drug Information, 2019, |
| bifunctional fusion protein having 2 extracellular | Recommended INN: List 81, 33(1): |
| domain of TGF-βII (a TGF-β “trap”) fused to a | 52-54 |
| human IgG1 monoclonal antibody against PD-L1, | |
| also known as M7824, being developed by | |
| Merck and GSK) | |
| Cosibelimab (CAS Reg No.: 2216751-26-5, also | WHO Drug Information 2019, |
| known as CK-301, being developed by | Proposed INN: List 121, 33(2): |
| Checkpoint Therapeutics) | 258-259 |
| Durvalumab (CAS Reg. No.: 2118349-31-6, also | WHO Drug Information, 2015, |
| known as MEDI4736, and marketed as | Recommended INN: List 74, 29(3): |
| IMFINZI ® by Astrazeneca) | 393-394 |
| Envafolimab (CAS Reg. No.: 2102192-68-5, a | WHO Drug Information, 2019, |
| single-domain antibody also known as KN-035, | Recommended INN: List 82, 33(3): |
| being developed by Alphamab Co.) | 634-635 |
| Manelimab (CAS Reg No.: 2168561-26-8, also | WHO Drug Information 2019, |
| known as BCD-135, being developed by CJSC | Proposed INN: List 121, 33(2): |
| Biocad) | 289-290 |
| Opucolimab (CAS Reg. No.: 2251771-79-4, also | WHO Drug Information 2019, |
| known as HLX20, being developed by Henlix | Proposed INN: List 122, 33(4): |
| Biotech) | 866-867 |
| Pacmilimab (CAS Reg No.: 2145091-51-4, a | WHO Drug Information 2019, |
| PROBODY ™ also known as CX-072, being | Proposed INN: List 121, 33(2): |
| developed by CytomX Therapeutics) | 312-313 |
| Sugemalimab (CAS Reg. No.: 2256084-03-2, | WHO Drug Information 2019, |
| also known as CS-1001, WBP-315, WBP 3155, | Proposed INN: List 122, 33(4): |
| being developed by CStone Pharmaceuticals) | 892-893 |
| A09-188-1, and affinity-matured and optimized | U.S. Pat. No. 9,624,298 |
| variants: A09-204-1, A09-211-1, A09-212-1, | |
| A09-213-1, A09-214-1, A09-215-1, A09-216-1, | |
| A09-219-1, A09-220-1, A09-221-1, A09-222-1, | |
| A09-223-1, A09-202-1, A09-248-2, A09-239-2, | |
| A09-240-2, A09-241-2, A09-242-2, A09-243-2, | |
| A09-244-2, A09-245-2, A09-246-2, A09-247-2 | |
| YW243.55.S70; 243.55.H1; 243.55.H12; | U.S. Pat. No. 8,217,149 |
| 243.55.H37; 243.55.H70; 243.55.H89; | |
| 243.55.S1; 243.55.5; 243.55.8; 243.55.30; | |
| 243.55.34; 243.55.S37; 243.55.49; 243.55.51; | |
| 243.55.62; 243.55.84 | |
| 2.9D10, 2.7A4, 2.14H9, 3.15G8, 2.20A8, | U.S. Pat. No. 8,779,108B2 |
| 3.18G1, 2.7A4OPT, or 2.14H9OPT | |
| 1B9.2E11.2, 4H1.G10.15, 1A8, 1E4, 8G2, 1D11, | US 2015/0197571 |
| 3A2, 3B11, 3F4, 3H6, 4C1, 4E1, 5A6, 9C12, | |
| 1B4, 1B11, 1F6, 1H8, 1H12, 2D5, 2H11, 3D12, | |
| 4C8, 4C9, 5E10, 5H4, 5H5, 8A1, 9G9, 10A7, and | |
| 10H6 | |
| 1D05, 84G09, 411B08, 411C04, 411D07, | U.S. Pat. No. 9,617,338 |
| 386H03, 386A03, 385F01, 413D08, 413G05, | |
| 413F09, 414B06 | |
| 3G10, 12A4, 10A5, 5F8, 10H10, 1B12, 7H1, 11E6, | U.S. Pat. No. 9,273,135 |
| 12B7, and 13G4 | |
| A1, C2, C4, H12, and H12-GL | US 2017/0319690 |
| Ab- 14, Ab-16, Ab-22, Ab-30, Ab-31, Ab-32, | U.S. Pat. No. 9,828,434 |
| Ab-38, Ab-42, Ab-46, Ab-50, Ab-52, Ab-55, Ab- | |
| 56, and Ab-65 | |
| R2κA3, R2κA4, R2κA6, R2κF4, R2κH5, R2κH6, | US 2016/340429 |
| R2vH3, sR3κA8, sR3κA9, sR3κB2, sR3κB5, | |
| tccR3KA8, tccR3KAl1, tccR3KB7, tccR3KD9, | |
| tccKF10, tctR3KA4, tctR3KF8, R2λA7, R2λB12, | |
| R2λ12, sR3λD7, sR3λE1, tccAF8, tccAD7, | |
| tctR3λH4, KD-033, and others | |
| H2M8306N, H2M8307N, H2M8309N, | U.S. Pat. No. 9,938,345 |
| H2M8310N, H2M8312N, H2M8314N, | |
| H2M8316N, H2M8317N, H2M8321N, | |
| H2M8323N, H2M8718N, H2M8718N2, and | |
| H2M8719N, H1H9323P, H1 H9327P, H1 | |
| H9329P, H1H9336P, H1H9344P2, 1H9345P2, | |
| H1H9351P2, H1H9354P2, H1 H9364P2, | |
| H1H9373P2, H1H9382P2, H1H9387P2, and | |
| H1H9396P2 | |
| clone 8, clone 12, clone 16, clone 18, clone 60; | US 2016/0311903 |
| and optimized variants thereof including: cl; dl; | |
| g7; h9; b10; E10; A05; C05; C10; D08; G09; | |
| G10; G12; Ell; D01; H06; C5H9; C5B10; C5E10; | |
| G12H9; G12B10; G12E10; | |
| BAP058 and humanized variants thereof | U.S. Pat. No. 9,988,452 |
| including: BAP058-hum01, BAP058-hum02, | |
| BAP058-hum03, BAP058-hum04, BAP058- | |
| hum05, BAP058-hum06, BAP058-hum07, | |
| BAP058-hum08, BAP058-hum09, BAP058- | |
| hum10, BAP058-hum11, BAP058-hum12, | |
| BAP058-hum13, BAP058-hum14, BAP058- | |
| hum15, BAP058-hum16, and BAP058-hum17; | |
| FAZ-053 | |
| Mu333, Mu277, and humanized variants thereof | US 2018/215825 |
| including: hu333-2B, hu333-3A2, hu333-3C2 and | |
| hu333-3H2 | |
| 332M1 and humanized variants there of | US 2018/346571 |
| including: 332M7, 332M72, and 332M8 | |
| PDL1.1; PDL1.2 | U.S. Pat. No. 8,741,295 |
| 13C5, 5G9, 5G11, 8C6, 7B4, 4D1, 4A8, 8H4, | US 2017/0204184 |
| 8H3, 15F1; and humanized variants thereof | |
| including hu5G11; hu13C5; | |
| PDL1-56 dAb; Hu56V1; Hu56V2; Hu56V3; | US 2018/0291103 |
| Hu56V4; Hu56V5; and KN035 | |
| 1.4.1, 1.14.4, 1.20.15 and 1.46.11 | WO 2017/020858 |
| CTI-07, CTI-09, CTI-48, CTI-49, CTI-50, CTI- | US 2018/0002424 |
| 76, CTI-77, CTI-78, CTI-57, or CTI-58 | |
| 92; 24D5; 29H1; 9_2-1; 9_2-2; 9_2-3; 9_2-4; | US 2018/0334504 |
| 9_2-5; 9_2-6; 9_2-7; 9_2-8; 9_2-9; 9_2-10; | |
| 24D5-H; HRP00049; HRP-00052 | |
| 5F10; 9F6; 5C10 and humanized variants thereof | US 2018/0305464 |
| including 5C10H1L1; 5C10H1L2; 5C10H2L1; | |
| and 5C10H2L2 | |
| 4B6, 26F5, 21F11, 23A11, 23F11 and 22C9; | WO 2017/161976 |
| BM-GT, BM-ME, 4B6-H3L4, 4B6- | |
| H4L3, 23F11-H4L4, 23F11-H4L6, 23F11- | |
| H6L4, 23F11-H6L6, 23A11-H3L3, 23A11- | |
| H3L5, 23A11-H5L3 and 23A11-H5L5; | |
| 3C5-2G12 and humanized variants thereof | WO 2017/196867 |
| including h3C5H1-h3C5L1; h3C5H2- h3C5L2; | |
| h3C5H3- h3C5L2; h3C5H4-h3C5L2; | |
| 29E.2A3 and 24F.10C12 | U.S. Pat. No. 8,552,154 |
| PD-L1 MAB-1, PD-L1 MAB-2, PD-L1 MAB-3, | WO 2020/041404 |
| and humanized variants there of including hPD- | |
| L1 MAB2, hPD-L1-MAB-3 | |

It is specifically contemplated that the PD-L1-Binding Molecules presented herein may be used directly in the methods of the present invention, or the sequences or polypeptide chains may be employed in the construction of alternative PD-L1-Binding Molecules, or PD-L1×LAG-3 bispecific molecules.

C. LAG-3-Binding Domains and Molecules

In one embodiment, a LAG-3-Binding Domain comprises the CDRs of the VL and VH Domains of SEQ II) NO:51 and SEQ II) NO:55. In another embodiment, a LAG-3-Binding Domain comprises the humanized VL and VH Domains of SEQ ID NO:51 and SEQ ID NO:55.

The amino acid sequence of such humanized VL_{LAG-3} Domain is (SEQ ID NO:51):

DIQMTQSPSS LSASVGDRVT ITCRASQDVS SVVAWYQQKP
GKAPKLLIYS ASYRYTGVPS RFSGSGSGTD FTLTISSLQP
EDFATYYCQQ HYSTPWTFGG GTKLEIK

The CDRs of such VL_{LAG-3} Domain comprises:

CDR_{L}1 seq ID NO: 52:
RASQDVSSVVA;
CDR_{L}2 SEQ ID NO: 53:
SASYRYT;
and
CDR_{L}3 SEQ ID NO: 54:
QQHYSTPWT.

The amino acid sequence of such humanized VH_{LAG-3} Domain is (SEQ ID NO:55):

QVQLVQSGAE VKKPGASVKV SCKASGYTFT DYNMDWVRQA
PGQGLEWMGD INPDNGVTIY NQKFEGRVTM TTDTSTSTAY
MELRSLRSDD TAVYYCAREA DYFYFDYWGQ GTTLTVSS

The CDRs of such VH_{LAG-3} Domain comprises:

CDR_{H}1 SEQ ID NO: 56:
DYNMD;
CDR_{H}2 SEQ ID NO: 57:
DINPDNGVTIYNQKFEG;
and
CDR_{H}3 SEQ ID NO: 58:
EADYFYFDY.

Alternative LAG-3-Binding Domains, and molecules comprising the same have been described, and include, but are not limited to those presented in Table 3, and which may be referred to here by a common name or, an INN designation.

TABLE 3
LAG-3-Binding Molecules

| Designation | Reference(s) |
|---|---|
| relatlimab (CAS Reg No.: 1673516-98-7, also | WHO Drug Information, 2019, |
| known as BMS-986016, ONO-4482, being | Recommended INN: List 81, 33(1): |
| developed by Bristol-Myers Squibb) | 104-105 |
| ieramilimab (CAS Reg No.: 2137049-37-5, also | WHO Drug Information 2018, |
| known as LAG-525, IMP-701, being developed | Proposed INN: List 120, 32(4): |
| by Novartis | 601-602 |
| encelimab (CAS Reg No.: 2173096-82-5, also | WHO Drug Information 2019, |
| known as TSR-033, being developed by | Proposed INN: List 121, 33(2): |
| Anaptysbio/Tessero) | 265-266 |
| fianlimab (CAS Reg No.: 2126132-98-5, also | WHO Drug Information 2019, |
| known as REGN 3767, being developed by | Proposed INN: List 121, 33(2): |
| Regneron) | 271-272 |
| mavezelimab (CAS Reg No.: 2231068-83-8, also | WHO Drug Information 2019, |
| known as MK-4280, being developed by Merck | Proposed INN: List 121, 33(2): |
| | 290-291 |

It is specifically contemplated that the LAG-3-Binding Molecules presented herein may be used directly in the methods of the present invention, or the sequences or polypeptide chains may be employed in the construction of alternative LAG-3-Binding Molecules, or PD-1×LAG-3 (or PD-L1×LAG-3) bispecific molecules.

D. PD-1×LAG-3 (or PD-L1×LAG-3) Bispecific Molecules

Antibody-Based Molecules that immunospecifically bind both to human PD-1 (or PD-L1) and to human LAG-3 (i.e., PD-1×LAG-3 bispecific molecules, or PD-L1×LAG-3 bispecific molecules) that may be used in accordance with the present invention will comprise at least one Epitope-Binding Domain that immunospecifically binds an epitope of PD-1 (or PD-L1) and at least one Epitope-Binding Domain that immunospecifically binds an epitope of LAG-3.

In certain embodiments, the PD-1×LAG-3 bispecific molecules of the present invention comprise:
(I) a PD-1-Binding Domain comprising a VL Domain (VL_{PD-1}) comprising PD-1-specific CDR_{L}1, CDR_{L}2, and CDR_{L}3, Domains, and a VH Domain (VH_{PD-1}) comprising PD-1-specific CDR_{H}1, CDR_{H}2 and CDR_{H}3 Domains; and
(II) a LAG-3-Binding Domain comprising a VL Domain (VL_{LAG-3}) comprising LAG-3-specific CDR_{L}1, CDR_{L}2, and CDR_{L}3, Domains, and a VH Domain (VH_{LAG-3}) comprising LAG-3-specific CDR_{H}1, CDR_{H}2, and CDR_{H}3, Domains,
wherein the PD-1-Binding Domain and the LAG-3-Binding Domains are selected from those provided in Tables 1 and 3.

In other embodiments, the PD-L1×LAG-3 bispecific molecules of the present invention comprise:
(I) a PD-L1-Binding Domain comprising a VL Domain (VL_{PD-L1}) comprising PD-L1-specific CDR_{L}1, CDR_{L}2, and CDR_{L}3, Domains, and a VH Domain (VH_{PD-L1}) comprising PD-L1-specific CDR_{H}1, CDR_{H}2 and CDR_{H}3 Domains; and
(II) a LAG-3-Binding Domain comprising a VL Domain (VL_{LAG-3}) comprising LAG-3-specific CDR_{L}1, CDR_{L}2, and CDR_{L}3, Domains, and a VH Domain (VH_{LAG-3}) comprising LAG-3-specific CDR_{H}1, CDR_{H}2, and CDR_{H}3, Domains
wherein the PD-L1-Binding Domain and the LAG-3-Binding Domains are selected from those provided in Tables 2 and 3.

One embodiment of the present invention relates to PD-1×LAG-3 (or PD-L1×LAG-3) bispecific molecules that comprise an Fc Domain. In one embodiment, PD-1×LAG-3 (or PD-L1×LAG-3) bispecific molecules comprise an Fc Domain having little or no ADCC activity. In one embodiment, PD-1×LAG-3 (or PD-L1×LAG-3) bispecific molecules comprise an Fc Domain having little or no ADCC activity and comprising one or more mutations which extend serum half-life.

In certain embodiments, the PD-1×LAG-3 bispecific molecules of the present invention are PD-1×LAG-3 bispecific diabodies, preferably four chain, Fc Domain-containing diabody having two binding sites specific for PD-1, two binding sites specific for LAG-3, an Fc Domain, and cysteine-containing E/K-coil Heterodimer-Promoting Domains. The general structure of representative PD-1×LAG-3 bispecific diabodies is provided in FIG. 1. Such molecules comprise a VL and VH Domain of an antibody that binds to PD-1 (VL_{PD-}1 and VH_{PD-1}, respectively) and also a VL and VH Domain of an antibody that binds to LAG-3 (VL_{LAG-3} and VH_{LAG-3}, respectively). Thus, such PD-1×LAG-3 bispecific diabodies are capable of specifically binding to an epitope of PD-1 and to an epitope of LAG-3.

1. DART-I

“DART-I” (also known as “MGD013” and tebotelimab) is a representative PD-1×LAG-3 bispecific molecule of the invention. DART-I is a bispecific, four chain, Fc Domain-containing diabody having two binding sites specific for PD-1, two binding sites specific for LAG-3, a variant IgG4 Fc Domain engineered for extended half-life, and cysteine-containing E/K-coil Heterodimer-Promoting Domains. DART-I comprises four polypeptide chains having the amino acid sequences summarized in Table 4. The amino acid sequences are described in further detail below.

TABLE 4
DART-I SEQ ID NOs

| | Substituent Polypeptides (in the |
|---|---|
| DART-I (tebotelimab) | N-Terminal to C-Terminal Direction) |
| First and Third | SEQ ID NO: 51 |
| Polypeptide Chains | SEQ ID NO: 21 |
| (SEQ ID NO: 59) | SEQ ID NO: 39 |
| | SEQ ID NO: 22 |
| | SEQ ID NO: 25 |
| | SEQ ID NO: 11 |
| | SEQ ID NO: 20 |
| Second and Fourth | SEQ ID NO: 35 |
| Polypeptide Chains | SEQ ID NO: 21 |
| (SEQ ID NO: 60) | SEQ ID NO: 55 |
| | SEQ ID NO: 22 |
| | SEQ ID NO: 26 |

The first and third polypeptide chains of DART-I comprise, in the N-terminal to C-terminal direction: an N-terminus, a VL Domain of a monoclonal antibody capable of binding to LAG-3 (VL_{LAC-3} SEQ ID NO:51); an intervening linker peptide (Linker 1: GGGSGGGG (SEQ ID NO:21)); a VH Domain of a monoclonal antibody capable of binding to PD-1 (VH_{PD-1}) (SEQ ID NO:39); a cysteine-containing intervening linker peptide (Linker 2: GGCGGG (SEQ ID NO:22)); a cysteine-containing Heterodimer-Promoting (E-coil) Domain (EVAACEK-EVAALEK-EVAALEK-EVAALEK (SEQ ID NO:25)); an intervening linker peptide (Linker 3) comprising a stabilized IgG4 hinge region (SEQ ID NO: 11); a variant IgG4 CH2-CH3 Domain comprising substitutions M252Y/S254T/T256E and lacking the C-terminal residue (SEQ ID NO:20); and a C-terminus.

The amino acid sequence of the first and third polypeptide chains of DART-I is (SEQ ID NO:59):

DIQMTQSPSS LSASVGDRVT ITCRASQDVS SVVAWYQQKP
GKAPKLLIYS ASYRYTGVPS RFSGSGSGTD FTLTISSLQP
EDFATYYCQQ HYSTPWTFGG GTKLEIKGGG SGGGGQVQLV
QSGAEVKKPG ASVKVSCKAS GYSFTSYWMN WVRQAPGQGL
EWIGVIHPSD SETWLDQKFK DRVTITVDKS TSTAYMELSS
LRSEDTAVYY CAREHYGTSP FAYWGQGTLV TVSSGGCGGG
EVAACEKEVA ALEKEVAALE KEVAALEKES KYGPPCPPCP
APEFLGGPSV FLFPPKPKDT LYITREPEVT CVVVDVSQED
PEVQFNWYVD GVEVHNAKTK PREEQFNSTY RVVSVLTVLH
QDWLNGKEYK CKVSNKGLPS SIEKTISKAK GQPREPQVYT
LPPSQEEMTK NQVSLTCLVK GFYPSDIAVE WESNGQPENN
YKTTPPVLDS DGSFFLYSRL TVDKSRWQEG NVFSCSVMHE
ALHNHYTQKS LSLSLG

The second and fourth polypeptide chains of DART-I comprise, in the N-terminal to C-terminal direction: an N-terminus, a VL Domain of a monoclonal antibody capable of binding to PD-1 (VL_{PD-1}) (SEQ ID NO:35); an intervening linker peptide (Linker 1: GGGSGGGG (SEQ ID NO:21)); a VH Domain of a monoclonal antibody capable of binding LAG-3 (VH_{LAG-3}) (SEQ ID NO:55); a cysteine-containing intervening linker peptide (Linker 2: GGCGGG (SEQ ID NO:22)); a cysteine-containing Heterodimer-Promoting (K-coil) Domain (KVAACKE-KVAALKE-KVAALKE-KVAALKE (SEQ ID NO:26); and a C-terminus.

The amino acid sequence of the second and fourth polypeptide chains of DART-I is (SEQ ID NO:60):

EIVLTQSPAT LSLSPGERAT LSCRASESVD NYGMSEMNWE
QQKPGQPPKL LIHAASNQGS GVPSRFSGSG SGTDFTLTIS
SLEPEDFAVY FCQQSKEVPY TFGGGTKVEI KGGGSGGGGQ
VQLVQSGAEV KKPGASVKVS CKASGYTFTD YNMDWVRQAP
GQGLEWMGDI NPDNGVTIYN QKFEGRVTMT TDTSTSTAYM
ELRSLRSDDT AVYYCAREAD YFYFDYWGQG TTLTVSSGGC
GGGKVAACKE KVAALKEKVA ALKEKVAALK E

Variants of DART-I may be readily generated by incorporating alternative VH/VL Domains, intervening linkers, Fc Domains, and/or by introducing one or more amino acid substitutions, additions, or deletions. For example, a variant IgG1 Fc Domain engineered to reduce/abolish FcγR bindings and/or ADCC activity and for extended half-life is readily generated by incorporating CH2 and CH3 Domains comprising the substitutions L234A/L235A/M252Y/S254T/T256E (SEQ ID NO:19) instead of SEQ ID NO:20. Linker 3 of such variant may comprise an IgG1 hinge (SEQ ID NO:33, SEQ ID NO:35, or SEQ ID NO:34). Additional linkers and PD-1×LAG-3 bispecific diabodies which may be used in the methods of the present invention are disclosed in WO 2015/200119; and in WO 2017/019846 (see in particular “DART-A,” DART-B,” “DART-C,” “DART-D,” “DART-E,” “DART-F,” and “DART-G”, the sequences of which are described therein in Table 14).

2. Additional PD-1×LAG-3 (or PD-L1×LAG-3) Bispeciic Molecules

Other PD-1×LAG3 bispecific molecules which may be used in the method of the present invention have been described, and include, but are not limited to those presented in Table 5 and father described below.

TABLE 5
PD-1 × LAG-3 (or PD-L1 × LAG-3) Bispecific Molecules

| Designation | Reference(s) |
|---|---|
| PD-1 × LAG3 bispecific molecules comprising the | WO 2017/025498 |
| combination of reported sequence id nos: 5 and 4; 6 and 4; | |
| 3 and 4; 3 and 7; 3 and 8; 9 and 4; 10 and 4; 3 and 11; 3 | |
| and 12 | |
| PD-L1 × LAG3 bispecific molecules designated: FS18-7- | WO 2017/220569 |
| 9/84G09; FS18-7-32/84G09; FS18-7-33/84G09; FS18-7-36/84G09; | |
| FS18-7-58/84G09; FS18-7-62/84G09; FS18-7-65/84G09; | |
| FS18-7-78/84G09; FS18-7-88/84G09; FS18-7-95/84G09 | |
| PD-1 × LAG3 bispecific molecule designated | WO 2018/083087 |
| 57E02 × 51A09-188001 mAbdAb; and numerous PD-1 and | |
| LAG-3 epitope binding domains | |
| PD-1 × LAG3 bispecific molecules comprising the | WO 2018/134279 |
| combination of reported sequence id nos: 74 and 66; 61 | |
| and 75; 85 and 66; 61 and 86; 78 and 62; 65 and 79; 65 | |
| and 81; 78 and 79; 65 and 62; 61 and 66; 76 and 66; 61 | |
| and 77; 80 and 62; 65 and 81 | |
| PD-1 × LAG3 bispecific molecules designated: 0799, | WO 2018/185043 |
| 0927, 0222, 0224, 8970, 8984, 9010, 8310, 8311, 1252, | |
| 8312, 8313, 1088, 0918, 0725 | |
| PD-1 × LAG3 bispecific molecules designated: A, B, C, | WO 2018/217940 |
| E, F, G, H, I, J, K, L, M, N, O, P, Q, R, S, T, U, | |
| PD-1 × LAG3 bispecific molecules designated: 18ASS, | WO 2019/148412 |
| 90ASU, 33ARK | |

PD-1×LAG3 bispecific antibody-lipocalin mutein fusion proteins are described in WO 2017/025498 and WO 2018/134279. Examples of such antibody-lipocalin mutein fusion proteins include anti-PD-1 antibodies having a lipocalin mutein engineered to bind LAG-3 genetically fused to the C-terminus of the heavy chain.

PD-1×LAG-3 bispecific antibody-domain antibody (antibody-dAb) fusion proteins are described in WO 2018/083087. Examples of such antibody-dAb fusion proteins include an anti-LAG-3 antibody having an anti-PD-1 dAb genetically fused to the C-terminus of the heavy chain. PD-1×LAG-3 bispecific antibodies comprising CH/Ck domain exchange (alone on in combination with VH/VL exchange) and/or charged amino acid substitutions in the CH1/CL interfaces are described in WO 2018/185043. Examples of such bispecific antibodies include four polypeptide chain antibodies having one PD-1-Binding Domain and one LAG-3-Binding Domain (1+1 antibody) comprising a crossFab (with VH/VL domain exchange), and three polypeptide chain antibodies having three different polypeptide chains two PD-1-Binding Domains and two LAG-3-Binding Domains (2+2 antibody) comprising two Fab domains having mutations in CH1/CK and two crossFab domains fused at the C-terminus of each heavy chain.

PD-1×LAG-3 bispecific antibodies having a three polypeptide chain Fab×scFvFc structure or a two polypeptide chain scFvFc×cFvFc structure are described in WO 2018/217944 and WO 2018/217940. Examples of such bispecific antibodies comprise an anti-PD1 scFvFc paired with an anti-LAG3 scFvFc hole, and an anti PD1 scFvFc paired with an anti-LAG3 half IgG (heavy chain+light chain).

It is specifically contemplated that the PD-1×LAG-3 bispecific molecules and PD-L1×LAG-3 bispecific molecules presented herein may be used directly in the methods of the present invention. Alternative PD-1×LAG-3 bispecific molecules and PD-L1×LAG-3 bispecific molecules may be generated that comprise the 6 CDRs (or VL and VH Domains) of any of the PD-1, PD-L1, and LAG-3-Binding Molecules provided herein (see, e.g., SEQ ID NOs:35-58, and Tables 1-5).

III. Antibody-Based Molecules That Bind to a TA

Antibody-Based Molecules that immunospecifically bind to a Tumor Antigen (TA) (i.e., TA-Binding Molecules) that may be used in accordance with the present invention will comprise at least one Epitope-Binding Domain that immunospecifically binds an epitope of such TA (a TA-Binding Domain).

In certain embodiments, the present invention contemplates Antibody-Based Molecules that comprise a TA-Binding Domain that further comprise an Fc Domain. In one embodiment, the Fc Domain of TA-Binding molecules is a wild-type IgG1, IgG2, IgG3, or IgG4 Fc Domain. In another embodiment, the Fc Domain of TA molecules is an ADCC-Enhanced Fc Domain.

The present invention also encompasses TA-Binding Molecules which comprise an Fc Domain wherein such Fc Domain comprises:

(a) one or more mutations and/or modification which enhances ADCC; and/or

(b) one or more mutations which extend serum half-life.

In one embodiment, TA-Binding Molecules comprise the FcMT1 ADCC-Enhanced Fc Domain (SEQ ID NO:16), the FcMT2 ADCC-Enhanced Fc Domain (SEQ ID NO:17), or the FcMT3 ADCC-Enhanced Fc Domain (SEQ ID NO:18).

A. Tumor Antigens

The present invention specifically contemplates compositions and methods that include or employ a TA-Binding Molecule and:
(1) a PD-1×LAG-3 bispecific molecule;
(2) a monospecific PD-1-Binding Molecule, and a monospecific LAG-3-Binding Molecule;
(3) a PD-L1×LAG-3 bispecific molecule; or
(4) a monospecific PD-L1-Binding Molecule, and a monospecific LAG-3-Binding Molecule,
   wherein such monospecific binding molecule is an intact antibody, and such bispecific molecule is a diabody or a bispecific antibody. In certain embodiments the TA-Binding Molecule comprises an ADCC-Enhanced Fc Domain.

Tumor Antigens that may be bound by such TA-Binding Molecules include, but are not limited to those presented in Tables 6A-6B, and which may be referred to herein by a common name, short name, and/or a gene name.

Table 6A Tumor Antigens

| Protein Tumor Antigen | Gene Name(s) | UniProtKB ID No. |
|---|---|---|
| Alpha-N-acetylgalactosaminide alpha- | ST6GALNAC6; | Q969X2 |
| 2,6-sialyltransferase 6 | CA19-9 | |
| 5,6-dihydroxyindole-2-carboxylic acid | TYRP1; gp75 | P17643 |
| oxidase | | |
| Activated leukocyte cell adhesion | ALCAM; CD166 | Q13740 |
| molecule | | |
| Alpha-1,4-N- | A4GNT | Q9UNA3 |
| acetylglucosaminyltransferase | | |
| B melanoma antigen 1 | BAGE; CT2.1 | Q13072 |
| Basigin | BSG; CD147 | P35613 |
| B-cell antigen receptor complex- | CD79A | P11912 |
| associated protein alpha chain | | |
| B-cell antigen receptor complex- | CD79B | P40259 |
| associated protein beta chain | | |
| B-cell receptor CD22 | BL-CAM; CD22 | P20273 |
| B-lymphocyte antigen CD19 | CD19 | P15391 |
| B-lymphocyte antigen CD20 | MS4A1; CD20 | P11836 |
| Bone marrow stromal antigen 2 | BST2; CD317 | Q10589 |
| Campath-1 antigen | CD52 | P31358 |
| Carbonic anhydrase 14 | CA14 | Q9ULX7 |
| Carboxypeptidase M | CPM | P14384 |
| Carcinoembryonic antigen-related cell | CEACAM5; CD66e | P06731 |
| adhesion molecule 5 | | |
| Carcinoembryonic antigen-related cell | CEACAM6; CD66c | P40199 |
| adhesion molecule 6 | | |
| Catenin beta-1 | CTNNB1; beta- | P35222 |
| | catenin | |
| CD27 antigen | CD27 | P26842 |
| CD276 antigen | CD276; B7-H3 | Q5ZPR3 |
| CD40 ligand | CD40LG; CD154 | P29965 |
| Cell-surface A33 antigen | GPA33 | Q99795 |
| Chondroitin sulfate proteoglycan 4 | CSPG4 | Q6UVK1 |
| C-type lectin domain family member 4 | CLEC4C; BDCA2; | Q8WTT0 |
| C | CD303 | |
| Cyclin-dependent kinase 4 | CDK4 | P11802 |
| Cytotoxic T-lymphocyte protein 4 | CTLA4 | P16410 |
| Disintegrin and metalloproteinase | ADAM-9 | Q13443 |
| domain-containing protein 9 | | |
| Ephrin type-A receptor 2 | EPHA2 | P29317 |
| Epidermal growth factor receptor | EGFR; ERBB1; | P00533 |
| | HER1 | |
| Epithelial cell adhesion molecule | EPCAM; CD326 | P16422 |
| G antigen 1 | GAGE1; CT4.1 | Q13065 |
| G antigen 2A | GAGE2A | Q6NT46 |
| G antigen 2B/C | GAGE2B | Q13066 |
| G antigen 2D | GAGE2D | Q9UEU5 |
| G antigen 2E | GAGE2E | Q4V326 |
| G2/mitotic-specific cyclin-B1 | CCNB1 | P14635 |
| GDP-L-fucose synthase | TSTA3 | Q13630 |
| Glutamate carboxypeptidase 2 | FOLH1; PSMA | Q04609 |
| Hyaluronidase-2 | HYLA2; LUCA2 | Q12891 |
| Inactive tyrosine-protein kinase | ROR1, NTRKR1 | Q01973 |
| transmembrane receptor ROR1 | | |
| Integrin alpha-E | ITGAE; CD103 | P38570 |
| Integrin beta-6 | ITGB6 | P18564 |
| Interleukin-13 receptor subunit alpha-2 | IL13RA2; CD213a2 | Q14627 |
| (subunit of CD123, interleukin -3 | | |
| receptor) | | |
| Interleukin-2 receptor subunit alpha | IL2RA: CD25 | P01589 |
| Junctional adhesion molecule C | JAM3 | Q9BX67 |
| Keratin, type II cytoskeletal 8 | CK-8; KRT8 | P05787 |
| Lactadherin | MFGE8 | Q08431 |
| Low-affinity immunoglobulin epsilon | FCER2; CD23 | P06734 |
| Fc receptor | | |
| Melanocyte protein PMEL | PMEL; gp100 | P40967 |
| Melanoma antigen recognized by T- | MLANA; MART1 | Q16655 |
| cells 1 | | |
| Melanoma-associated antigen 1 | MAGEA1; MAGE1 | P43355 |
| Melanoma-associated antigen 3 | MAGEA3; MAGE3 | P43357 |
| Melanotransferrin | MELTF; MAAp97; | P08582 |
| | CD228 | |
| Membrane cofactor protein | CD46 | P15529 |
| Mesothelin | MSLN | Q13421 |
| Mucin-1 | MUC1; PEM | P15941 |
| Mucin-16 | MUC16; CA-125 | Q8WXI7 |
| Myeloid cell-surface antigen CD33 | CD33 | P20138 |
| Neural cell adhesion molecule 1 | NCAM1; CD56 | P13591 |
| Oncostatin-M | OSM | P13725 |
| Oncostatin-M-specific receptor subunit | OSMR; IL31RB | Q99650 |
| beta | | |
| Platelet glycoprotein 4 | CD36 | P16671 |
| Programmed cell death 1 ligand 1 | CD274 | Q9NZQ7 |
| Prosaposin receptor GPR37 | GPR37 | O15354 |
| Prostate-specific antigen | KLK3; PSA | P07288 |
| Prostatic acid phosphatase | ACPP | P15309 |
| Protein PML | PML; TRIM19; Myl | P29590 |
| PWWP domain-containing DNA repair | PWWP3A; MUM1 | Q2TAK8 |
| factor 3A | | |
| Receptor tyrosine-protein kinase erbB-2 | ERBB2; HER2; | P04626 |
| | CD340 | |
| Receptor tyrosine-protein kinase erbB-3 | ERBB3; HER3 | P21860 |
| Receptor tyrosine-protein kinase erbB-4 | ERBB4; HER4 | Q15303 |
| Receptor-type tyrosine-protein | PTPRC; CD45 | P08575 |
| phosphatase C | | |
| T-cell surface glycoprotein CD5 | CD5 | P06127 |
| T-cell-specific surface glycoprotein | CD28 | P10747 |
| CD28 | | |
| Transferrin receptor protein 1 | TFRC; CD71 | P02786 |
| Transmembrane 4 L6 family member 1 | TM4SF1; TAAL6 | P30408 |
| Trophoblast glycoprotein | TPBG; 5T4 | Q13641 |
| Tumor necrosis factor receptor | TNFRSF10B; DR5; | O14763 |
| superfamily member 10B | CD262 | |
| Tumor necrosis factor receptor | TNFRSF1A; TNFR1; | P19438 |
| superfamily member 1A | CD120a | |
| Tumor necrosis factor receptor | TNFRSF1B; TNFR2; | P20333 |
| superfamily member 1B | CD120b | |
| Tumor necrosis factor receptor | LTBR; TNFR3 | P36941 |
| superfamily member 3 | | |
| Tumor necrosis factor receptor | CD40 | P25942 |
| superfamily member 5 | | |
| Tumor necrosis factor receptor | TNFR6; Apo-1; Fas; | P25445 |
| superfamily member 6 | CD95 | |
| Ubiquitin-conjugating enzyme E2 K | UBE2K | P61086 |
| Ubiquitin-protein ligase E3A | UBE3A | Q05086 |
| Vascular endothelial growth factor A | VEGFA | P15692 |
| Vascular endothelial growth factor B | VEGFB | P49765 |
| Vascular endothelial growth factor | FLT1; VEGFR1 | P17948 |
| receptor 1 | | |
| Vascular endothelial growth factor | KDR; VEGFR2; | P35968 |
| receptor 2 | CD309 | |
| Vascular endothelial growth factor | FLT4; VEGFR3 | P35916 |
| receptor 3 | | |
| Zinc finger protein 354C | ZNF354C; KID3 | Q86Y25 |

Table 6B Tumor Antigens

| Tumor Antigen | Citation(s) |
|---|---|
| 3-fucosyl-N- | Gooi, H. C. (1983), “Marker Of Peripheral Blood |
| acetyllactosamine | Granulocytes And Monocytes Of Man Recognized By |
| | Two Monoclonal Antibodies VEP8 And VEP9 Involves |
| | The Trisaccharide 3-Fucosyl-N-Acetyllactosamine,” |
| | Eur. J. Immuno. 13(4): 306-12. |
| Blood group A | Gooi, H. C., et al. (1983) “Monoclonal Antibody |
| antigen | Reactive With The Human Epidermal Growth Factor |
| | Receptor Recognizes The Blood Group A Antigen,” |
| | Biosci. Rep. 3(11): 1045-52. |
| Difucosyl type | Dohi, T. et al. (1989) “Immunohistochemical Study Of |
| 1 chain (Aleb) | Carbohydrate Antigen Expression In Gastric |
| Difucosyl type | Carcinoma,” Gastroenterol Jpn. 24(3): 239-45; |
| 2 chain (ALey) | Yazawa, S. et al. (1993), “Aberrant alpha1→2 |
| | Fucosyltransferases Found in Human Colorectal |
| | Carcinoma Involved in the Accumulation of Leb and Y |
| | Antigens in Colorectal Tumors,” Jpn. J. Cancer Res. |
| | 84: 989-995 |
| Ganglioside | Nudelman, E. et al. (1982) “Characterization Of A |
| antigen 4.2 | Human Melanoma-Associated Ganglioside Antigen |
| | Defined By Monoclonal Antibody, 4.2,” J. Biol. Chem. |
| | 257(21): 12752-6. |
| Ganglioside | Levine, J. M., et al. (1984) “The D1.1 Antigen: A Cell- |
| antigen D1.1 | Surface Marker For Germinal Cells Of The Central |
| | Nervous System,” J. Neurosci. 4(3): 820-31. |
| Gangliosides | Krengel, U. and Bousquet P. A. (2014), “Molecular |
| GD2/GD3/ | Recognition of Gangliosides and Their Potential for |
| GM2/GM3 | Cancer Immunotherapies,” Front. Immuno. 5(325): 1- |
| | 11. |
| Lactosylceramide | Symington, F. W. (1984) “Monoclonal Antibody |
| | Specific for Lactosylceramide,” J. Biol. Chem. |
| | 259(9): 6008-6012. |
| Rh antigens | Avent, N. D. and Reid, M. E. (2000) “The Rh Blood |
| (D, C, c, E or e) | Group System: A Review,” Blood 95: 375-387. |
| Sialyl-Tn | Holmberg, L. A. (2001) “Theratope Vaccine (STn- |
| | KLH),” Expert Opin. Biol. Ther. 1(5): 881-91. |

B. TA-Binding Domains and Molecules

A number of TA-Binding Molecules are known in the art or can be generated using well-known methods, including those described herein. TA-Binding Molecules may be monospecific, or bispecific. Representative TA-Binding Molecules that comprise TA-Binding Domains, and whose sequences or polypeptide chains may thus be employed in the construction of, or used as, TA-Binding Molecules of the invention (e.g., ADCC-Enhanced TA-Binding Molecules), are listed in Table 7. The CDRs, VH and VL Domains for several TA-Binding Molecules are presented below.

TABLE 7
TA-Binding Molecules

| Antibody Name | Tumor Antigen(s) | Therapeutic Target Application |
|---|---|---|
| Abagovomab | CA-125 | Ovarian Cancer |
| Adecatumumab | Epcam | Prostate And Breast Cancer |
| Afutuzumab | CD20 | Lymphoma |
| Alacizumab | VEGFR2 | Cancer |
| Altumomab | CEA | Colorectal Cancer |
| Amatuximab | Mesothelin | Cancer |
| Anatumomab | TAG-72 | Non-Small Cell Lung Carcinoma |
| Mafenatox | | |
| Anifrolumab | Interferon A/B | Systemic Lupus Erythematosus |
| | Receptor | |
| Anrukinzumab | IL-13 | Cancer |
| Apolizumab | HLA-DR | Hematological Cancers |
| Arcitumomab | CEA | Gastrointestinal Cancer |
| Atinumab | RTN4 | Cancer |
| Bectumomab | CD22 | Non-Hodgkin's Lymphoma (Detection) |
| Belimumab | BAFF | Non-Hodgkin Lymphoma |
| Bevacizumab | VEGF-A | Metastatic Cancer, Retinopathy Of |
| | | Prematurity |
| Bivatuzumab | CD44 V6 | Squamous Cell Carcinoma |
| Blinatumomab | CD19 | Cancer |
| Brentuximab | CD30 (TNFRSF8) | Hematologic Cancers |
| Cantuzumab | MUC1 | Cancers |
| Cantuzumab | Mucin Canag | Colorectal Cancer |
| Mertansine | | |
| Caplacizumab | VWF | Cancers |
| Capromab | Prostatic | Prostate Cancer (Detection) |
| | Carcinoma Cells | |
| Carlumab | MCP-1 | Oncology/Immune Indications |
| Catumaxomab | Epcam, CD3 | Ovarian Cancer, Malignant Ascites, |
| | | Gastric Cancer |
| Cetuximab | EGFR | Metastatic Colorectal Cancer And |
| | | Head And Neck Cancer |
| Citatuzumab | Epcam | Ovarian Cancer And Other Solid |
| | | Tumors |
| Cixutumumab | IGF-1 Receptor | Solid Tumors |
| Clivatuzumab | MUC1 | Pancreatic Cancer |
| Conatumumab | TRAIL-R2 | Cancer |
| Dacetuzumab | CD40 | Hematologic Cancers |
| Dalotuzumab | Insulin-Like | Cancer |
| | Growth Factor I | |
| | Receptor | |
| Daratumumab | CD38 | Cancer |
| Demcizumab | DLL4 | Cancer |
| Denintuzumab | CD19 | Acute Lymphoblastic Leukemia And |
| | | B-Cell Non-Hodgkin Lymphoma |
| Detumomab | B-Lymphoma Cell | Lymphoma |
| Drozitumab | DR5 | Cancer |
| Duligotumab | HER3 | Cancer |
| Dusigitumab | ILGF2 | Cancer |
| Ecromeximab | GD3 Ganglioside | Malignant Melanoma |
| Edrecolomab | Epcam | Colorectal Carcinoma |
| Elotuzumab | SLAMF7 | Multiple Myeloma |
| Elsilimomab | IL-6 | Cancer |
| Enavatuzumab | TWEAK Receptor | Cancer |
| Enlimomab | ICAM-1 (CD54) | Cancer |
| Enoticumab | DLL4 | Cancer |
| Ensituximab | 5AC | Cancer |
| Epitumomab | Episialin | Cancer |
| Cituxetan | | |
| Epratuzumab | CD22 | Cancer, SLE |
| Ertumaxomab | HER2, CD3 | Breast Cancer |
| Etaracizumab | Integrin Aᵥβ₃ | Melanoma, Prostate Cancer, Ovarian |
| | | Cancer |
| Faralimomab | Interferon Receptor | Cancer |
| Farletuzumab | Folate Receptor 1 | Ovarian Cancer |
| Fasinumab | HNGF | Cancer |
| Fbta05 (Bi20) | CD20 | Chronic Lymphocytic Leukemia |
| Ficlatuzumab | HGF | Cancer |
| Figitumumab | IGF-1 Receptor | Adrenocortical Carcinoma, Non-Small |
| | | Cell Lung Carcinoma |
| Flanvotumab | TYRP1 | Melanoma |
| | (Glycoprotein 75) | |
| Flotetuzumab | CD123 | Acute Myeloid Leukemia |
| Fresolimumab | TGF-B | Cancer |
| Futuximab | EGFR | Cancer |
| Galiximab | CD80 | B-Cell Lymphoma |
| Ganitumab | IGF-I | Cancer |
| Gemtuzumab | CD33 | Acute Myelogenous Leukemia |
| Ozogamicin | | |
| Girentuximab | Carbonic | Clear Cell Renal Cell Carcinoma |
| | Anhydrase 9 (CA- | |
| | IX) | |
| Glembatumumab | GPNMB | Melanoma, Breast Cancer |
| Vedotin | | |
| Ibritumomab | CD20 | Non-Hodgkin's Lymphoma |
| Tiuxetan | | |
| Icrucumab | VEGFR-1 | Cancer |
| Imgatuzumab | EGFR | Cancer |
| Inclacumab | Selectin P | Cancer |
| Indatuximab | SDC1 | Cancer |
| Ravtansine | | |
| Inotuzumab | CD22 | Cancer |
| Ozogamicin | | |
| Intetumumab | CD51 | Solid Tumors (Prostate Cancer, |
| | | Melanoma) |
| Ipilimumab | CD152 | Melanoma |
| Iratumumab | CD30 (TNFRSF8) | Hodgkin's Lymphoma |
| Itolizumab | CD6 | Cancer |
| Labetuzumab | CEA | Colorectal Cancer |
| Lampalizumab | CFD | Cancer |
| Lebrikizumab | Il-13 | Hodgkin's Lymphoma |
| Lexatumumab | TRAIL-R2 | Cancer |
| Ligelizumab | IGHE | Cancer |
| Lintuzumab | CD33 | Cancer |
| Lirilumab | KIR2D | Cancer |
| Lorvotuzumab | CD56 | Cancer |
| Lucatumumab | CD40 | Multiple Myeloma, Non-Hodgkin's |
| | | Lymphoma, Hodgkin's Lymphoma |
| Lumiliximab | CD23 | Chronic Lymphocytic Leukemia |
| Mapatumumab | TRAIL-R1 | Cancer |
| Matuzumab | EGFR | Colorectal, Lung And Stomach Cancer |
| Milatuzumab | CD74 | Multiple Myeloma And Other |
| | | Hematological Malignancies |
| Minretumomab | TAG-72 | Cancer |
| Mirzotamab | B7-H3 | Cancer |
| clezutoclax | | |
| Mitumomab | GD3 Ganglioside | Small Cell Lung Carcinoma |
| Mogamulizumab | CCR4 | Cancer |
| Morolimumab | Rhesus Factor | Cancer |
| Moxetumomab | CD22 | Cancer |
| Pasudotox | | |
| Nacolomab | C242 Antigen | Colorectal Cancer |
| Tafenatox | | |
| Namilumab | CSF2 | Cancer |
| Naptumomab | 5T4 | Non-Small Cell Lung Carcinoma, |
| Estafenatox | | Renal Cell Carcinoma |
| Namatumab | RON | Cancer |
| Naxitamab | GD2 | Neuroblastoma, Osteosarcoma |
| Necitumumab | EGFR | Non-Small Cell Lung Carcinoma |
| Nerelimomab | TNF-A | Cancer |
| Nesvacumab | Angiopoietin 2 | Cancer |
| Nimotuzumab | EGFR | Squamous Cell Carcinoma, Head And |
| | | Neck Cancer, Nasopharyngeal Cancer, |
| | | Glioma |
| Nofetumomab | Undetermined | Cancer |
| Merpentan | | |
| Ocaratuzumab | CD20 | Cancer |
| Ofatumumab | CD20 | Chronic Lymphocytic Leukemia |
| Olaratumab | PDGF-R A | Cancer |
| Olokizumab | IL6 | Cancer |
| Omburtamab | B7-H3 | Neuroblastoma, Sarcoma, Metastatic |
| | | Brain Cancers |
| Onartuzumab | Human Scatter | Cancer |
| | Factor Receptor | |
| | Kinase | |
| Ontuxizumab | TEM1 | Cancer |
| Oportuzumab | Epcam | Cancer |
| Monatox | | |
| Oregovomab | CA-125 | Ovarian Cancer |
| Orticumab | Oxldl | Cancer |
| Otlertuzumab | CD37 | Cancer |
| Panitumumab | EGFR | Colorectal Cancer |
| Pankomab | Tumor-Specific | Ovarian Cancer |
| | Glycosylation Of | |
| | MUC1 | |
| Parsatuzumab | EGFL7 | Cancer |
| Patritumab | HER3 | Cancer |
| Pemtumomab | MUC1 | Cancer |
| Perakizumab | IL17A | Arthritis |
| Pertuzumab | HER2 | Cancer |
| Pinatuzumab | CD22 | Cancer |
| Vedotin | | |
| Pintumomab | Adenocarcinoma | Adenocarcinoma |
| | Antigen | |
| Placulumab | Human TNF | Cancer |
| Polatuzumab | CD79B | Cancer |
| Vedotin | | |
| Pritoxaximab | E. Coli Shiga Toxin | Cancer |
| | Type-1 | |
| Pritumumab | Vimentin | Brain Cancer |
| Quilizumab | IGHE | Cancer |
| Racotumomab | N- | Cancer |
| | Glycolylneuraminic | |
| | Acid | |
| Radretumab | Fibronectin Extra | Cancer |
| | Domain-B | |
| Ramucirumab | VEGFR2 | Solid Tumors |
| Rilotumumab | HGF | Solid Tumors |
| Rituximab | CD20 | Lymphomas, Leukemias, Some |
| | | Autoimmune Disorders |
| Robatumumab | IGF-1 Receptor | Cancer |
| Roledumab | RHD | Cancer |
| Samalizumab | CD200 | Cancer |
| Satumomab | TAG-72 | Cancer |
| Pendetide | | |
| Seribantumab | ERBB3 | Cancer |
| Sibrotuzumab | FAP | Cancer |
| Siltuximab | IL-6 | Cancer |
| Solitomab | Epcam | Cancer |
| Sontuzumab | Episialin | Cancer |
| Tabalumab | BAFF | B-Cell Cancers |
| Tacatuzumab | Alpha-Fetoprotein | Cancer |
| Tetraxetan | | |
| Taplitumomab | CD19 | Cancer |
| Paptox | | |
| Telimomab | Undetermined | Cancer |
| Tenatumomab | Tenascin C | Cancer |
| Teneliximab | CD40 | Cancer |
| Teprotumumab | CD221 | Hematologic Tumors |
| Ticilimumab | CTLA-4 | Cancer |
| Tigatuzumab | TRAIL-R2 | Cancer |
| Tositumomab | CD20 | Follicular Lymphoma |
| Tovetumab | CD140a | Cancer |
| Trastuzumab | HER2 | Breast Cancer |
| Trbs07 (Ektomab) | Gd2 | Melanoma |
| Tremelimumab | CTLA-4 | Cancer |
| Tucotuzumab | Epcam | Cancer |
| Celmoleukin | | |
| Ublituximab | MS4A1 | Cancer |
| Urelumab | 4-1BB | Cancer |
| Vadastuximab | CD33 | Acute Myeloid Leukemia |
| Vantictumab | Frizzled Receptor | Cancer |
| Vapaliximab | AOC3 (VAP-1) | Cancer |
| Vatelizumab | ITGA2 | Cancer |
| Veltuzumab | CD20 | Non-Hodgkin's Lymphoma |
| Vesencumab | NRP1 | Cancer |
| Volociximab | Integrin A5β1 | Solid Tumors |
| Vorsetuzumab | CD70 | Cancer |
| Votumumab | Tumor Antigen | Colorectal Tumors |
| | CTAA16.88 | |
| Zalutumumab | EGFR | Squamous Cell Carcinoma Of The |
| | | Head And Neck |
| Zatuximab | HER1 | Cancer |
| Ziralimumab | CD147 | Cancer |
| Zolbetuximab | Cldn18.2 | Gastrointestinal Adenocarcinomas And |
| | | Pancreatic Tumor |

In one embodiment, the invention relates to TA-Binding Molecules that comprise the CDR Domains (or the VL and VH Domains) of any of the TA-Binding Molecules listed in Table 7. In an additional embodiment, the invention uses any of the TA-Binding Molecules listed in Table 7, or as provided below. In an alternative embodiment, the invention relates to ADCC-Enhanced TA-Binding Molecules that comprise the CDR Domains (or the VL and VH Domains) of any of the antibodies listed in Table 7. Particular examples of ADCC-Enhanced TA-Binding Molecules are provided below.

In certain embodiments the TA-Binding molecule binds the HER2 TA (“HER2-Binding Molecule”). In one embodiment, a HER2-Binding Molecule of the present invention is an anti-HER2 antibody. Antibodies that bind human HER2 include “margetuximab,” “trastuzumab,” and “pertuzumab.” Margetuximab (also known as MGAH22; CAS Reg No. 1350624-75-7, KEGG D10446, see for example, U.S. Pat. No. 8,802,093) is an Fc-optimized monoclonal antibody that binds to HER2 and mediates enhanced ADCC activity. The sequence of margetuximab is provided below. Trastuzumab (also known as rhuMAB4D5, and marketed as HERCEFMN®; CAS Reg No 180288-69-1; see, U.S. Pat. No. 5,821,337) is a humanized antibody, having IgG1/kappa constant regions. The amino acid sequence of trastuzumab is found in WHO Drug Information, 2011, Recommended INN: List 65, 25(1):89-90 for trastuzumab emtansine) Pertuzumab (also known as rhuMAB2C4, and marketed as PERJETA™; CAS Reg No 380610-27-5; see for example, PCT Publication No. WO 2001/000245) is another humanized antibody having IgG1/kappa constant regions. The amino acid sequence of the Fab domain of pertuzumab is found in Protein Data Bank Accession No. 117i). Antibody “8H11” is a murine anti-HER2 monoclonal antibody that binds an epitope of HER2 that is distinct from the epitope recognized by margetuximab, trastuzumab and pertuzumab (PCT Publication No. WO 2001/036005). Humanized variants of Antibody 8H11 (designated “hHER2 MAB-1”) been described (see for example, WO 2018/156740) and representative humanized VH and VL Domains are provided below. In addition to the above-identified HER2-Binding Molecules, the invention contemplates the use of any of the following HER2-Binding Molecules: 1.44.1; 1.140; 1.43; 1.14.1; 1.100.1; 1.96; 1.18.1; 1.20; 1.39; 1.24; and 1.71.3 (disclosed in U.S. Pat. Nos. 8,350,011; 8,858,942; and PCT Publication No. WO 2008/019290); F5 and C1 (disclosed in U.S. Pat. Nos. 7,892,554; 8,173,424; 8,974,792; and PCT Publication No. WO 99/55367); and also the HER2-Binding Molecules of US Patent Publication 2011/0097323, 2013/017114, 2014/0328836, 2016/0130360 and 2016/0257761, and PCT Patent Publication WO2011/147986.

In certain embodiments the TA-Binding molecule binds the B7-H3 TA (“B7-H3-Binding Molecule”). In one embodiment, a B7-H3-Binding Molecule of the present invention is an anti-B7-H3 antibody. Antibodies that bind human B7-H3 include “enoblituzumab,” and “omburtamab,” and “mirzotamab.” Enoblituzumab (also known as MGAH22; CAS Reg No. 1350624-75-7, KEGG D11752, see for example, U.S. Pat. No. 8,802,093) is an Fc-optimized monoclonal antibody that binds to HER2 and mediates enhanced ADCC activity. The sequence of margetuximab is provided below. Omburtamab (also known as 8H9; CAS Reg No. 1895083-75-6, see for example, U.S. Pat. No. 7,737,258) is a murine monoclonal antibody. The amino acid sequence of omburtamab is found in WHO Drug Information 2018, Proposed INN: List 119, 32(2):339-340). Humanized versions of 8H9 are disclosed in WO 2016/033225. Mirzotamab clezutoclax (also known as ABBV-155; CAS Reg No. 2229859-12-3, see for example WO 2017/214322) is a humanized antibody having IgG1/kappa constant regions. The amino acid sequence of mirzotamab is found in WHO Drug Information 2019, Proposed INN: List 121, 33(2): 294-6). In addition to the above-identified B7-H3-Binding Molecules, the invention contemplates the use of any of the following B7-H3-Binding Molecules: BRCA84D, BRCA69D and PRCA157 (disclosed in WO2011109400); L7, L8, L11, M30, and M31 (disclosed in US2013/0078234), hmAb-C, and B7-H3 Antibody hmAb-D (disclosed in WO 2017/180813).

C. ADCC Enhanced TA-Binding Molecules

The present invention specifically contemplates compositions and methods that include or employ margetuximab and:
(1) a PD-1×LAG-3 bispecific molecule;
(2) a monospecific PD-1-Binding Molecule, and a monospecific LAG-3-Binding Molecule;
(3) a PD-L1×LAG-3 bispecific molecule; or
(4) a monospecific PD-L1-Binding Molecule, and a monospecific LAG-3-Binding Molecule,
   wherein such monospecific binding molecule is an intact antibody, and such bispecific molecule is a diabody or a bispecific antibody.

1. Margetuximab

Margetuximab comprises a variant human Fc Domain that exhibits increased affinity to the CD16A receptor. The Light Chain of the antibody (IgG Kappa) has been modified (N65S; double underlined below) to delete an N-linked glycosylation site.

The VL Domain of margetuximab has the amino acid sequence of SEQ ID NO:61:

DIVMTQSHKE MSTSVGDRVS ITC [symbol] [symbol] WYQQKP
GHSPKLLIY [symbol] [symbol] GVPD RFTGSRSGTD FTFTISSVQA
EDLAVYYC [symbol] [symbol] FGG GTKVEIK

The CDR Domains of the VL Domain of margetuximab are:

CDR_{L}1 SEQ ID NO: 62:
KASQDVNTAVA
CDR_{L}2 SEQ ID NO: 63:
SASFRYT
and
CDR_{L}3 SEQ ID NO: 64:
QQHYTTPPT.

The Light Chain of marueiuximab has the amino acid sequence of SEQ ID NO:65:

DIVMTQSHKE MSTSVGDRVS ITCKASQDVN TAVAWYQQKP
GHSPKLLIYS ASFRYTGVPD RFTG [symbol] RSGTD FTFTISSVQA
EDLAVYYCQQ HYTTPPTFGG GTKVEIKRTV AAPSVFIFPP
SDEQLKSGTA SVVCLLNNFY PREAKVQWKV DNALQSGNSQ
ESVTEQDSKD STYSLSSTLT LSKADYEKHK VYACEVTHQG
LSSPVTKSFN RGEC

The VH Domain of margetuximab has the amino acid sequence of SEQ ID NO:66:

QVQLQQSGPE LVKPGASLKL SCTASGFNIK [symbol] WVKQR
PEQGLEWIG [symbol] [symbol] [symbol] KATI TADTSSNTAY
LQVSRLTSED TAVYYCSR [symbol] [symbol] W GQGASVTVSS

The CDR Domains of the VH Domain of margetuximab are:

CDR_{H}1 SEQ ID NO: 67:
DTYIH
CDR_{H}2 SEQ ID NO: 68:
RIYPTNGYTRYDPKFQD
and
CDR_{H}3 SEQ ID NO: 69
WGGDGFYAMDY.

The Heavy Chain of margetuximab comprises the FcMT2 ADCC-Enhanced Fc Domain (comprising L235V, F243L, R292P, Y300L, and P396L substitutions; underlined) has the amino acid sequence of SEQ ID NO:70:

QVQLQQSGPE LVKPGASLKL SCTASGFNIK DTYIHWVKQR
PEQGLEWIGR TYPTNGYTRY DPKFQDKATI TADTSSNTAY
LQVSRLTSED TAVYYCSRWG GDGFYAMDYW GQGASVTVSS
ASTKGPSVFP LAPSSKSTSG GTAALGCLVK DYFPEPVTVS
WNSGALTSGV HTFPAVLQSS GLYSLSSVVT VPSSSLGTQT
YICNVNHKPS NTKVDKRVEP KSCDKTHTCP PCPAPEL [symbol] GG
PSVFL [symbol] PPKP KDTLMISRTP EVTCVVVDVS HEDPEVKFNW
YVDGVEVHNA KTKP [symbol] EEQYN ST [symbol] RVVSVLT VLHQDWLNGK
EYKCKVSNKA LPAPIEKTIS KAKGQPREPQ VYTLPPSRDE
LTKNQVSLTC LVKGFYPSDI AVEWESNGQP ENNYKTTP [symbol] V
LDSDGSFFLY SKLTVDKSRW QQGNVFSCSV MHEALHNHYT
QKSLSLSPGK

A variant of the Heavy Chain of margetuximab comprises the FcMT1 ADCC-Enhanced Fc Domain (comprising F243L, R292P, Y300L, V305I, and P396L substitutions; see SEQ ID NO:16). Another variant of the Heavy Chain of margetuximab comprises the FcMT3 ADCC-Enhanced Fc Domain (comprising F243L, R292P, and Y300L substitutions; see SEQ ID NO:18).

The present invention specifically contemplates compositions and methods that include or employ enoblituzumab and:
(1) a PD-1×LAG-3 bispecific molecule;
(2) a monospecific PD-1-Binding Molecule, and a monospecific LAG-3-Binding Molecule;
(3) a PD-L1×LAG-3 bispecific molecule; or
(4) a monospecific PD-L1-Binding Molecule, and a monospecific LAG-3-Binding Molecule,
   wherein such monospecific Antibody-Based Molecule is an intact antibody, and such bispecific Antibody-Based Molecule is a diabody, or a bispecific antibody.

2. Enoblituzumab

The VL Domain of enoblituzumab has the amino acid sequence of SEQ ID NO:71:

DIQLTQSPSF LSASVGDRVT ITC [symbol] [symbol] WYQQKP
GKAPKALIY [symbol] [symbol] GVPS RFSGSGSGTD FTLTISSLQP
EDFATYYC [symbol] [symbol] GQ GTKLEIK

The CDR Domains of the VL Domain of enoblituzumab are:

CDR_{L}1 SEQ ID NO: 72:
KASQNVDTNVA
CDR_{L}2 SEQ ID NO: 73:
SASYRYS
and
CDR_{L}3 SEQ ID NO: 74:
QQYNNYPFT.

The Light Chain of enoblituzumab has the amino acid sequence of SEQ ID NO:75:

DIQLTQSPSF LSASVGDRVT ITCKASQNVD TNVAWYQQKP
GKAPKALIYS ASYRYSGVPS RFSGSGSGTD FTLTISSLQP
EDEATYYCQQ YNNYPFTFGQ GTKLEIKRTV AAPSVFIFPP
SDEQLKSGTA SVVCLLNNFY PREAKVQWKV DNALQSGNSQ
ESVTEQDSKD STYSLSSTLT LSKADYEKHK VYACEVTHQG
LSSPVTKSFN RGEC

The VH Domain of enoblituzumab has the amino acid sequence of SEQ II NO:76:

EVQLVESGGG LVQPGGSLRL SCAASGFTFS [symbol] WVRQA
PGKGLEWVA [symbol] [symbol] [symbol] RFTI SRDNAKNSLY
LQMNSLRDED TAVYYCGR [symbol] [symbol] [symbol] WGQGTTVTV
SSASTKGPSV FPLAPSSKST SGGTAALGCL VKDYFPEPVT
VSWNSGALTS GVHTFPAVLQ SSGLYSLSSV VTVPSSSLGT
QTYICNVNHK PSNTKVDKRV

The CDR Domains of the VH Domain of enoblituzumab are:

CDR_{H}1 SEQ ID NO: 77:
SFGMH
CDR_{H}2 SEQ ID NO: 78:
YISSDSSAIYYADTVKG
and
CDR_{H}3 SEQ ID NO: 79:
GRENIYYGSRLDY

The Heavy Chain of enoblituzumab comprises the FcMT2 ADCC-Enhanced Fc Domain (comprising L235V, F243L, R292P, Y300L, and P396L substitutions; underlined) and has the amino acid sequence of SEQ ID NO:80:

EVQLVESGGG LVQPGGSLRL SCAASGFTFS SFGMHWVRQA
PGKGLEWVAY ISSDSSAIYY ADTVKGRFTI SRDNAKNSLY
LQMNSLRDED TAVYYCGRGR ENIYYGSRLD YWGQGTTVTV
SSASTKGPSV FPLAPSSKST SGGTAALGCL VKDYFPEPVT
VSWNSGALTS GVHTFPAVLQ SSGLYSLSSV VTVPSSSLGT
QTYICNVNHK PSNTKVDKRV EPKSCDKTHT CPPCPAPEL [symbol]
GGPSVFL [symbol] PP KPKDTLMISR TPEVTCVVVD VSHEDPEVKF
NWYVDGVEVH NAKTKP [symbol] EEQ YNST [symbol] RVVSV LTVLHQDWLN
GKEYKCKVSN KALPAPIEKT ISKAKGQPRE PQVYTLPPSR
EEMTKNQVSL TCLVKGFYPS DIAVEWESNG QPENNYKTTP [symbol]
VLDSDGSEE LYSKLTVDKS RWQQGNVFSC SVMHEALHNH
YTQKSLSLSP GK

A variant of the Heavy Chain of enoblituzumab comprises the FcMT1 ADCC-Enhanced Fc Domain (comprising F243L, R292P, Y300L, V305I, and P396L substitutions; see SEQ ID NO:16). Another variant of the Heavy Chain of enoblituzumab comprises the FcMT3 ADCC-Enhanced Fc Domain (comprising F243L, R292P, and Y300L substitutions; see SEQ ID NO:18).

3. Other ADCC-Enhanced Fc TA-Binding Molecules

The present invention specifically contemplates compositions and methods that include or employ an ADCC-Enhanced TA-Binding Molecule and:
(1) PD-1×LAG-3 bispecific molecule;
(2) a monospecific PD-1-Binding Molecule, and a monospecific LAG-3-Binding Molecule;
(3) a PD-L1×LAG-3 bispecific molecule; or
(4) a monospecific PD-L1-Binding Molecule, and a monospecific LAG-3-Binding Molecule,
   wherein such monospecific binding molecule is an intact antibody, and such bispecific molecule is a diabody, or a bispecific antibody.

In one embodiment, the invention relates to ADCC-Enhanced TA-Binding molecules that comprise a TA-Binding Domain that immunospecifically binds any of the TAs listed in Tables 6A-6B.

In one embodiment, the invention relates to ADCC-Enhanced TA-Binding Molecules that comprise the CDR Domains (or the VL and VH Domains) of any of the antibodies listed in Table 7. Such molecules may comprise an enhanced ADCC-Enhanced Fc Domain as provided herein, or as known in the art.

The present invention specifically contemplates compositions and methods that include or employ other TA-Binding Molecules that comprise an enhanced ADCC-Enhanced Fc Domain including, but not limited to: Obinutuzumab (KEGG D0932; Marcus, R. et al. (2017) “Obinutuzumab for the First-Line Treatment of Follicular Lymphoma,” N. Engl. J. Med. 377(14):1331-1344) and BAT4306F (Yu, J.-C. et al. (2018) “Abstract 3823: Bat4306f, An Anti-CD20 Antibody Devoid Of Fucose Modification, Demonstrates Enhanced ADCC Effect And Potent In Vivo Efficacy,” Cancer Res. 78:(13 Supplement):3823), which are anti-CD20 antibodies, amivantamab an EGFR-cMET Bispecific Antibody (KEGG D11894; Yun, et al. (2020) “Antitumor Activity of Amivantamab (JNJ-61186372), an EGFR-MET Bispecific Antibody, in Diverse Models of EGFR Exon 20 Insertion-Driven NSCLC” Cancer Discovery DOI: 10.1158/2159-8290.CD-20-0116); and tafasitamab (MOR208)(KEGG D11601; Kellner, C. et al. (2013) “The Fc-Engineered CD19 Antibody MOR208 (Xmab5574) Induces Natural Killer Cell-Mediated Lysis Of Acute Lymphoblastic Leukemia Cells From Pediatric And Adult Patients,” Leukemia 27(7):1595-1598) and obexelimab (KEGG D11496), which are anti-CD19 antibodies.

IV. Methods of Production

The Antibody-Based Molecules of the invention can be may be made recombinantly and expressed using any method known in the art for the production of recombinant proteins. For example, nucleic acids encoding the polypeptide chains of such binding molecules can be constructed, introduced into an expression vector, and expressed in suitable host cells. The binding molecules may be recombinantly produced in bacterial cells (e.g., E coli cells), or eukaryotic cells (e.g., CHO, 293E, COS, NS0 cells). In addition, the binding molecules can be expressed in a yeast cell such as Pichia, or Saccharomyces.

To produce the Antibody-Based Molecules of the invention, one or more polynucleotides encoding the molecule may be constructed, introduced into an expression vector, and then expressed in suitable host cells. Standard molecular biology techniques are used to prepare the recombinant expression vector, transfect the host cells, select for transformants, culture the host cells and recover the molecules (See, for example, the techniques described in Green, M. R. et al., (2012), MOLECULAR CLONING, A LABORATORY MANUAL, 4th Ed., Cold Spring Harbor Laboratory, Cold Spring Harbor, N.Y. and Ausubel et al. eds., (1998,) CURRENT PROTOCOLS IN MOLECULAR BIOLOGY, John Wiley & Sons, NY). The expression vector(s) should have characteristics that permit replication of the vector in the host cell. The vector should also have promoter and signal sequences necessary for expression in the host cells. Such sequences are well known in the art. In addition to the nucleic acid sequence(s) encoding such binding molecules, the recombinant expression vectors may carry additional sequences, such as sequences that regulate replication of the vector in host cells (e.g., origins of replication) and selectable marker genes. Another method that may be employed is to express the gene sequence in plants (e.g., tobacco) or a transgenic animal. Suitable methods useful for expressing such binding molecules recombinantly in plants or milk have been disclosed (see, for example, Peeters et al. (2001) “Production Of Antibodies And Antibody Fragments In Plants,” Vaccine 19:2756; U.S. Pat. No. 5,849,992; and Pollock et al. (1999) “Transgenic Milk As A Method For The Production Of Recombinant Antibodies,” J. Immunol Methods 231:147-157).

Once an Antibody-Based Molecule of the invention has been recombinantly expressed, it may be purified from inside or outside (such as from culture media) of the host cell by any method known in the art for purification of polypeptides or polyproteins. Methods for isolation and purification commonly used for antibody purification (e.g., antibody purification schemes based on antigen selectivity) may be used for the isolation and purification of such molecules, and are not limited to any particular method. For example, by for example, column chromatography, filtration, ultrafiltration, salting out, solvent precipitation, solvent extraction, distillation, immunoprecipitation, SDS-polyacrylamide gel electrophoresis, isoelectric focusing, dialysis, and recrystallization. Chromatography includes, e.g., ion exchange, affinity, particularly by affinity for the specific antigen (optionally after Protein A selection where the Antibody-Based Molecule comprises an Fc Region or a Protein A binding portion thereof), sizing column chromatography, hydrophobic, gel filtration, reverse-phase, and adsorption (Marshak et al. (1996) STRATEGIES FOR PROTEIN PURIFICATION AND CHARACTERIZATION: A LABORATORY COURSE MANUAL. (Eds.), Cold Spring Harbor Laboratory Press, Cold Spring Harbor, N.Y.).

### V. Pharmaceutical Compositions

An Antibody-Based Molecule the invention, for example an antibody that binds a TA (optionally comprises an ADCC-Enhance Fc Domain), an antibody that binds PD-1, an antibody that binds PD-L1, an antibody that binds LAG-3, a PD-1×LAG-3 bispecific molecule, or a PD-L1×LAG-3 bispecific molecule, can be formulated as a composition. The compositions of the invention include bulk drug compositions (e.g., impure or non-sterile compositions) useful in the manufacture of pharmaceutical compositions that are suitable for administration to a subject (e.g., a human patient or other mammal) for the treatment of cancer or other diseases and conditions. Such pharmaceutical compositions comprise one or more Antibody-Based Molecule(s) (e.g., an antibody that binds a TA (optionally comprising an ADCC-Enhance Fc Domain), an antibody that binds PD-1, an antibody that binds PD-L1, an antibody that binds LAG-3, a PD-1×LAG-3 bispecific molecule or a PD-L1×LAG-3 bispecific molecule), and one or more pharmaceutically acceptable carrier(s), and may optionally include one or more additional therapeutic agents. The pharmaceutical compositions may be supplied, for example, as an aqueous solution, a dry lyophilized powder, or water-free concentrate specifically adapted for reconstitution with such a pharmaceutically acceptable carrier, or reconstituted with such a carrier.

As used herein, the term “pharmaceutically acceptable carrier” means a diluent, solvent, dispersion media, antibacterial and antifungal agents, excipient, or vehicle approved by a regulatory agency of the Federal or a state government or listed in the U.S. Pharmacopeia or other generally recognized pharmacopeia as being suitable for administration to animals, and more particularly, to humans. Such pharmaceutical carriers can be sterile liquids, such as water and oils, including those of petroleum, animal, vegetable or synthetic origin. Saline solutions and aqueous dextrose and glycerol solutions can also be employed as liquid carriers, particularly for injectable solutions. The composition, if desired, can also contain minor amounts of wetting or emulsifying agents, or pH buffering agents. These compositions can take the form of solutions, suspensions, emulsion, tablets, pills, capsules, powders, sustained-release formulations and the like.

Generally, the ingredients of compositions of the invention are supplied either separately or mixed together in a dose form, for example, as a dry lyophilized powder or water-free concentrate, or as an aqueous solution in a hermetically sealed container such as a vial, ampoule or sachette indicating the quantity of active agent. Where the composition is to be administered by infusion, it can be dispensed with an infusion bottle containing sterile pharmaceutical grade water or saline. Where the composition is administered by injection, an ampoule of sterile water for injection, saline or other diluent can be provided so that the ingredients may be mixed prior to administration.

### VI. Pharmaceutical Kits

The invention also provides pharmaceutical kits that comprise one or more containers containing a pharmaceutical composition of the invention and instructional material (e.g., a notice, package insert, instruction, etc.). Additionally, one or more other prophylactic or therapeutic agents useful for the treatment of a disease can also be included in the pharmaceutical kit. The containers of such pharmaceutical kits may, for example, comprise one or more hermetically sealed vials, ampoules, sachets, etc., indicating the quantity of active agent contained therein. Where the composition is to be administered by infusion, the container may be an infusion bottle, bag, etc. containing a sterile pharmaceutical-grade solution (e.g., water, saline, a buffer, etc.). Where the composition is to be administered by injection, the pharmaceutical kit may contain an ampoule of sterile water, saline or other diluent for injection, so as to facilitate the mixing of the components of the pharmaceutical kit for administration to a subject (e.g., a human patient or other mammal).

In one embodiment, a pharmaceutical composition of such kit is supplied as a dry sterilized lyophilized powder or water-free concentrate in a hermetically sealed container and can be reconstituted, e.g., with water, saline, or other diluent to the appropriate concentration for administration to a subject. In another embodiment, a pharmaceutical composition of such kit is supplied as an aqueous solution in a hermetically sealed container and can be diluted, e.g., with water, saline, or other diluent, to the appropriate concentration for administration to a subject. The kit can further comprise one or more other prophylactic and/or therapeutic agents useful for the treatment of cancer, in one or more containers; and/or the kit can further comprise one or more cytotoxic antibodies that bind one or more cancer antigens associated with cancer. In certain embodiments, the other prophylactic or therapeutic agent is a chemotherapeutic. In other embodiments, the prophylactic or therapeutic agent is a biological or hormonal therapeutic.

The included instructional material of the pharmaceutical kits of the invention may, for example, be of a content and format prescribed by a governmental agency regulating the manufacture, use or sale of pharmaceuticals or biological products, and may indicate approval by the agency of the manufacture, sale or use of the pharmaceutical composition for human administration and/or for human therapy. The instructional material may, for example provide information relating to the contained dose of the pharmaceutical composition, modes of how it may be administered, etc.

Thus, for example, the included instructional material of the pharmaceutical kits of the invention may instruct that the provided pharmaceutical composition is to be administered in combination with an additional agent which may be provided in the same pharmaceutical kit or in a separate pharmaceutical kit. Such instructional material may instruct that the provided pharmaceutical composition is to be administered once about every 2 weeks, once about every 3 weeks, or more or less often. Such instructional material may instruct that the provided pharmaceutical composition comprises, or is to be reconstituted/diluted to administer a flat dose of about 120 mg, about 300 mg, about 400 mg, about 420 mg, about 600 mg, about 800 mg, or about 840 mg, or more, or to administer a weight-based dose of about 2 mg/kg, about 4 mg/kg, about 6 mg/kg, about 8 mg/kg, about 10 mg/kg, about 15 mg/kg about 18 mg/kg, or more. Such instructional material may instruct that the provided pharmaceutical composition comprises, or is to be reconstituted/diluted to comprise, a single dose, or more than one dose (e.g., 2 doses, 4 doses, 6 doses, 12 doses, 24 doses, etc.). Such included instructional material of the pharmaceutical kits may combine any set of such information (e.g., it may instruct that the provided PD-1×LAG-3 bispecific molecule-containing pharmaceutical composition comprises, or is to be reconstituted/diluted to comprise, a dose of about 400 mg or about 600 mg, and that such dose is to be administered once about every 2 weeks; it may instruct that the provided pharmaceutical composition comprises, or is to be reconstituted to comprise, a dose of about 600 mg or about 800 mg, and that such dose is to be administered once about every 3 weeks; etc., and/or it may instruction that a provided HER2- or B7-H3-Binding Molecule-containing pharmaceutical composition comprises, or is to be reconstituted to comprise, a dose of about 15 mg/kg, and that such dose is to be administered once about every 3 weeks; etc.). Such instructional material may instruct regarding the mode of administration of the included pharmaceutical composition, for example that it is to be administered by intravenous (IV) infusion. The included instructional material of the pharmaceutical kits may instruct regarding the duration or timing of such administration, for example that the included pharmaceutical composition is composition is to be administered by intravenous (IV) infusion over a period of 30-240 minutes, a period of 30-90 minutes, etc.

The included instructional material of the pharmaceutical kits of the invention may instruct regarding the appropriate or desired use of the included pharmaceutical composition, for example instructing that such pharmaceutical composition (e.g., a PD-1×LAG-3 bispecific molecule) is to be administered for the treatment of cancer. In certain embodiments, the included instructional material of the pharmaceutical kits may instruct that pharmaceutical composition(s) of a PD-1 (or PD-L1)-Binding Molecule, and a LAG-3-Binding Molecule, or a PD-1×LAG-3 (or PD-L1×LAG-3) bispecific molecule of the present invention is administered in combination with a TA-Binding Molecule (optionally having an ADCC-Enhanced Fc Domain) for the treatment of cancer in which a TA (e.g., HER2 or B7-H3) is expressed. Cancers which may be treated include, but are not limited to: adrenal gland cancer, AIDS-associated cancer, alveolar soft part sarcoma, anal cancer (including squamous cell carcinoma of the anal canal (SCAC)), bladder cancer, bone cancer, brain and spinal cord cancer, breast cancer (including, HER2⁺ breast cancer or Triple-Negative Breast Cancer (TNBC)), carotid body tumor, cervical cancer (including, HPV-related cervical cancer), chondrosarcoma, chordoma, chromophobe renal cell carcinoma, clear cell carcinoma, colon cancer, colorectal cancer, desmoplastic small round cell tumor, ependymoma, endometrial cancer (including, unselected endometrial cancer, MSI-high endometrial cancer, dMMR endometrial cancer, and/or POLE exonuclease domain mutation positive endometrial cancer), Ewing's sarcoma, extraskeletal myxoid chondrosarcoma, gallbladder or bile duct cancer (including, cholangiocarcinoma bile duct cancer), gastric cancer, gastroesophageal junction (GEJ) cancer, gestational trophoblastic disease, germ cell tumor, glioblastoma, head and neck cancer (including, squamous cell carcinoma of head and neck (SCCHN)), a hematological malignancy, a hepatocellular carcinoma, islet cell tumor, Kaposi's Sarcoma, kidney cancer, leukemia (including, acute myeloid leukemia), liposarcoma/malignant lipomatous tumor, liver cancer (including, hepatocellular carcinoma liver cancer (HCC)), lymphoma (including, diffuse large B-cell lymphoma (DLBCL), non-Hodgkin's lymphoma (NHL)), lung cancer (including, small cell lung cancer (SCLC), non-small cell lung cancer (NSCLC)), medulloblastoma, melanoma (including, uveal melanoma), meningioma, Merkel cell carcinoma, mesothelioma (including, mesothelial pharyngeal cancer), multiple endocrine neoplasia, multiple myeloma, myelodysplastic syndrome, neuroblastoma, neuroendocrine tumors, ovarian cancer, pancreatic cancer, papillary thyroid carcinoma, parathyroid tumor, pediatric cancer, peripheral nerve sheath tumor, pharyngeal cancer, pheochromocytoma, pituitary tumor, prostate cancer (including, metastatic castration resistant prostate cancer (mCRPC)), posterious uveal melanoma, renal metastatic cancer, rhabdoid tumor, rhabdomyosarcoma, sarcoma, skin cancer, a small round blue cell tumor of childhood (including neuroblastoma and rhabdomyosarcoma), soft-tissue sarcoma, squamous cell cancer, stomach cancer, synovial sarcoma, testicular cancer, thymic carcinoma, thymoma, thyroid cancer, and uterine cancer.

### VII. Uses of the Antibody-Based Molecules of the Invention

As provided herein, the PD-1×LAG-3 bispecific molecules of the present invention can be used to treat or prevent a variety of disorders, including cancer. Additionally, the PD-1-binding (or PD-L1-binding), LAG-3-binding, PD-1×LAG-3 (or PD-L1×LAG-3) bispecific molecules of the present invention can be used in combination with a TA-binding Molecule of the present invention (optionally having an ADCC-Enhanced Fc Domain) to treat a cancer in which such TA is expressed.

Accordingly, the present invention provides methods of treating cancer, such methods comprising administering to a subject in need thereof a PD-1×LAG-3 bispecific molecule.

Additionally, the present invention provides methods of treating cancer comprising administering a TA-Binding Molecule and:
(1) a PD-1×LAG-3 bispecific molecule;
(2) a monospecific PD-1-Binding Molecule, in combination with a monospecific LAG-3-Binding Molecule;
(3) a PD-L1×LAG-3 bispecific molecule; or
(4) a monospecific PD-L1-Binding Molecule, in combination with a monospecific LAG-3-Binding Molecule, wherein such monospecific binding molecule is an intact antibody, and such bispecific molecule is a diabody or a bispecific antibody, and wherein such cancer expresses such TA. In certain embodiments the TA-Binding Molecule comprises an ADCC-Enhanced Fc Domain.

Particular dosing regimens for administering such PD-1×LAG-3 bispecific molecule, or combinations of molecules to a subject in need thereof are provided herein.

As used herein, the term “in combination” refers to the use of more than one therapeutic agent (e.g., an Antibody-Based Molecule of the invention). The use of the term “in combination” does not restrict the order in which individual therapeutic agents are to be administered to a subject with a disease or disorder (e.g., a human patient or other mammal), nor does it mean that the agents are administered or must be administered at exactly the same time, but rather it is meant that such agents are administered to the subject concurrently, or in a sequence within a time interval, such that such agents provide an increased benefit relative to the benefit provided if such agents were administered otherwise. For example, each Antibody-Based Molecule (e.g., a TA-Binding Molecule, a PD-1-Binding Molecule (or a PD-L1-Binding Molecule), and a LAG-3-Binding Molecule; or a TA-Binding Molecule and a PD-1×LAG-3 (or PD-L1×LAG-3) bispecific molecule) may be administered at the same time or sequentially in any order at different points in time; however, if not administered at the same time, they should be administered sufficiently close in time so as to provide the desired therapeutic or prophylactic effect. Each administered agent can be administered separately, in any appropriate form, and by any suitable route, e.g., one by the oral route and one parenterally, etc. Particular dosing regimens for administering the Antibody-Based Molecules of the present invention to a subject in need thereof are provided herein.

The cancers that may be treated by administration of a PD-1×LAG-3 bispecific molecule; or a TA-Binding Molecule and: a PD-1×LAG-3 (or PD-L1×LAG-3) bispecific molecule; or a PD-1-binding (or PD-L1-binding) in combination with a LAG-3-binding Molecule include, but are not limited to: adrenal gland cancer, AIDS-associated cancer, alveolar soft part sarcoma, anal cancer (including squamous cell carcinoma of the anal canal (SCAC)), bladder cancer, bone cancer, brain and spinal cord cancer, breast cancer (including, HER2⁺ breast cancer or Triple-Negative Breast Cancer (TNBC)), carotid body tumor, cervical cancer (including, HPV-related cervical cancer), chondrosarcoma, chordoma, chromophobe renal cell carcinoma, clear cell carcinoma, colon cancer, colorectal cancer, desmoplastic small round cell tumor, ependymoma, endometrial cancer (including, unselected endometrial cancer, MSI-high endometrial cancer, dMMR endometrial cancer, and/or POLE exonuclease domain mutation positive endometrial cancer), Ewing's sarcoma, extraskeletal myxoid chondrosarcoma, gallbladder or bile duct cancer (including, cholangiocarcinoma bile duct cancer), gastric cancer, gastroesophageal junction (GEJ) cancer, gestational trophoblastic disease, germ cell tumor, glioblastoma, head and neck cancer (including, squamous cell carcinoma of head and neck (SCCHN)), a hematological malignancy, a hepatocellular carcinoma, islet cell tumor, Kaposi's Sarcoma, kidney cancer, leukemia (including, acute myeloid leukemia), liposarcoma/malignant lipomatous tumor, liver cancer (including, hepatocellular carcinoma liver cancer (HCC)), lymphoma (including, diffuse large B-cell lymphoma (DLBCL), non-Hodgkin's lymphoma (NHL)), lung cancer (including, small cell lung cancer (SCLC), non-small cell lung cancer (NSCLC)), medulloblastoma, melanoma (including, uveal melanoma), meningioma, Merkel cell carcinoma, mesothelioma (including, mesothelial pharyngeal cancer), multiple endocrine neoplasia, multiple myeloma, myelodysplastic syndrome, neuroblastoma, neuroendocrine tumors, ovarian cancer, pancreatic cancer, papillary thyroid carcinoma, parathyroid tumor, pediatric cancer, peripheral nerve sheath tumor, pharyngeal cancer, pheochromocytoma, pituitary tumor, prostate cancer (including, metastatic castration resistant prostate cancer (mCRPC)), posterious uveal melanoma, renal metastatic cancer, rhabdoid tumor, rhabdomyosarcoma, sarcoma, skin cancer, a small round blue cell tumor of childhood (including neuroblastoma and rhabdomyosarcoma), soft-tissue sarcoma, squamous cell cancer, stomach cancer, synovial sarcoma, testicular cancer, thymic carcinoma, thymoma, thyroid cancer, and uterine cancer.

In certain embodiments, PD-1×LAG-3 bispecific molecules of the present invention may be used in the treatment of: breast cancer (including HER2⁺ breast cancer, and/or TNBC), bile duct cancer (including, cholangiocarcinoma), cervical cancer (including, HPV-related cervical cancer), endometrial cancer (including, unselected endometrial cancer, MSI-high endometrial cancer, dMMR endometrial cancer, and/or POLE exonuclease domain mutation positive endometrial cancer), gastric cancer, GEJ cancer, head and neck cancer (including, SCCHN), liver cancer (including, HCC), lung cancer (including, SCLC and/or NSCLC), lymphoma (including, NHL and DLBCL), ovarian cancer, prostate.

In other embodiments, PD-1-Binding (or PD-L1-Binding) and LAG-3-Binding Molecules, or PD-1×LAG-3 (or PD-L1×LAG-3) bispecific molecules of the present invention may be used in combination with HER2-Binding Molecules (such as margetuximab) in the treatment of HER⁺ cancers, including: breast cancer, metastatic breast cancer, bladder, gastric cancer, GEJ cancer, ovarian cancer, pancreatic cancer, and stomach cancer. In one such embodiment, a PD-1×LAG-3 bispecific molecule is used in combination with ADCC-Enhanced HER2-Binding Molecule. In another of such embodiments, DART-I is used in combination with margetuximab.

In other embodiments, PD-1-binding (or PD-L1-binding) and LAG-3-Binding Molecules, or PD-1×LAG-3 (or PD-L1×LAG-3) bispecific molecules of the present invention may be used in combination with B7-H3-Binding Molecules (such as enoblituzumab) in the treatment of B7-H3⁺ cancers, including: anal cancer, SCAC, a breast cancer, TNBC, a head and neck cancer, SCCHN, lung cancer, NSCLC, melanoma, uveal melanoma, prostate cancer, mCRPC. In one such embodiment, a PD-1×LAG-3 bispecific molecule is used in combination with ADCC-Enhanced B7-H3-Binding Molecule. In another of such embodiments, DART-I is used in combination with enoblituzumab.

In certain embodiments, a PD-1×LAG-3 bispecific molecule; or a TA-Binding Molecule and: a PD-1×LAG-3 (or PD-L1×LAG-3) bispecific molecule; or a PD-1-binding (or PD-L1-binding) in combination with a LAG-3-binding Molecule is/are administered as a first-line therapy for treatment of cancer. In other embodiments, such molecules are administered after one or more prior lines of therapy. In other embodiments, such molecules are administered in further combination with one or more additional therapy. In still other embodiments, such molecules can be employed an adjuvant therapy at the time of, or after surgical removal of a tumor in order to delay, suppress or prevent the development of metastasis. Such molecules can also be administered before surgery (e.g., as a neoadjuvant therapy) in order to decrease the size of the tumor and thus enable or simplify such surgery, spare tissue during such surgery, and/or decrease any resulting disfigurement.

In one embodiment, a PD-1×LAG-3 bispecific molecule is administered in combination with a TA-Binding Molecule (e.g., HER2 or B7-H3) as a first-line therapy for treatment of cancer. In other embodiments, a PD-1×LAG-3 bispecific molecule is administered in combination with a TA-Binding Molecule after one or more prior lines of therapy. In other embodiments, a PD-1×LAG-3 bispecific molecule is administered in combination with a TA-Binding Molecule and in further combination with one or more additional therapy. In still other embodiments, a PD-1×LAG-3 bispecific molecule of the present invention can be employed in combination with a TA-Binding Molecule as an adjuvant therapy at the time of, or after surgical removal of a tumor. A PD-1×LAG-3 bispecific molecule of the present invention can also be administered in combination with a TA-Binding Molecule or a before surgery. In one such embodiment, the TA-Binding Molecule is a HER2-Binding Molecule or a B7-H3-Binding Molecule.

The invention specifically encompasses administering a PD-1×LAG-3 bispecific molecule; or PD-1-Binding (or PD-L1-Binding) and LAG-3-Binding Molecule, or PD-1×LAG-3 (or PD-L1×LAG-3) bispecific molecule in combination with a TA-Binding Molecule in combination with one or more other therapies known to those skilled in the art for the treatment or prevention of cancer, including but not limited to, current standard and experimental chemotherapies, hormonal therapies, biological therapies, immunotherapies, radiation therapies, or surgery. In some embodiments, a combination of a PD-1-binding (or PD-L1-binding) and LAG-3-binding Molecules, or a PD-1×LAG-3 (or PD-L1×LAG-3) bispecific molecule is administered in combination with a TA-Binding Molecule (e.g., an ADCC-Enhanced TA-Binding Molecule) in further combination with a therapeutically or prophylactically effective amount of one or more therapeutic agents known to those skilled in the art for the treatment and/or prevention of cancer, in particular a TA-expressing cancer (e.g., a HER2⁺ cancer or a B7-H3⁺ cancer). Chemotherapeutic agents commonly used in the treatment of HER2 expressing cancers include, but are not limited to anthracyclines (particularly, daunorubicin, doxorubicin, and epirubicin), capecitabine, carboplatin, cyclophosphamide, leucovorin, methotrexate, oxaliplatin, taxanes (particularly, docetaxel and paclitaxel), 5-fluorouracil (5-FU).

Another aspect of the present invention involves improved methods for determining subject amenability to such treatment by measuring the extent of PD-L1 expression in a subject's tumor cells prior to commencing treatment. PD-L1 expression in more than 10% of tumor cells has been established as a clinically relevant cut-off point for treatment with certain PD-1-binding (or PD-L1-binding) Molecules. Methods for measuring the extent of PD-L1 expression are known in the art (de Vicente, J. C. et al. (2018) “PD-L1 Expression in Tumor Cells Is an Independent Unfavorable Prognostic Factor in Oral Squamous Cell Carcinoma,” Cancer Epidemiol. Biomarkers Prev. 28(3):546-554; Davis, A. A. et al. (2019) “The Role Of PD-L1 Expression As A Predictive Biomarker: An Analysis Of All US Food And Drug Administration (FDA) Approvals Of Immune Checkpoint Inhibitors,” J. ImmunoTher. Canc.7:278:1-8; Khozin, S. et al. (2017) “Rates Of PD-LExpression Testing In US Community-Based Oncology Practices (uSCPS) For Patients With Metastatic Non-Small Cell Lung Cancer (mNSCLC) Receiving Nivolumab (N) Or Pembrolizumab (P),” J. Clin. Oncol. 35(15_suppl):11596). For example, such measurement may be accomplished using mouse monoclonal PD-L1 antibody (clone 22C3, 1:200 dilution; PD-L1 IHC 22C3 pharmDx; Dako SK006) by using the Dako EnVision Flex+Visualization System (Dako Autostainer). In such an assay, a formalin-fixed, paraffin-embedded tumor biopsy sample is incubated in the presence of monoclonal mouse anti-PD-L1 antibody (Clone 22C3). PD-L1 protein expression is determined using Tumor Proportion Score (TPS), which is the percentage of viable tumor cells showing partial or complete membrane staining at any intensity or by Combined Positive Score (CPS), which is the number of PD-L1 staining cells (tumor cells, lymphocytes, macrophages) divided by the total number of viable tumor cells, multiplied by one hundred.

A finding that a subject's tumors exhibit PD-L1 expression of less than 1% (as determined using a Combined Positive Score (CPS) or a Tumor Proportion Score (TPS) in an IHC analysis) prior to treatment is indicative of the amenability of the patient to the methods of treatment of the present invention, particularly the methods encompassing administering a PD-1-binding (or PD-L1-binding) and LAG-3-binding Molecule, or PD-1×LAG-3 (or PD-L1×LAG-3) bispecific Molecule in combination with an ADCC-Enhanced TA-Binding Molecule. Such amenability is also heightened in subjects who had previously failed to respond to, or had an inadequate response to at least one prior treatment, including prior treatment with a PD-1-Binding Molecule, or a PD-L1-Binding Molecule in the absence of treatment with an ADCC-Enhanced TA-Binding Molecule. The present invention encompasses methods of treating cancer by administering a TA-Binding Molecule and: a PD-1×LAG-3 (or PD-L1×LAG-3) bispecific molecule; or a PD-1-binding (or PD-L1-binding) in combination with a LAG-3-binding Molecule to a subject, wherein PD-L1 expression on the surface of cells of such cancer, prior to such treatment, is less than 1% as determined using a Combined Positive Score (CPS) or a Tumor Proportion Score (TPS).

VII. Administration and Dosage

An Antibody-Based Molecule of the invention (e.g., a PD-1×LAG-3 bispecific molecule) can be administered to a subject, e.g., a subject in need thereof, for example, a human patient, by a variety of methods. For many applications, the route of administration is one of: intravenous injection or infusion (IV), subcutaneous injection (SC), intraperitoneal injection (IP), or intramuscular injection. It is also possible to use intra-articular delivery. Other modes of parenteral administration can also be used. Examples of such modes include: intraarterial, intrathecal, intracapsular, intraorbital, intracardiac, intradermal, transtracheal, subcuticular, intra-articular, subcapsular, subarachnoid, intraspinal, and epidural and intrasternal injection.

The Antibody-Based Molecule of the invention can be administered as a flat dose or as a weight-based dose (e.g., a mg/kg patient weight dose). The dose can also be selected to reduce or avoid production of antibodies against the administered. Dosage regimens are adjusted to provide the desired response, e.g., a therapeutic response or a combinatorial therapeutic effect. Generally, doses of the Antibody-Based Molecules (and optionally a further agent) can be used in order to provide a subject with the agent in bioavailable quantities. As used herein, the term “dose” refers to a specified amount of medication taken at one time. The term “dosage” refers to the administering of a specific amount, number, and frequency of doses over a specified period of time; the term dosage thus includes chronological features, such as duration and periodicity. With respect to the timing of administration of doses (i.e., dosages), the term “about” is intended to denote a range that is ±3 days of a recited administration.

The term, “flat dose,” as used herein, refers to a dose that is independent of the weight of the patient, and includes physically discrete units of the administered Antibody-Based Molecule (e.g., an antibody that binds a TA, an antibody that binds PD-1, an antibody that binds PD-L1, an antibody that binds LAG-3, a PD-1×LAG-3 (or PD-L1×LAG-3) bispecific molecule) that are suitable for use as a unitary dose for the subjects to be treated; wherein each such unit contains a predetermined quantity of such Antibody-Based Molecule (calculated to produce a desired therapeutic effect) in association with a pharmaceutical carrier, and, optionally, in association with a further agent. Single or multiple flat doses may be given. The term “weight-based dose” as used herein, refers to a discrete amount of a molecule of the invention to be administered per a unit of patient weight, for example milligrams of drug per kilograms of a subject's body weight (mg/kg body weight, abbreviated herein as “mg/kg”). The calculated dose will be administered based on the subject's body weight at baseline. Typically, a significant (≥10%) change in body weight from baseline or established plateau weight will prompt recalculation of dose. Single or multiple doses may be administered in a dosing regimen. Compositions comprising an Antibody-Based Molecule may be administered to a subject in need thereof via infusion.

In some embodiments, Antibody-Based Molecules that bind to a TA (particularly, ADCC-Enhanced TA-Binding Molecules), PD-1, or to PD-L1, and/or to LAG-3, are administered to a subject in need thereof accordingly to approved prescribed dosing regimens, which may incorporate flat doses or weight base doses. Approved prescribed dosing regimens for such molecules have been described (e.g., package inserts for trastuzumab, pertuzumab, pembrolizumab, nivolumab, atezolizumab, durvalumab, tafasitamab etc., are available from the U.S. National Library of Medicine website: dailymed.nlm.nih.gov/dailymed/). In certain embodiments, Antibody-Based Molecules that bind to PD-1, or to PD-L1, and/or to LAG-3, are administered to a subject in need thereof at a flat dose of from about 120 mg to about 800 mg. In certain embodiments, Antibody-Based Molecules that bind to a TA (e.g., Antibody-Based Molecules that bind to HER2 or B7-H3) are administered to a subject in need thereof at a weight-based dose of from about 2 mg/kg to about 18 mg/kg.

In certain embodiments, a PD-1×LAG-3 bispecific molecule (e.g., DART-I) is administered to a subject in need thereof at a flat dose of from about 120 mg to about 800 mg. In certain embodiments, a PD-1×LAG-3 bispecific molecule is administered to a subject in need thereof at a flat dose of about 120 mg, about 300 mg, about 400 mg, about 600 mg, or about 800 mg. In specific embodiments, a PD-1×LAG-3 bispecific molecule is administered to a subject in need thereof at a flat dose of about 400 mg. In another specific embodiment, a PD-1×LAG-3 bispecific molecule is administered to a subject in need thereof at a flat dose of about 600 mg. In another specific embodiment, a PD-1×LAG-3 bispecific molecule is administered to a subject in need thereof at a flat dose of about 800 mg. In certain embodiments, an anti-PD-1 antibody (e.g., retifanlimab) is administered to a subject in need thereof at a flat dose of from about 120 mg to about 750 mg. In certain embodiments, an anti-PD-1 antibody is administered to a subject in need thereof at a flat dose of about 375 mg, about 500 mg, or about 750 mg. In specific embodiments, an anti-PD-1 antibody is administered to a subject in need thereof at a flat dose of about 375 mg. In another specific embodiment, an anti-PD-1 antibody is administered to a subject in need thereof at a flat dose of about 500 mg. In certain embodiments, an anti-LAG-3 antibody (e.g., relatlimab) is administered to a subject in need thereof at a flat dose of from about 80 mg to about 200 mg. In certain embodiments, an anti-LAG-3 antibody is administered to a subject in need thereof at a flat dose of about 80 mg, about 100 mg, or about 160 mg. In specific embodiments, an anti-LAG-3 antibody is administered to a subject in need thereof at a flat dose of about 160 mg. With respect to flat doses or flat dosages, the term “about” is intended to denote a range that is ±10% of a recited dose, such that for example, a dose of about 600 mg will be between 540 mg and 660 mg. With respect to dosages, the term “about” is intended to denote a range that is ±3 days of a recited dose.

In certain embodiments, a HER2- or B7-H3-Binding Molecule (e.g., an anti-HER2 antibody, an anti-B7-H3 antibody) is administered to a subject in need thereof at a weight-based dose of from about 2 mg/kg to about 18 mg/kg. In certain embodiments, a HER2- or B7-H3-Binding Molecule is administered to a subject in need thereof at a dose of about 2 mg/kg, about 4 mg/kg, about 6 mg/kg, about 8 mg/kg, about 10 mg/kg, about 15 mg/kg, or about 18 mg/kg. In specific embodiments, a HER2- or B7-H3-Binding Molecule is administered to a subject in need thereof at a dose of about 15 mg/kg. In other specific embodiments, a first dose of a HER2-Binding Molecule is administered to a subject in need thereof at a dose of about 8 mg/kg, followed by one or more additional doses of such HER2-Binding Molecule at a dose of about 6 mg/kg. In other specific embodiments, a first dose of a HER2-Binding Molecule is administered to a subject in need thereof at a dose of about 4 mg/kg, followed by one or more additional doses of such HER2-Binding Molecule at a dose of about 2 mg/kg. With respect to weight-based doses, the term “about” is intended to denote a range that is 10% of a recited dose, such that for example, a dose of about 15 mg/kg will be between 13.6 mg/kg and 16.5 mg/kg.

In certain embodiments, a HER2-Binding Molecule is administered to a subject in need thereof at a flat dose of from about 420 mg to about 1650 mg. In specific embodiments, a HER2-Binding Molecule is administered to a subject in need thereof at a flat dose of about 420 mg. In another specific embodiment, a HER2-Binding Molecule is administered to a subject in need thereof at a flat dose of about 600 mg In other specific embodiments, a HER2-Binding Molecule is administered to a subject in need thereof at a flat dose of about 840 mg. In another specific embodiment, a HER2-Binding Molecule is administered to a subject in need thereof at a flat dose of about 1650 mg. In other specific embodiments, a first dose of a HER2-Binding Molecule is administered to a subject in need thereof at a flat dose of about 840 mg, followed by one or more additional doses of such HER2-Binding Molecule at a flat dose of about 420 mg.

A dosage of an Antibody-Based Molecule (e.g., a dose of an antibody that binds a TA, an antibody that binds PD-1, an antibody that binds PD-L1, an antibody that binds LAG-3, a PD-1×LAG-3 (or PD-L1×LAG-3) bispecific molecule) can be administered at a periodic intervals over a period of time sufficient to encompass at least 2 doses, at least 4 doses, at least 6 doses, at least 12 doses, or at least 24 doses (a course of treatment). For example, a dosage may be administered e.g., once or twice daily, or about one to four times per week. In certain embodiments, a dosage may be administered, once every week (“Q1W”), once every two weeks (“Q2W”), once every three weeks (“Q3W”), once every four weeks (“Q4W”), etc. Such periodic administration may continue for a period of time e.g., for between about 1 to 52 weeks, or for more than 52 weeks. Such course of treatment may be divided into increments, each referred to herein as a “cycle,” of e.g., between 2 to 24 weeks, between about 3 to 7 weeks, about 4 weeks, or about 6 weeks, or about 8 weeks, or about 12 weeks, or about 24 weeks, during which a set number of doses are administered. The dose and/or the frequency of administration may be the same or different during each cycle. Factors that may influence the dosage and timing required to effectively treat a subject, include, e.g., the severity of the disease or disorder, formulation, route of delivery, previous treatments, the general health and/or age of the subject, and the presence of other diseases in the subject. Moreover, treatment of a subject with a therapeutically effective amount of a compound can include a single treatment or can include a series of treatments.

It is contemplated to provide a subject with multiple doses of an Antibody-Based Molecule (e.g., an antibody that binds a TA, an antibody that binds PD-1, an antibody that binds PD-L1, an antibody that binds LAG-3, a PD-1×LAG-3 (or PD-L1×LAG-3) bispecific molecule). The amount of each Antibody-Based Molecule in each such dose may be the same or may vary from the prior administered dose. Thus, for example, the therapy may comprise the administration of a “first” (or “loading”) dose of such Antibody-Based Molecule followed by a lowered “second” dose of such Antibody-Based Molecule. For example, where the first dose of the Antibody-Based Molecule is approximately 8 mg/kg, the second dose will be less than 8 mg/kg, (e.g., about 6 mg/kg). In some embodiments, the subsequent doses are administered at the same concentration as the second lower dose. In some embodiments, the same dose of the Antibody-Based Molecule is administered over the entire course of treatment. In some embodiments, a TA-Binding Molecule that binds HER2 is administered at a first dose of about 4 mg/kg, about 8 mg/kg, or a first flat dose of about 840 mg, followed by administration of a second lower dose, wherein the second dose is administered about three weeks following the administration of the first dose. In some embodiments, additional subsequent doses of the HER2-Binding Molecule are administered, wherein the subsequent doses are administered about three weeks following the administration of the second dose, or previous subsequent dose.

A “dosing regimen” is a dosage administration in which a patient is administered a predetermined dose (or set of such predetermined doses) at a predetermined frequency (or set of such frequencies) for a predetermined periodicity (or periodicities).

A representative dosing regimen comprises administration of a PD-1×LAG-3 bispecific molecule (e.g., DART-I) at a flat dose of about 120 mg Q2W Another representative dosing regimen comprises administration of a PD-1×LAG-3 bispecific molecule at a flat dose of about 300 mg Q2W. Still another representative dosing regimen comprises administration of a PD-1×LAG-3 bispecific molecule at a flat dose of about 300 mg Q3W. Another representative dosing regimen comprises administration of a PD-1×LAG-3 bispecific molecule at a flat dose of about 400 mg Q2W. Another representative dosing regimen comprises administration a PD-1×LAG-3 bispecific molecule at a flat dose of about 400 mg Q3W. Another representative dosing regimen comprises administration of a PD-1×LAG-3 bispecific molecule at a flat dose of about 600 mg Q2W. Still another representative dosing regimen comprises administration of a PD-1×LAG-3 bispecific molecule at a flat dose of about 600 mg Q3W. Other representative dosing regimens comprise administration of a PD-1×LAG-3 bispecific molecule at a flat dose of about 800 mg Q2W or administration of a PD-1×LAG-3 bispecific molecule at a flat dose of about 800 mg Q3W. As provided herein, such dosing regimens may further comprise the administration of a TA-Binding Molecule. In one embodiment, a PD-1×LAG-3 bispecific molecule is administered according to a dosing regimen provided herein in combination with an approved TA-Binding Molecule (e.g., trastuzumab, pertuzumab, etc.), which is administered accordingly an approved prescribed dosing regimen. In one embodiment, a PD-1×LAG-3 bispecific molecule is administered according to a dosing regimen provided herein in combination with an approved ADCC-Enhanced TA-Binding Molecule (e.g., tafasitamab, etc.), which is administered accordingly an approved prescribed dosing regimen. In certain embodiments of the above dosing regimens, the PD-1×LAG-3 bispecific molecule is DART-I. In one such embodiment, DART-I is administered at a flat dose of about 600 mg Q3W. In another such embodiment, DART-I is administered at a flat dose of about 600 mg Q3W in combination with an approved TA-Binding Molecule (e.g., trastuzumab, pertuzumab, etc.), which is administered according to an approved prescribed dosing regimen. In another such embodiment, DART-I is administered at a flat dose of about 600 mg Q3W in combination with an approved ADCC-Enhanced TA-Binding Molecule (e.g., tafasitamab, etc.), which is administered according to an approved prescribed dosing regimen.

Another representative dosing regimen comprises administration of an anti-PD-1 antibody (e.g., retifanlimab) at a flat dose of about 375 mg Q3W, and an anti-LAG-3 antibody (e.g., relatlimab) at a flat dose of about 160 mg Q4W. Another representative dosing regimen comprises administration of an anti-PD-1 antibody at a flat dose of about 500 mg Q4W and an anti-LAG-3 antibody at a flat dose of about 160 mg Q4W. Still another representative dosing regimen comprises administration of an anti-PD-1 antibody at a flat dose of about 750 mg Q4W, and an anti-LAG-3 antibody at a flat dose of about 160 mg Q4W. As provided herein, such dosing regimens may further comprise the administration of a TA-Binding Molecule. In one embodiment, an anti-PD-1 antibody and an anti-LAG-3 antibody are administered according to a dosing regimen provided herein in combination with an approved TA-Binding Molecule (e.g., trastuzumab, pertuzumab, etc.), which is administered accordingly an approved prescribed dosing regimen. In one embodiment, an anti-PD-1 antibody and an anti-LAG-3 antibody are administered according to a dosing regimen provided herein in combination with an approved ADCC-Enhanced TA-Binding Molecule (e.g., tafasitamab, etc.), which is administered accordingly an approved prescribed dosing regimen. In certain embodiments of the above dosing regimens, the anti-PD-1 antibody is retifanlimab and the anti-LAG-3 antibody is relatlimab. In one such embodiment, retifanlimab is administered at a flat dose of about 375 mg Q3W, relatlimab is administered at a flat dose of about 160 mg Q4W, and an approved TA-Binding Molecule (e.g., trastuzumab, pertuzumab, etc.) is administered according to an approved prescribed dosing regimen. In another such embodiment, retifanlimab is administered at a flat dose of about 500 mg Q4W, relatlimab is administered at a flat dose of about 160 mg Q4W, and an approved TA-Binding Molecule (e.g., trastuzumab, pertuzumab, etc.), is administered according to an approved prescribed dosing regimen. In another such embodiment, retifanlimab is administered at a flat dose of about 375 mg Q3W, relatlimab is administered at a flat dose of about 160 mg Q4W, and an approved ADCC-Enhanced TA-Binding Molecule (e.g., tafasitamab, etc.), is administered according to an approved prescribed dosing regimen. In still another such embodiment, retifanlimab is administered at a flat dose of about 500 mg Q4W, relatlimab is administered at a flat dose of about 160 mg Q4W, and an approved ADCC-Enhanced TA-Binding Molecule (e.g., tafasitamab, etc.), is administered according to an approved prescribed dosing regimen.

In one embodiment, a PD-1×LAG-3 bispecific molecule is administered according to a dosing regimen provided herein in combination with an ADCC-Enhanced TA-Binding Molecule. A representative combination dosing regimen comprises administration of a PD-1×LAG-3 bispecific molecule (e.g., DART-I) at a flat dose of about 120 mg Q2W, and an ADCC-enhanced HER2- or B7-H3-Binding Molecule (e.g., margetuximab or enoblituzumab) at a dose of about 2 mg/kg to about 18 mg/kg, administered Q3W. Another representative combination dosing regimen comprises administration of a PD-1×LAG-3 bispecific molecule (e.g., DART-I) at a flat dose of about 120 mg Q3W, and an ADCC-enhanced HER2- or B7-H3-Binding Molecule (e.g., margetuximab or enoblituzumab) at a dose of about 2 mg/kg to about 18 mg/kg, administered Q3W. Another representative dosing regimen comprises administration of a PD-1×LAG-3 bispecific molecule at a flat dose of about 300 mg Q2W, and an ADCC-enhanced HER2- or B7-H3-Binding Molecule at a dose of about 2 mg/kg to about 18 mg/kg, administered Q3W. Still another representative dosing regimen comprises administration of a PD-1×LAG-3 bispecific molecule at a flat dose of about 300 mg Q3W, and an ADCC-enhanced HER2- or B7-H3-Binding Molecule at a dose of about 2 mg/kg to about 18 mg/kg, administered Q3W. Another representative dosing regimen comprises administration of a PD-1×LAG-3 bispecific molecule at a flat dose of about 400 mg Q2W, and an ADCC-enhanced HER2- or B7-H3-Binding Molecule at a dose of about 2 mg/kg to about 18 mg/kg, administered Q3W. Another representative dosing regimen comprises administration of a PD-1×LAG-3 bispecific molecule 400 mg Q3W, and an ADCC-enhanced HER2- or B7-H3-Binding Molecule at a dose of about 2 mg/kg to about 18 mg/kg, administered Q3W. A specific dosing regimen comprises administration of a PD-1×LAG-3 bispecific molecule at a flat dose of about 600 mg Q2W, and an ADCC-enhanced HER2- or B7-H3-Binding Molecule at a dose of about 2 mg/kg to about 18 mg/kg, administered Q3W. Another specific dosing regimen comprises administration of a PD-1×LAG-3 bispecific molecule at a flat dose of about 600 mg Q3W, and an ADCC-enhanced HER2- or B7-H3-Binding Molecule at a dose of about 2 mg/kg to about 18 mg/kg administered Q3W. Another specific dosing regimen comprises administration of a PD-1×LAG-3 bispecific molecule at a flat dose of about 800 mg Q2W, and an ADCC-enhanced HER2- or B7-H3-Binding Molecule at a dose of about 2 mg/kg to about 18 mg/kg, administered Q3W. Another specific dosing regimen comprises administration of a PD-1×LAG-3 bispecific molecule at a flat dose of about 800 mg Q3W, and an ADCC-enhanced HER2- or B7-H3-Binding Molecule at a dose of about 2 mg/kg to about 18 mg/kg, administered Q3W. In certain embodiments of the above dosing regimens, the PD-1×LAG-3 bispecific molecule is DART-I. In some embodiments of the above dosing regimens, the ADCC-enhanced HER2-Binding Molecule is margetuximab. In some embodiments of the above dosing regimens, the ADCC-enhanced B7-H3-Binding Molecule is enoblituzumab.

Preferably, in the above embodiments, administration occurs at the predetermined frequency or periodicity, or within 1-3 days of such scheduled interval, such that administration occurs 1-3 day before, 1-3 days after, or on the day of a scheduled dose, e.g., once every 3 weeks (3 days). Typically, in the above embodiments, the PD-1×LAG-3 bispecific molecule and the ADCC-enhanced HER2- or B7-H3-Binding Molecule are administered by IV infusion within a 24-hour period. In certain embodiments, the PD-1×LAG-3 bispecific molecule and the ADCC-enhanced HER2- or B7-H3-Binding Molecule are administered by IV infusion according to any of the above dosing regimens for a duration (i.e., course of treatment) of at least 1 month or more, at least 3 months or more, or at least 6 months or more, or at least 12 months or more. A treatment duration of at least 6 months or more, or for at least 12 months or more, or until remission of disease or unmanageable toxicity is observed, is particularly contemplated. In certain embodiments, treatment continues for a period of time after remission of disease.

In certain embodiments, the Antibody-Based Molecules are administered by IV infusion. The Antibody-Based molecules are thus typically diluted (separately or together) into an infusion bag comprising a suitable diluent, e.g., 0.9% sodium chloride. Since infusion or allergic reactions may occur, premedication for the prevention of such infusion reactions is recommended and precautions for anaphylaxis should be observed during the antibody administration. Such IV infusion may be administered to the subject over a period of between 30 minutes and 24 hours. In certain embodiments, the IV infusion is delivered over a period of about 30-240 minutes, about 30-180 minutes, about 30-120 minutes, or about 30-90 minutes, or over a period of about 60-90 minutes, or over a period of about 60-75 minutes, or over a lesser period, if the subject does not exhibit signs or symptoms of an adverse infusion reaction.

Although, as discussed above, various dosing and administration routes may be employed in order to provide Antibody-Based Molecules to recipient subjects in need thereof in accordance with the present invention, certain combinations, dosing and administrative routes are particularly provided for use in such treatment. The use of a PD-1×LAG-3 bispecific diabody of the invention (e.g., DART-I) in combination with an anti-HER2 or anti-B7-H3 antibody (e.g., margetuximab, trastuzumab, pertuzumab, and/or enoblituzumab) in such dosing and administration is particularly described herein.

Accordingly, such dosing regimen comprises administration of a PD-1×LAG-3 bispecific diabody at a flat dose of from about 300 mg to about 800 mg and an anti-HER2 or anti-B7-H3 antibody at a dose of from about 2 mg/kg to about 15 mg/kg, and/or at a flat dose of about 420-840 mg, wherein such molecules are administered Q3W (f 3 days). In certain embodiments, the PD-1×LAG-3 bispecific diabody is administered at a flat dose of about 300 mg, about 400 mg, about 600 mg, or about 800 mg and an anti-HER2 or anti-B7-H3 antibody is administered at a dose of about 2 mg/kg, about 4 mg/kg, about 6 mg/kg, about 8 mg/kg, or about 15 mg/kg. In other embodiments, the PD-1×LAG-3 bispecific diabody is administered at a flat dose of about 300 mg, about 400 mg, about 600 mg, or about 800 mg and an anti-HER2 antibody is administered at a flat does of about 420 mg, or about 840 mg.
(A) In certain embodiments, the PD-1×LAG-3 bispecific diabody is administered at a flat dose of about 300 mg. In such embodiments, if the anti-HER2 or anti-B7-H3 antibody that is to be administered is margetuximab or enoblituzumab, respectively, such margetuximab or enoblituzumab is administered at a dose of about 15 mg/kg body weight. Alternatively, if in such embodiments, the anti-HER2 antibody that is to be administered is trastuzumab, a first dosage of trastuzumab is administered at a dose of about 8 mg/kg, followed by one or more additional dosages of trastuzumab each at a dose of about 6 mg/kg, or a first dosage of trastuzumab is administered at a dose of about 4 mg/kg, followed by one or more additional dosages of trastuzumab each at a dose of about 2 mg/kg. Alternatively, if in such embodiments, the anti-HER2 antibody that is to be administered is pertuzumab, a first dosage of pertuzumab is administered at a dose of about 840 mg, followed by one or more additional dosages of pertuzumab each at a dose of about 420 mg.
(B) In certain embodiments, the PD-1×LAG-3 bispecific diabody is administered at a flat dose of about 400 mg in conjunction with an anti-HER2 or anti-B7-H3 antibody. In such embodiments, if the anti-HER2 or anti-B7-H3 antibody that is to be administered is margetuximab or enoblituzumab, respectively, such margetuximab or enoblituzumab is administered at a dose of about 15 mg/kg body weight. Alternatively, if in such embodiments, the anti-HER2 antibody that is to be administered is trastuzumab, a first dosage of trastuzumab is administered at a dose of about 8 mg/kg, followed by one or more additional dosages of trastuzumab each at a dose of about 6 mg/kg, or a first dosage of trastuzumab is administered at a dose of about 4 mg/kg, followed by one or more additional dosages of trastuzumab each at a dose of about 2 mg/kg. Alternatively, if in such embodiments, the anti-HER2 antibody that is to be administered is pertuzumab, a first dosage of pertuzumab is administered at a dose of about 840 mg, followed by one or more additional dosages of pertuzumab each at a dose of about 420 mg.
(C) In certain embodiments, the PD-1×LAG-3 bispecific diabody is administered at a flat dose of about 600 mg. In such embodiments, if the anti-HER2 or anti-B7-H3 antibody that is to be administered is margetuximab or enoblituzumab, respectively, such margetuximab or enoblituzumab is administered at a dose of about 15 mg/kg body weight. In such embodiments, if the anti-HER2 antibody that is to be administered is trastuzumab, a first dosage of trastuzumab is administered at a dose of about 8 mg/kg, followed by one or more additional dosages of trastuzumab each at a dose of about 6 mg/kg, or a first dosage of trastuzumab is administered at a dose of about 4 mg/kg, followed by one or more additional doses of trastuzumab each at a dose of about 2 mg/kg. Alternatively, if in such embodiments, the anti-HER2 antibody that is to be administered is pertuzumab, a first dosage of pertuzumab is administered at a dose of about 840 mg, followed by one or more additional dosages of pertuzumab each at a dose of about 420 mg.
(D) In certain embodiments, the PD-1×LAG-3 bispecific diabody is administered at a flat dose of about 800 mg. In such embodiments, if the anti-HER2 or anti-B7-H3 antibody that is to be administered is margetuximab or enoblituzumab, respectively, such margetuximab or enoblituzumab is administered at a dose of about 15 mg/kg body weight. Alternatively, if the anti-HER2 antibody that is to be administered is trastuzumab, a first dose of trastuzumab is administered at a dose of about 8 mg/kg, followed by one or more additional dosages of trastuzumab each at a dose of about 6 mg/kg, or a first dose of trastuzumab is administered at a dose of about 4 mg/kg, followed by one or more additional dosages of trastuzumab each at a dose of about 2 mg/kg. Alternatively, if the anti-HER2 antibody that is to be administered is pertuzumab, a first dosage of such pertuzumab is administered at a dose of about 840 mg, followed by one or more additional dosages of pertuzumab each at a dose of about 420 mg.

In any of the above embodiments, the PD-1×LAG-3 bispecific diabody and the anti-HER2 or anti-B7-H3 antibody are administered by IV infusion concurrently, sequentially, in an alternating manner, or at different times, within a 24-hour period. In any of the above embodiments, the PD-1×LAG-3 bispecific diabody is DART-I.

The present invention also provides dosing regimens in which the PD-1×LAG-3 bispecific diabody is administered in combination with two different anti-HER2 antibodies (e.g., trastuzumab and pertuzumab) wherein administration of each molecule is according to any of the above embodiments or is according to an approved prescribed dosing regimen.

### IX. Embodiments of the Invention

Having now generally described the invention, the same will be more readily understood through reference to the following numbered Embodiments (“EA” and “EB”), which are provided by way of illustration and are not intended to be limiting of the present invention unless specified:
EA1. A method of treating a cancer comprising administering a PD-1×LAG-3 bispecific molecule to a subject in need thereof, wherein said method comprises administering said PD-1×LAG-3 bispecific molecule to said subject at a flat dose of from about 120 mg to about 800 mg.
EA2. The method of EA1, wherein said cancer is characterized by the expression of a Tumor Antigen (TA), and wherein said method further comprising administering to said subject a Tumor Antigen (TA) Binding Molecule (TA-Binding Molecule).
EA3. A method of treating a cancer in a subject, wherein said cancer is characterized by the expression of a TA, said method comprising administering a TA-Binding Molecule to said subject and further comprising administering to said subject:
   (a) a bispecific (PD-1×LAG-3 bispecific molecule); or
   (b) a molecule that immunospecifically binds PD-1 (PD-1-Binding Molecule) in combination with a molecule that immunospecifically binds LAG-3 (LAG-3-Binding Molecule); or
   (c) a bispecific molecule that immunospecifically binds both PD-L1 and LAG-3 (PD-L1×LAG-3 bispecific molecule); or
   (d) a molecule that immunospecifically binds PD-L1 (PD-L1-Binding Molecule) in combination with a LAG-3-Binding Molecule.
EA4. The method of any one of EA2-EA3, wherein said TA-Binding Molecule comprises an ADCC-Enhanced Fc Domain.
EA5. The method of any one of EA2-EA4, wherein:
   (a) each molecule is in a separate composition; or
   (b) each molecule is in the same composition; or
   (c) said PD-1-Binding Molecule and said LAG-3-Binding Molecule are in the same composition, and said TA-binding molecule is in a separate composition; or
   (d) said PD-L1-Binding Molecule and said LAG-3-Binding Molecule are in the same composition, and said TA-binding molecule is in a separate composition.
EA6. The method of any one of EA2-EA5, wherein said TA-Binding Molecule is an antibody.
EA7. The method of any one of EA2-EA6, wherein said PD-1-Binding Molecule is an antibody.
EA8. The method of any one of EA2-EA6, wherein said PD-L1-Binding Molecule is an antibody.
EA9. The method of any one of EA2-EA8, wherein said LAG-3-Binding Molecule is an antibody.
EA10. The method of any one of EA3-EA6, wherein said method comprises administering said TA-Binding Molecule and said PD-1×LAG-3 bispecific molecule.
EA1i. The method of any one of EA3-EA9, wherein said method comprises administering said TA-Binding Molecule and said PD-1-Binding Molecule in combination with said LAG-3-Binding Molecule.
EA12. The method of any one of EA3-EA6, wherein said method comprises administering said TA-Binding Molecule and said PD-L1×LAG-3 bispecific molecule.
EA13. The method of any one of EA3-EA9, wherein said method comprises administering said TA-Binding Molecule and said PD-L1-Binding Molecule in combination with said LAG-3-Binding Molecule.
EA14. The method of any one of EA4-EA13, wherein said ADCC-Enhanced Fc Domain comprises:
   (a) an engineered glycoform; and/or
   (b) an amino acid substitution relative to a wild-type Fc Region.
EA15. The method of EA14, wherein said ADCC-Enhanced Fc Domain comprises an engineered glycoform that is a complex N-glycoside-linked sugar chain that does not contain fucose, and/or that comprises a bisecting O-GlcNAc.
EA16. The method of EA14 or EA15, wherein said ADCC-Enhanced Fc Domain comprises one or more amino acid substitutions selected from F243L, R292P, Y300L, V305L, I332E, and P396L.
EA17. The method of any one of EA14-EA16, wherein said ADCC-Enhanced Fc Domain comprises an amino acid substitution is selected from the group consisting of:
   (a) one substitution selected from the group consisting of: F243L, R292P, Y300L, V305I, I332E, and P396L;
   (b) two substitutions selected from the group consisting of:
      (1) F243L and P396L;
      (2) F243L and R292P;
      (3) R292P and V305I; and
      (4) S239D and I332E;
   (c) three substitutions selected from the group consisting of:
      (1) F243L, R292P and Y300L;
      (2) F243L, R292P and V305I;
      (3) F243L, R292P and P396L; and
      (4) R292P, V305I and P396L;
   (d) four substitutions selected from the group consisting of:
      (1) F243L, R292P, Y300L and P396L; and
      (2) F243L, R292P, V305I and P396L; or
   (e) five substitutions selected from the group consisting of:
      (1) F243L, R292P, Y300L, V305I and P396L; and
      (2) L235V, F243L, R292P, Y300L and P396L,

wherein the numbering is that of the EU index as in Kabat.
EA18. The method of any one of EA14-EA16, wherein said ADCC-Enhanced Fc Domain comprises the amino acid substitutions: L235V, F243L, R292P, Y300L and P396L, wherein the numbering is that of the EU index as in Kabat.
EA19. The method of any one of EA14-EA16, wherein said ADCC-Enhanced Enhanced Fc Domain comprises the amino acid substitutions: S239D and I332E, wherein the numbering is that of the EU index as in Kabat.
EA20. The method of any one of EA2-EA19, wherein said TA is selected from Table 6A or Table 6B.
EA21. The method of any one of EA2-EA19, wherein said TA-Binding Molecule comprises the VL and VH Domains of an antibody selected from Table 7.
EA22. The method of any one of EA3-EA7, EA9, EA11 or EA14-EA21, wherein said PD-1-Binding Molecule is an antibody that comprises:
   (a) a PD-1 VL Domain that comprises the amino acid sequence of SEQ ID NO:35, and a PD-1 VH Domain that comprises the amino acid sequence of SEQ ID NO:39;
   (b) a VH and VL Domain of an anti-PD-1 antibody selected from Table 1; or
   (c) a light chain and a heavy chain of an anti-PD-1 antibody selected from Table 1.
EA23. The method of any one of EA3-EA6, EA8-EA9, or EA13-EA21, wherein said PD-L1-Binding Molecule is an antibody that comprises:
   (a) a PD-L1 VL Domain that comprises the amino acid sequence of SEQ ID NO:43, and a PD-L1 VH Domain that comprises the amino acid sequence of SEQ ID NO:47;
   (b) a VH and VL Domain of an anti-PD-L1 antibody selected from Table 2; or
   (c) a light chain and a heavy chain of an anti-PD-L1 antibody selected from Table 2.
EA24. The method of any one of EA3-EA9, EA11 or EA13-EA23, wherein said LAG-3-Binding Molecule is an antibody that comprises:
   (a) a LAG-3 VL Domain that comprises the amino acid sequence of SEQ ID NO:51, and a LAG-3 VH Domain that comprises the amino acid sequence of SEQ ID NO:55;
   (b) a VH and VL Domain of an anti-LAG-3 antibody selected from Table 3; or
   (c) a light chain and heavy chain of an anti-LAG-3 antibody selected from Table 3.
EA25. The method of any one of EA1-EA6, EA10, or EA14-EA21, wherein said PD-1×LAG-3 bispecific molecule comprises:
   (a) a PD-1 VL Domain that comprises the amino acid sequence of SEQ ID NO:35, and a PD-1 VH Domain that comprises the amino acid sequence of SEQ ID NO:39, or a VH and VL Domain of an anti-PD-1 antibody selected from Table 1; and/or
   (b) a LAG-3 VL Domain that comprises the amino acid sequence of SEQ ID NO:51, and a LAG-3 VH Domain that comprises the amino acid sequence of SEQ ID NO:55, or a VH and VL Doman of an anti-LAG-3 antibody selected from Table 3; or
   (c) a bispecific Antibody-Based Molecule selected from Tables 4-5.
EA26. The method of any one of EA1-EA6, EA10, or EA14-EA21, wherein said PD-1×LAG-3 bispecific molecule comprises:
   (a) a PD-1-Binding Domain comprising a Light Chain Variable Domain (VL_{PD-1}) that comprises the CDR_{L}1, CDR_{L}2 and CDR_{L}3 of SEQ ID NO:35, and a Heavy Chain Variable Domain (VH_{PD-}1) that comprises the PD-1-specific CDR_{H}1, CDR_{H}2 and CDR_{H}3 of SEQ ID NO:39; and
   (b) a LAG-3-Binding Domain comprising a Light Chain Variable Domain (VL_{LAG-3}) that comprises the CDR_{L}1, CDR_{L}2 and CDR_{L}3 of SEQ ID NO:51, and a Heavy Chain Variable Domain (VH_{LAG-3}) that comprises the LAG-3-specific CDR_{H}1, CDR_{H}2 and CDR_{H}3 of SEQ ID NO:55.
EA27. The method of any one of EA1-EA6, EA10, EA14-EA21 or EA25-EA26, wherein said PD-1×LAG-3 bispecific molecule comprises:
   (a) two of said PD-1-Binding Domains; and
   (b) two of said LAG-3-Binding Domains.
EA28. The method of any one of EA1-EA6, EA10, EA14-EA21, or EA25-EA27, wherein said PD-1×LAG-3 bispecific molecule comprises the VL Domain of SEQ ID NO:35, and the VH Domain of SEQ ID NO:39.
EA29. The method of any one of EA1-EA6, EA10, EA14-EA21, or EA25-EA28, wherein said PD-1×LAG-3 bispecific molecule comprises the VL Domain of SEQ ID NO:51, and the VH Domain of SEQ ID NO:55.
EA30. The method of any one of EA1-EA6, EA10, EA12, EA14-EA21, or EA25-EA29, wherein said PD-1×LAG-3 bispecific molecule or said PD-L1×LAG-3 bispecific molecule comprises an Fc Region.
EA31. The method of EA30, wherein said Fc Region is of the IgG1, IgG2, IgG3, or IgG4 isotype.
EA32. The method of any one of EA30 or EA31, wherein said PD-1×LAG-3 bispecific molecule or said PD-L1×LAG-3 bispecific molecule further comprises a Hinge Domain.
EA33. The method of EA32, wherein said Fc Region and said Hinge Domain are both of the IgG4 isotype, and wherein said Hinge Domain comprises a stabilizing mutation.
EA34. The method of any one of EA30-EA33, wherein said Fc Region is a variant Fc Region that comprises:
   (a) one or more amino acid modifications that reduces the affinity of the variant Fc Region for an FcγR; and/or
   (b) one or more amino acid modifications that enhances the serum half-life of the variant Fc Region.
EA35. The method of EA34, wherein said modifications that reduce the affinity of the variant Fc Region for an FcγR comprise the substitution of L234A; L235A; or L234A and L235A, wherein said numbering is that of the EU index as in Kabat.
EA36. The method of any one of EA34 or EA35, wherein said modifications that enhances the serum half-life of the variant Fc Region comprise the substitution of M252Y; M252Y and S254T; M252Y and T256E; M252Y, S254T and T256E; or K288D and H435K, wherein said numbering is that of the EU index as in Kabat.
EA37. The method of any one of EA1-EA6, EA10, EA14-EA21, or EA25-EA36, wherein said PD-1×LAG-3 bispecific molecule comprises two polypeptide chains of SEQ ID NO:59 and two polypeptide chains of SEQ ID NO:60.
EA38. The method of any one of EA1-EA6, EA10, EA14-EA21, or EA25-EA37, wherein said PD-1×LAG-3 bispecific molecule or said PD-L1×LAG-3 bispecific molecule is administered at a flat dose of about 120 mg.
EA39. The method of any one of EA1-EA6, EA10, EA14-EA21, or EA25-EA37, wherein said PD-1×LAG-3 bispecific molecule or said PD-L1×LAG-3 bispecific molecule is administered at a flat dose of about 300 mg.
EA40. The method of any one of EA1-EA6, EA10, EA14-EA21, or EA25-EA37, wherein said PD-1×LAG-3 bispecific molecule or said PD-L1×LAG-3 bispecific molecule is administered at a flat dose of about 400 mg.
EA41. The method of any one of EA1-EA6, EA10, EA14-EA21, or EA25-EA37, wherein said PD-1×LAG-3 bispecific molecule or said PD-L1×LAG-3 bispecific molecule is administered at a flat dose of about 600 mg.
EA42. The method of any one of EA1-EA6, EA10, EA14-EA21, or EA25-EA37, wherein said PD-1×LAG-3 bispecific molecule or said PD-L1×LAG-3 bispecific molecule is administered at a flat dose of about 800 mg.
EA43. The method of any one of EA1-EA6, EA10, EA14-EA21, or EA25-EA42, wherein said flat dose is administered once about every 2 weeks.
EA44. The method of any one of EA1-EA6, EA10, EA14-EA21, or EA25-EA42, wherein said flat dose is administered once about every 3 weeks.
EA45. The method of any one of EA1-EA6, EA10, EA14-EA21, EA25-EA37, EA40, or
EA43, wherein said PD-1×LAG-3 bispecific molecule or said PD-L1×LAG-3 bispecific molecule is administered at a flat dose of about 400 mg once about every 2 weeks.
EA46. The method of any one of EA1-EA6, EA10, EA14-EA21, EA25-EA37, EA41, or
EA43, wherein said PD-1×LAG-3 bispecific molecule or said PD-L1×LAG-3 bispecific molecule is administered at a flat dose of about 600 mg once about every 2 weeks.
EA47. The method of any one of EA1-EA6, EA10, EA14-EA21, EA25-EA37, EA41, or
EA44, wherein said PD-1×LAG-3 bispecific molecule or said PD-L1×LAG-3 bispecific molecule is administered at a flat dose of about 600 mg once about every 3 weeks.
EA48. The method of any one of EA1-EA6, EA10, EA14-EA21, EA25-EA37, EA42, or
EA44, wherein said PD-1×LAG-3 bispecific molecule or said PD-L1×LAG-3 bispecific molecule is administered at a flat dose of about 800 mg once about every 3 weeks.
EA49. The method of any one of EA1-EA6, EA10, EA14-EA21, EA25-EA48, wherein said PD-1×LAG-3 bispecific molecule or said PD-L1×LAG-3 bispecific molecule is administered by intravenous (IV) infusion.
EA50. The method of EA49, wherein said intravenous (IV) infusion is over a period of 30-240 minutes.
EA51. The method of EA49, wherein said intravenous (IV) infusion is over a period of about 30-90 minutes.
EA52. The method of any one of EA1-EA51, wherein said cancer is adrenal gland cancer, AIDS-associated cancer, alveolar soft part sarcoma, anal cancer (including squamous cell carcinoma of the anal canal (SCAC)), bladder cancer, bone cancer, brain and spinal cord cancer, breast cancer (including, HER2⁺ breast cancer or Triple-Negative Breast Cancer (TNBC)), carotid body tumor, cervical cancer (including, HPV-related cervical cancer), chondrosarcoma, chordoma, chromophobe renal cell carcinoma, clear cell carcinoma, colon cancer, colorectal cancer, desmoplastic small round cell tumor, ependymoma, endometrial cancer (including, unselected endometrial cancer, MSI-high endometrial cancer, dMMR endometrial cancer, and/or POLE exonuclease domain mutation positive endometrial cancer), Ewing's sarcoma, extraskeletal myxoid chondrosarcoma, gallbladder or bile duct cancer (including, cholangiocarcinoma bile duct cancer), gastric cancer, gastroesophageal junction (GEJ) cancer, gestational trophoblastic disease, germ cell tumor, glioblastoma, head and neck cancer (including, squamous cell carcinoma of head and neck (SCCHN)), a hematological malignancy, a hepatocellular carcinoma, islet cell tumor, Kaposi's Sarcoma, kidney cancer, leukemia (including, acute myeloid leukemia), liposarcoma/malignant lipomatous tumor, liver cancer (including, hepatocellular carcinoma liver cancer (HCC)), lymphoma (including, diffuse large B-cell lymphoma (DLBCL), non-Hodgkin's lymphoma (NHL)), lung cancer (including, small cell lung cancer (SCLC), non-small cell lung cancer (NSCLC)), medulloblastoma, melanoma (including, uveal melanoma), meningioma, Merkel cell carcinoma, mesothelioma (including, mesothelial pharyngeal cancer), multiple endocrine neoplasia, multiple myeloma, myelodysplastic syndrome, neuroblastoma, neuroendocrine tumors, ovarian cancer, pancreatic cancer, papillary thyroid carcinoma, parathyroid tumor, pediatric cancer, peripheral nerve sheath tumor, pharyngeal cancer, pheochromocytoma, pituitary tumor, prostate cancer (including, metastatic castration resistant prostate cancer (mCRPC)), posterious uveal melanoma, renal metastatic cancer, rhabdoid tumor, rhabdomyosarcoma, sarcoma, skin cancer, a small round blue cell tumor of childhood (including neuroblastoma and rhabdomyosarcoma), soft-tissue sarcoma, squamous cell cancer, stomach cancer, synovial sarcoma, testicular cancer, thymic carcinoma, thymoma, thyroid cancer, or uterine cancer..
EA53. The method of EA52, wherein said cancer is anal cancer, breast cancer, bile duct cancer, cervical cancer, colorectal cancer, endometrial cancer, gastric cancer, GEJ cancer, head and neck cancer, liver cancer, lung cancer, lymphoma, melanoma, ovarian cancer or prostate cancer.
EA54. The method of any one of EA52 or EA53, wherein said cancer is HER2⁺ breast cancer or TNBC.
EA55. The method of any one of EA52 or EA53, wherein said cancer is a cholangiocarcinoma bile duct cancer.
EA56. The method of any one of EA52 or EA53, wherein said cancer is an HPV-related cervical cancer.
EA57. The method of any one of EA52 or EA53, wherein said cancer is SCCHN.
EA58. The method of any one of EA52 or EA53, wherein said cancer is HCC.
EA59. The method of any one of EA52 or EA53, wherein said cancer is SCLC or NSCLC.
EA60. The method of any one of EA52 or EA53, wherein said cancer is NHL.
EA61. The method of any one of EA52 or EA53, wherein said cancer is prostate cancer.
EA62. The method of any one of EA52 or EA53, wherein said cancer is gastric cancer.
EA63. The method of any one of EA2-EA62, wherein said TA-Binding Molecule is a HER2-Binding Molecule comprising a HER2-Binding Domain comprising a Light Chain Variable Domain (VL_{HER2}) and a Heavy Chain Variable Domain (VH_{HER2}), wherein:
   (a) said Light Chain Variable Domain (VL_{HER2}) comprises the Light Chain Variable Domain of margetuximab that comprises the CDR_{L}1, CDR_{L}2 and CDR_{L}3 of SEQ ID NO:61, and said Heavy Chain Variable Domain (VH_{HER2}) comprises the Heavy Chain Variable Domain of margetuximab that comprises the CDR_{H}1, CDR_{H}2 and CDR_{H}3 of SEQ ID NO:66;
   (b) said Light Chain Variable Domain (VL_{HER2}) comprises the CDR_{L}1, CDR_{L}2 and CDR_{L}3 of trastuzumab and said Heavy Chain Variable Domain (VH_{HER2}) comprises the CDR_{H}1, CDR_{H}2 and CDR_{H}3 of trastuzumab;
   (c) said Light Chain Variable Domain (VL_{HER2}) comprises the CDR_{L}1, CDR_{L}2 and CDR_{L}3 of pertuzumab and said Heavy Chain Variable Domain (VL_{HER2}) comprises the CDR_{H}1, CDR_{H}2 and CDR_{H}3 of pertuzumab; or
   (d) said Light Chain Variable Domain (VL_{HER2}) comprises the CDR_{L}1, CDR_{L}2 and CDR_{L}3 of hHER2 MAB-1 and said Heavy Chain Variable Domain (VH_{HER2}) comprises the CDR_{H}1, CDR_{H}2 and CDR_{H}3 of hHER2 MAB-1.
EA64. The method of any one of EA2-EA63, wherein said HER2-Binding Molecule is an anti-HER2 antibody.
EA65. The method of EA64, wherein said anti-HER2 antibody is margetuximab, and said method comprises administering margetuximab at a dosage of about 6 mg/kg to about 18 mg/kg once about every 3 weeks.
EA66. The method of EA65, wherein margetuximab is administered once about every 3 weeks at a dose selected from the group consisting of: about 6 mg/kg, about 10 mg/kg, about 15 mg/kg and about 18 mg/kg.
EA67. The method any one of EA65 or EA66, wherein said PD-1×LAG-3 bispecific molecule is administered at a flat dose of about 600 mg once about every 3 weeks and margetuximab is administered at a dose of about 15 mg/kg about once every 3 weeks.
EA68. The method of any one of EA63-EA67, wherein said method further comprises administering a chemotherapeutic agent.
EA69. The method of any one of EA63-EA68, wherein said cancer is a HER2 expressing cancer.
EA70. The method of EA69, wherein said HER2 expressing cancer is breast cancer, metastatic breast cancer, bladder, gastric cancer, GEJ cancer, ovarian cancer, pancreatic cancer, or stomach cancer.
EA71. The method of any one of EA2-EA62, wherein said TA-Binding Molecule is a B7-H3-Binding Molecule comprising a B7-H3-Binding Domain comprising a Light Chain Variable Domain (VL) and a Heavy Chain Variable Domain (VH), wherein: said VL comprises the CDR_{L}1, CDR_{L}2 and CDR_{L}3 of SEQ ID NO:71, and said VH comprises the CDR_{H}1, CDR_{H}2 and CDR_{H}3 of SEQ ID NO:76.
EA72. The method of any one of EA2-EA62 or EA71, wherein said TA-Binding Molecule is enoblituzumab.
EA73. The method of EA72, wherein said enoblituzumab is administered at a dosage of about 6 mg/kg to about 18 mg/kg once about every 3 weeks.
EA74. The method of EA73, wherein enoblituzumab is administered once about every 3 weeks at a dose selected from the group consisting of: about 6 mg/kg, about 10 mg/kg, about 15 mg/kg and about 18 mg/kg.
EA75. The method any one of EA73 or EA74, wherein said PD-1×LAG-3 bispecific molecule is administered at a flat dose of about 600 mg once about every 3 weeks and enoblituzumab is administered at a dose of about 15 mg/kg about once every 3 weeks.
EA76. The method of any one of EA71-EA75, wherein said cancer is a B7-H3 expressing cancer.
EA77. The method of EA76, wherein said B7-H3 expressing cancer is anal cancer, SCAC, a breast cancer, TNBC, a head and neck cancer, SCCHN, lung cancer, NSCLC, melanoma, uveal melanoma, prostate cancer, mCRPC.
EA78. The method of any one of EA2-EA77, wherein said TA-binding molecule is administered by intravenous (IV) infusion.
EA79. The method of EA78, wherein said IV infusion is over a period of about 30-240 minutes.
EA80. The method of EA78, wherein said IV infusion is over a period of about 30-90 minutes.
EA81. The method of any one of EA1-EA6, EA10, EA14-EA21, EA25-EA80, wherein said PD-1×LAG-3 bispecific molecule and said TA-binding molecule are administered concurrently to said subject in separate pharmaceutical compositions, wherein said separate compositions are administered within a 24-hour period.
EA82. The method of any one of EA1-EA6, EA10, EA14-EA21, EA25-EA80, wherein said PD-1×LAG-3 bispecific molecule and said TA-binding molecule are administered sequentially to said subject in separate pharmaceutical compositions, wherein the second administered composition is administered at least 24 hours after the administration of the first administered composition.
EA83. The method of any one of EA1-EA82, wherein said subject has been previously treated with a CAR T-cell therapy.
EA84. The method of any one of EA1-EA6, EA10, EA14-EA21, EA25-EA82, wherein said PD-1×LAG-3 bispecific molecule or said PD-L1×LAG-3 bispecific molecule is administered concurrently with, or following treatment with a CAR T-cell therapy.
EA85. The method of any one of EA1-EA84, wherein cells expressing LAG-3 are present in a biopsy of said cancer prior to said treatment.
EA86. The method of any of EA1-EA85, wherein cells expressing PD-1 are present in a biopsy of said cancer prior to said treatment.
EA87. The method of EA1-EA86, wherein co-expression of PD-1 and LAG-3 in a biopsy of the cancer prior to the treatment is indicative that said patient is a candidate for such methods.
EA88. The method of EA87, wherein expression is gene expression.
EA89. The method of any one of EA1-EA88, wherein PD-L1 expression on the surface of cells of said cancer, prior to said treatment, is less than 1% as determined using a Combined Positive Score (CPS) or a Tumor Proportion Score (TPS).
EA90. The method of any one of EA1-EA89, wherein said subject previously failed to respond to, or had an inadequate response to at least one prior treatment.
EA91. The method of EA90, wherein at least one of said prior treatments was treatment with a PD-1-Binding Molecule or a PD-L1-Binding Molecule.
EB1. A PD-1×LAG-3 bispecific molecule for use to treat cancer in a subject, wherein said PD-1×LAG-3 bispecific molecule is for administration at a flat dose of from about 120 mg to about 800 mg.

EB2. The PD-1×LAG-3 bispecific molecule of EB1, wherein said cancer is characterized by the expression of a TA, and wherein said PD-1×LAG-3 bispecific molecule is used in combination with a TA-Binding Molecule.
EB3. The combination of:

(I) a TA-Binding Molecule; and

(II) (a) a PD-1×LAG-3 bispecific molecule; or
(b) a PD-1-Binding Molecule in combination with a LAG-3-Binding Molecule; or
(c) a PD-L1×LAG-3 bispecific molecule; or
(d) a PD-L1-Binding Molecule in combination with a LAG-3-Binding Molecule,

to treat a cancer characterized by the expression of said TA.
EB4. The PD-1×LAG-3 bispecific molecule of EB2, or the combination of EB3, or the combination of EB7, wherein said TA-Binding Molecule comprises an ADCC-Enhanced Fc Domain.
EB5. The PD-1×LAG-3 bispecific molecule of any one of EB2, or EB4, or the combination of any one of EB2-4, or the combination of any one of EB7-8, wherein:
   (a) each molecule is in a separate composition; or
   (b) each molecule is in the same composition; or
   (c) said PD-1-Binding Molecule and said LAG-3-Binding Molecule are in the same composition, and said TA-binding molecule is in a separate composition; or
   (d) said PD-L1-Binding Molecule and said LAG-3-Binding Molecule are in the same composition, and said TA-binding molecule is in a separate composition.
EB6. The PD-1×LAG-3 bispecific molecule of any one of EB2, or EB4-EB5, or the combination of any one of EB3-5, or the combination of any one of EB7-EB9, wherein said TA-Binding Molecule is an antibody.
EB7. The combination of any one of EB3-EB6, wherein said PD-1-Binding Molecule is an antibody.
EB8. The combination of any one of EB3-EB6, wherein said PD-L1-Binding Molecule is an antibody.
EB9. The combination of any one of EB3-EB8, wherein said LAG-3-Binding Molecule is an antibody.
EB10. The combination of any one of EB3-EB6, wherein said TA-Binding Molecule and said PD-1×LAG-3 bispecific molecule are used.
EB11. The combination of any one of EB3-EB9, wherein said TA-Binding Molecule and said PD-1-Binding Molecule in combination with said LAG-3-Binding Molecule are used.
EB12. The combination any one of EB3-EB6, wherein said TA-Binding Molecule and said PD-L1×LAG-3 bispecific molecule are used.
EB13. The combination of any one of EB3-EB9, wherein said TA-Binding Molecule and said PD-L1-Binding Molecule in combination with said LAG-3-Binding Molecule are used.
EB14. The PD-1×LAG-3 bispecific molecule of any one of EB4-EB6, or the combination of any one of EB4-EB9, wherein said ADCC-Enhanced Fc Domain comprises:

(a) an engineered glycoform; and/or

(b) an amino acid substitution relative to a wild-type Fc Region.
EB15. The PD-1×LAG-3 bispecific molecule of EB14, or the combination of EB14, wherein said ADCC-Enhanced Fc Domain comprises an engineered glycoform that is a complex N-glycoside-linked sugar chain that does not contain fucose, and/or that comprises a bisecting O-GlcNAc.
EB16. The PD-1×LAG-3 bispecific molecule of EB14 or EB15, or the combination of EB14 or EB15, wherein said ADCC-Enhanced Fc Domain comprises one or more amino acid substitutions selected from F243L, R292P, Y300L, V305I, I332E, and P396L.
EB17. The PD-1×LAG-3 bispecific molecule of any one of EB14-EB16, or the combination of any one of EB14-EB16, wherein said ADCC-Enhanced Fc Domain comprises an amino acid substitution is selected from the group consisting of:

(a) one substitution selected from the group consisting of:
F243L, R292P, Y300L, V305I, I332E, and P396L;

(b) two substitutions selected from the group consisting of:
(1) F243L and P396L;
(2) F243L and R292P;
(3) R292P and V305I; and
(4) S239D and I332E;

(c) three substitutions selected from the group consisting of:
(1) F243L, R292P and Y300L;
(2) F243L, R292P and V305I;
(3) F243L, R292P and P396L; and
(4) R292P, V305I and P396L;

(d) four substitutions selected from the group consisting of:
(1) F243L, R292P, Y300L and P396L; and
(2) F243L, R292P, V305I and P396L; or

(e) five substitutions selected from the group consisting of:
(1) F243L, R292P, Y300L, V305I and P396L; and
(2) L235V, F243L, R292P, Y300L and P396L,
   wherein the numbering is that of the EU index as in Kabat.
EB18. The PD-1×LAG-3 bispecific molecule of any one of EB14-EB16, or the combination of any one of EB14-EB16, wherein said ADCC-Enhanced Fc Domain comprises the amino acid substitutions: L235V, F243L, R292P, Y300L and P396L, wherein the numbering is that of the EU index as in Kabat.
EB19. The PD-1×LAG-3 bispecific molecule of any one of EB14-EB16, or the combination of any one of EB14-EB16, wherein said ADCC-Enhanced Enhanced Fc Domain comprises the amino acid substitutions: S239D and I332E, wherein the numbering is that of the EU index as in Kabat.
EB20. The PD-1×LAG-3 bispecific molecule of any one of EB2, EB4-EB6, or EB14-EB19, or the combination of any one of EB3-EB19, wherein said TA is selected from Table 6A or Table 6B.
EB21. The PD-1×LAG-3 bispecific molecule of any one of EB2, EB4-EB6, or EB14-EB19, or the combination of any one of EB3-EB19, wherein said TA-Binding Molecule comprises the VL and VH Domains of an antibody selected from Table 7.
EB22. The combination of any one of EB3-EB7, EB9, EB11 or EB14-EB21, wherein said PD-1-Binding Molecule is an antibody that comprises:
   (a) a PD-1 VL Domain that comprises the amino acid sequence of SEQ ID NO:35, and a PD-1 VH Domain that comprises the amino acid sequence of SEQ ID NO:39;
   (b) a VH and VL Domain of an anti-PD-1 antibody selected from Table 1; or
   (c) a light chain and a heavy chain of an anti-PD-1 antibody selected from Table 1.
EB23. The combination of any one of EB3-EB6, EB8-EB9, or EB13-EB21, wherein said PD-L1-Binding Molecule is an antibody that comprises:
   (a) a PD-L1 VL Domain that comprises the amino acid sequence of SEQ ID NO:43, and a PD-L1 VH Domain that comprises the amino acid sequence of SEQ ID NO:49;
   (b) a VH and VL Domain of an anti-PD-L1 antibody selected from Table 2; or
   (c) a light chain and a heavy chain of an anti-PD-L1 antibody selected from Table 2.
EB24. The combination of any one of EB3-EB9, EB11 or EB13-EB23, wherein said LAG-3-Binding Molecule is an antibody that comprises:
   (a) a LAG-3 VL Domain that comprises the amino acid sequence of SEQ ID NO:51, and a LAG-3 VH Domain that comprises the amino acid sequence of SEQ ID NO:55;
   (b) a VH and VL Domain of an anti-LAG-3 antibody selected from Table 3; or
   (c) a light chain and heavy chain of an anti-LAG-3 antibody selected from Table 3.
EB25. The PD-1×LAG-3 bispecific molecule of any one of EB2, EB4-EB6, or EB14-EB21, or the combination of any one of EB3-EB6, EB10, or EB14-EB21, wherein said PD-1×LAG-3 bispecific molecule comprises:
   (a) a PD-1 VL Domain that comprises the amino acid sequence of SEQ ID NO:35, and a PD-1 VH Domain that comprises the amino acid sequence of SEQ ID NO:39, or a VH and VL Domain of an anti-PD-1 antibody selected from Table 7; and/or
   (b) a LAG-3 VL Domain that comprises the amino acid sequence of SEQ ID NO:51, and a LAG-3 VH Domain that comprises the amino acid sequence of SEQ ID NO:55, or a VH and VL Doman of an anti-LAG-3 antibody selected from Table 9; or
   (c) a bispecific Antibody-Based Molecule selected from Tables 4-5.
EB26. The PD-1×LAG-3 bispecific molecule of any one of EB2, EB4-EB6, or EB14-EB21, or the combination of any one of EB3-EB6, EB10, or EB14-EB21, wherein said PD-1×LAG-3 bispecific molecule comprises:
   (a) a PD-1-Binding Domain comprising a Light Chain Variable Domain (VL_{PD-1}) that comprises the CDR_{L}1, CDR_{L}2 and CDR_{L}3 of SEQ ID NO:35, and a Heavy Chain Variable Domain (VH_{PD-1}) that comprises the PD-1-specific CDR_{H}1, CDR_{H}2 and CDR_{H}3 of SEQ ID NO:39; and
   (b) a LAG-3-Binding Domain comprising a Light Chain Variable Domain (VL_{LAG-3}) that comprises the CDR_{L}1, CDR_{L}2 and CDR_{L}3 of SEQ ID NO:51, and a Heavy Chain Variable Domain (VH_{LAG-3}) that comprises the LAG-3-specific CDR_{H}1, CDR_{H}2 and CDR_{H}3 of SEQ ID NO:55.
EB27. The PD-1×LAG-3 bispecific molecule of any one of EB2, EB4-EB6, EB14-EB21, or EB25-EB26, or the combination of any one of EB3-EB6, EB10, EB14-EB21 or EB25-EB26, wherein said PD-1×LAG-3 bispecific molecule comprises:

(a) two of said PD-1-Binding Domains; and

(b) two of said LAG-3-Binding Domains.
EB28. The PD-1×LAG-3 bispecific molecule of any one of EB2, EB4-EB6, EB14-EB21, or EB25-EB27, or the combination of any one of EB3-EB6, EB10, EB14-EB21, or EB25-EB27, wherein said PD-1×LAG-3 bispecific molecule comprises the VL Domain of SEQ ID NO:35, and the VH Domain of SEQ ID NO:39.
EB29. The PD-1×LAG-3 bispecific molecule of any one of EB2, EB4-EB6, EB14-EB21, or EB25-EB28, or the combination of any one of EB3-EB6, EB10, EB14-EB21, or EB25-EB28, wherein said PD-1×LAG-3 bispecific molecule comprises the VL Domain of SEQ ID NO:51, and the VH Domain of SEQ ID NO:39.
EB30. The PD-1×LAG-3 bispecific molecule of any one of EB2, EB4-EB6, EB14-EB21, or EB25-EB29, or the combination of any one of EB2-6, EB10, 12, EB14-EB21, or EB25-EB29, wherein said PD-1×LAG-3 bispecific molecule or said PD-L1×LAG-3 bispecific molecule comprises an Fc Region.
EB31. The PD-1×LAG-3 bispecific molecule of any one of EB30, or the combination of EB30, wherein said Fc Region is of the IgG1, IgG2, IgG3, or IgG4 isotype.
EB32. The PD-1×LAG-3 bispecific molecule of any one of EB30 or EB31, or the combination of any one of EB30 or EB31, wherein said PD-1×LAG-3 bispecific molecule or said PD-L1×LAG-3 bispecific molecule further comprises a Hinge Domain.
EB33. The PD-1×LAG-3 bispecific molecule of EB32, or the combination of EB32, wherein said Fc Region and said Hinge Domain are both of the IgG4 isotype, and wherein said Hinge Domain comprises a stabilizing mutation.
EB34. The PD-1×LAG-3 bispecific molecule of any one of EB30-EB33, or the combination of any one of EB30-EB33, wherein said Fc Region is a variant Fc Region that comprises:
   (a) one or more amino acid modifications that reduces the affinity of the variant Fc Region for an FcγR; and/or
   (b) one or more amino acid modifications that enhances the serum half-life of the variant Fc Region.
EB35. The PD-1×LAG-3 bispecific molecule of EB34, or the combination of EB34, wherein said modifications that reduce the affinity of the variant Fc Region for an FcγR comprise the substitution of L234A; L235A; or L234A and L235A, wherein said numbering is that of the EU index as in Kabat.
EB36. The PD-1×LAG-3 bispecific molecule of any one of EB34 or EB35, or the combination of any one of EB34 or EB35, wherein said modifications that enhances the serum half-life of the variant Fc Region comprise the substitution of M252Y; M252Y and S254T; M252Y and T256E; M252Y, S254T and T256E; or K288D and H435K, wherein said numbering is that of the EU index as in Kabat. EB37. The PD-1×LAG-3 bispecific molecule of any one of EB2, EB4-EB6, EB14-EB21, or EB25-EB36, or the combination of any one of EB3-EB6, EB10, EB14-EB21, or EB25-EB36, wherein said PD-1×LAG-3 bispecific molecule comprises two polypeptide chains of SEQ ID NO:59 and two polypeptide chains of SEQ ID NO::60.
EB38. The PD-1×LAG-3 bispecific molecule of any one of EB2, EB4-EB6, EB14-EB21, or EB25-EB37, or the combination of any one of EB3-EB6, EB10, EB14-EB21, or EB25-EB37, wherein said PD-1×LAG-3 bispecific molecule or said PD-L1×LAG-3 bispecific molecule is for administration at a flat dose of about 120 mg.
EB39. The PD-1×LAG-3 bispecific molecule of any one of EB2, EB4-EB6, EB14-EB21, or EB25-EB37, or the combination of any one of EB3-EB6, EB10, EB14-EB21, or EB25-EB37, wherein said PD-1×LAG-3 bispecific molecule or said PD-L1×LAG-3 bispecific molecule is for administration at a flat dose of about 300 mg.
EB40. The PD-1×LAG-3 bispecific molecule of any one of EB2, EB4-EB6, EB14-EB21, or EB25-EB37, or the combination of any one of EB3-EB6, EB10, EB14-EB21, or EB25-EB37, wherein said PD-1×LAG-3 bispecific molecule or said PD-L1×LAG-3 bispecific molecule is for administration at a flat dose of about 400 mg.
EB41. The PD-1×LAG-3 bispecific molecule of any one of EB2, EB4-EB6, EB14-EB21, or EB25-EB37, or the combination of any one of EB3-EB6, EB10, EB14-EB21, or EB25-EB37, wherein said PD-1×LAG-3 bispecific molecule or said PD-L1×LAG-3 bispecific molecule is for administration at a flat dose of about 600 mg.
EB42. The PD-1×LAG-3 bispecific molecule of any one of EB2, EB4-EB6, EB14-EB21, or EB25-EB37, or the combination of any one of EB3-EB6, EB10, EB14-EB21, or EB25-EB37, wherein said PD-1×LAG-3 bispecific molecule or said PD-L1×LAG-3 bispecific molecule is for administration at a flat dose of about 800 mg.
EB43. The PD-1×LAG-3 bispecific molecule of any one of EB2, EB4-EB6, EB14-EB21, or EB25-EB42, or the combination of any one of EB3-EB6, EB10, EB14-EB21, or EB25-EB42, wherein said flat dose is for administration once about every 2 weeks.
EB44. The PD-1×LAG-3 bispecific molecule of any one of EB2, EB4-EB6, EB14-EB21, or EB25-EB42, or the combination of any one of EB3-EB6, EB10, EB14-EB21, or EB25-EB42, wherein said flat dose is for administration once about every 3 weeks.
EB45. The PD-1×LAG-3 bispecific molecule of any one of EB2, EB4-EB6, EB14-EB21, EB25-EB37, EB40, or EB43, or the combination of any one of EB3-EB6, EB10, EB14-EB21, EB25-EB37, EB40, or EB43, wherein said PD-1×LAG-3 bispecific molecule or said PD-L1×LAG-3 bispecific molecule is for administration at a flat dose of about 400 mg once about every 2 weeks.
EB46. The PD-1×LAG-3 bispecific molecule of any one of EB2, EB4-EB6, EB14-EB21, EB25-EB37, EB41, or EB43, or the combination of any one of EB3-EB6, EB10, EB14-EB21, EB25-EB37, EB41, or EB43, wherein said PD-1×LAG-3 bispecific molecule or said PD-L1×LAG-3 bispecific molecule is for administration at a flat dose of about 600 mg once about every 2 weeks.
EB47. The PD-1×LAG-3 bispecific molecule of any one of EB2, EB4-EB6, EB14-EB21, EB25-EB37, EB41, or EB44, or the combination of any one of EB3-EB6, EB10, EB14-EB21, EB25-EB37, EB41, or EB44, wherein said PD-1×LAG-3 bispecific molecule or said PD-L1×LAG-3 bispecific molecule is for administration at a flat dose of about 600 mg once about every 3 weeks.
EB48. The PD-1×LAG-3 bispecific molecule of any one of EB2, EB4-EB6, EB14-EB21, EB25-EB37, EB42, or EB44, or the combination of any one of EB3-EB6, EB10, EB14-EB21, EB25-EB37, EB42, or EB44, wherein said PD-1×LAG-3 bispecific molecule or said PD-L1×LAG-3 bispecific molecule is for administration at a flat dose of about 800 mg once about every 3 weeks.
EB49. The PD-1×LAG-3 bispecific molecule of any one of EB2, EB4-EB6, EB14-EB21, or EB25-EB48, or the combination of any one of EB3-EB6, EB10, EB14-EB21, EB25-EB48, wherein said PD-1×LAG-3 bispecific molecule or said PD-L1×LAG-3 bispecific molecule is for administration by intravenous (IV) infusion.
EB50. The PD-1×LAG-3 bispecific molecule of EB49, or the combination of EB49, wherein said intravenous (IV) infusion is over a period of 30-240 minutes.
EB51. The PD-1×LAG-3 bispecific molecule of EB49, or the combination of EB49, wherein said intravenous (IV) infusion is over a period of about 30-90 minutes.
EB52. The PD-1×LAG-3 bispecific molecule of any one of EB2, EB4-EB6, EB14-EB21, or EB25-EB51, or the combination of any one of EB3-EB51, wherein said cancer is adrenal gland cancer, AIDS-associated cancer, alveolar soft part sarcoma, anal cancer (including squamous cell carcinoma of the anal canal (SCAC)), bladder cancer, bone cancer, brain and spinal cord cancer, breast cancer (including, HER2⁺ breast cancer or Triple-Negative Breast Cancer (TNBC)), carotid body tumor, cervical cancer (including, HPV-related cervical cancer), chondrosarcoma, chordoma, chromophobe renal cell carcinoma, clear cell carcinoma, colon cancer, colorectal cancer, desmoplastic small round cell tumor, ependymoma, endometrial cancer (including, unselected endometrial cancer, MSI-high endometrial cancer, dMMR endometrial cancer, and/or POLE exonuclease domain mutation positive endometrial cancer), Ewing's sarcoma, extraskeletal myxoid chondrosarcoma, gallbladder or bile duct cancer (including, cholangiocarcinoma bile duct cancer), gastric cancer, gastroesophageal junction (GEJ) cancer, gestational trophoblastic disease, germ cell tumor, glioblastoma, head and neck cancer (including, squamous cell carcinoma of head and neck (SCCHN)), a hematological malignancy, a hepatocellular carcinoma, islet cell tumor, Kaposi's Sarcoma, kidney cancer, leukemia (including, acute myeloid leukemia), liposarcoma/malignant lipomatous tumor, liver cancer (including, hepatocellular carcinoma liver cancer (HCC)), lymphoma (including, diffuse large B-cell lymphoma (DLBCL), non-Hodgkin's lymphoma (NHL)), lung cancer (including, small cell lung cancer (SCLC), non-small cell lung cancer (NSCLC)), medulloblastoma, melanoma (including, uveal melanoma), meningioma, Merkel cell carcinoma, mesothelioma (including, mesothelial pharyngeal cancer), multiple endocrine neoplasia, multiple myeloma, myelodysplastic syndrome, neuroblastoma, neuroendocrine tumors, ovarian cancer, pancreatic cancer, papillary thyroid carcinoma, parathyroid tumor, pediatric cancer, peripheral nerve sheath tumor, pharyngeal cancer, pheochromocytoma, pituitary tumor, prostate cancer (including, metastatic castration resistant prostate cancer (mCRPC)), posterious uveal melanoma, renal metastatic cancer, rhabdoid tumor, rhabdomyosarcoma, sarcoma, skin cancer, a small round blue cell tumor of childhood (including neuroblastoma and rhabdomyosarcoma), soft-tissue sarcoma, squamous cell cancer, stomach cancer, synovial sarcoma, testicular cancer, thymic carcinoma, thymoma, thyroid cancer, or uterine cancer..
EB53. The PD-1×LAG-3 bispecific molecule of EB52, or the combination of EB52, wherein said cancer is anal cancer, breast cancer, bile duct cancer, cervical cancer, colorectal cancer, endometrial cancer, gastric cancer, GEJ cancer, head and neck cancer, liver cancer, lung cancer, lymphoma, melanoma, ovarian cancer or prostate cancer.
EB54. The PD-1×LAG-3 bispecific molecule of any one of EB52 or EB53, or the combination of any one of EB52 or EB53, wherein said cancer is HER2⁺ breast cancer or TNBC.
EB55. The PD-1×LAG-3 bispecific molecule of any one of EB52 or EB53, or the combination of any one of EB52 or EB53, wherein said cancer is a cholangiocarcinoma bile duct cancer.
EB56. The PD-1×LAG-3 bispecific molecule of any one of EB52 or EB53, or the combination of any one of EB52 or EB53, wherein said cancer is an HPV-related cervical cancer.
EB57. The PD-1×LAG-3 bispecific molecule of any one of EB52 or EB53, or the combination of any one of EB52 or EB53, wherein said cancer is SCCHN.
EB58. The PD-1×LAG-3 bispecific molecule of any one of EB52 or EB53, or the combination of any one of EB52 or EB53, wherein said cancer is HCC.
EB59. The PD-1×LAG-3 bispecific molecule of any one of EB52 or EB53, or the combination of any one of EB52 or EB53, wherein said cancer is SCLC or NSCLC.
EB60. The PD-1×LAG-3 bispecific molecule of any one of EB52 or EB53, or the combination of any one of EB52 or EB53, wherein said cancer is NHL.
EB61. The PD-1×LAG-3 bispecific molecule of any one of EB52 or EB53, or the combination of any one of EB52 or EB53, wherein said cancer is prostate cancer.
EB62. The PD-1×LAG-3 bispecific molecule of any one of EB52 or EB53, or the combination of any one of EB52 or EB53, wherein said cancer is gastric cancer.
EB63. The PD-1×LAG-3 bispecific molecule of any one of EB2, EB4-EB6, EB14-EB21, or EB25-EB62, or the combination of any one of EB3-EB62, wherein said TA-Binding Molecule is a HER2-binding molecule comprising a HER2-Binding Domain comprising a Light Chain Variable Domain (VL_{HER2}) and a Heavy Chain Variable Domain (VH_{HER2}), wherein:
   (a) said Light Chain Variable Domain (VL_{HER2}) comprises the Light Chain Variable Domain of margetuximab that comprises the CDR_{L}1, CDR_{L}2 and CDR_{L}3 of SEQ ID NO:61, and said Heavy Chain Variable Domain (VH_{HER2}) comprises the Heavy Chain Variable Domain of margetuximab that comprises the CDR_{H}1, CDR_{H}2 and CDR_{H}3 of SEQ ID NO:66;
   (b) said Light Chain Variable Domain (VL_{HER2}) comprises the CDR_{L}1, CDR_{L}2 and CDR_{L}3 of trastuzumab and said Heavy Chain Variable Domain (VL_{HER2}) comprises the CDR_{H}1, CDR_{H}2 and CDR_{H}3 of trastuzumab;
   (c) said Light Chain Variable Domain (VL_{HER2}) comprises the CDR_{L}1, CDR_{L}2 and CDR_{L}3 of pertuzumab and said Heavy Chain Variable Domain (VL_{HER2}) comprises the CDR_{H}1, CDR_{H}2 and CDR_{H}3 of pertuzumab; or
   (d) said Light Chain Variable Domain (VL_{HER2}) comprises the CDR_{L}1, CDR_{L}2 and CDR_{L}3 of hHER2 MAB-1 and said Heavy Chain Variable Domain (VH_{HER2}) comprises the CDR_{H}1, CDR_{H}2 and CDR_{H}3 of hHER2 MAB-1.
EB64. The PD-1×LAG-3 bispecific molecule of any one of EB2, EB4-EB6, EB14-EB21, or EB25-EB63, or the combination of any one of EB3-EB63, wherein said HER2-binding molecule is an anti-HER2 antibody.
EB65. The PD-1×LAG-3 bispecific molecule of EB64, or the combination of EB64, wherein said anti-HER2 antibody is margetuximab, and wherein margetuximab is for administration at a dosage of about 6 mg/kg to about 18 mg/kg once about every 3 weeks.
EB66. The PD-1×LAG-3 bispecific molecule of EB65, or the combination of EB65, wherein margetuximab is for administration once about every 3 weeks at a dose selected from the group consisting of about 6 mg/kg, about 10 mg/kg, about 15 mg/kg and about 18 mg/kg.
EB67. The PD-1×LAG-3 bispecific molecule of any one of EB65 or EB66, or the combination any one of EB65 or EB66, wherein said PD-1×LAG-3 bispecific molecule is for administration at a flat dose of about 600 mg once about every 3 weeks and margetuximab is for administration at a dose of about 15 mg/kg about once every 3 weeks.
EB68. The PD-1×LAG-3 bispecific molecule of any one of EB63-67, or the combination of any one of EB63-EB67, wherein said PD-1×LAG-3 bispecific molecule or said combination is for administration with a chemotherapeutic agent.
EB69. The PD-1×LAG-3 bispecific molecule of any one of EB63-EB68, or the combination of any one of EB63-EB68, wherein said cancer is a HER2 expressing cancer.
EB70. The PD-1×LAG-3 bispecific molecule of EB69, or the combination of EB69, wherein said HER2 expressing cancer is breast cancer, metastatic breast cancer, bladder, gastric cancer, GEJ cancer, ovarian cancer, pancreatic cancer, or stomach cancer.
EB71. The PD-1×LAG-3 bispecific molecule of any one of EB2, EB4-EB6, EB14-EB21, or EB25-EB62, or the combination of any one of EB3-EB62, wherein said TA-Binding Molecule is a B7-H3-binding molecule comprising a B7-H3-Binding Domain comprising a Light Chain Variable Domain (VL) and a Heavy Chain Variable Domain (VH), wherein:
   said VL comprises the CDR_{L}1, CDR_{L}2 and CDR_{L}3 of SEQ ID NO:71, and said VH comprises the CDR_{H}1, CDR_{H}2 and CDR_{H}3 of SEQ ID NO:76.
EB72. The PD-1×LAG-3 bispecific molecule of any one of EB2, EB4-EB6, EB14-EB21, EB25-EB62, or EB71, or the combination of any one of EB3-62 or EB71, wherein said TA-Binding Molecule is enoblituzumab.
EB73. The PD-1×LAG-3 bispecific molecule of EB72, or the combination of EB72, wherein said enoblituzumab is for administration at a dosage of about 6 mg/kg to about 18 mg/kg once about every 3 weeks.
EB74. The PD-1×LAG-3 bispecific molecule of EB73, or the combination of EB73, wherein enoblituzumab is for administration once about every 3 weeks at a dose selected from the group consisting of about 6 mg/kg, about 10 mg/kg, about 15 mg/kg and about 18 mg/kg.
EB75. The PD-1×LAG-3 bispecific molecule of any one of EB73 or EB74, or the combination any one of EB73 or EB74, wherein said PD-1×LAG-3 bispecific molecule is for administration at a flat dose of about 600 mg once about every 3 weeks and enoblituzumab is for administration at a dose of about 15 mg/kg about once every 3 weeks.
EB76. The PD-1×LAG-3 bispecific molecule of any one of EB71-EB75, or the combination any one of EB71-EB75, wherein said cancer is a B7-H3 expressing cancer.
EB77. The PD-1×LAG-3 bispecific molecule of EB76, or the combination of EB76, wherein said B7-H3 expressing cancer is anal cancer, SCAC, a breast cancer, TNBC, a head and neck cancer, SCCHN, lung cancer, NSCLC, melanoma, uveal melanoma, prostate cancer, mCRPC.
EB78. The PD-1×LAG-3 bispecific molecule of any one of EB2, EB4-EB6, EB14-EB21, or EB25-EB77, or the combination of any one of EB3-EB77, wherein said TA-binding molecule is for administration by intravenous (IV) infusion.
EB79. The PD-1×LAG-3 bispecific molecule of EB78, or the combination of EB78, wherein said IV infusion is over a period of about 30-240 minutes.
EB80. The PD-1×LAG-3 bispecific molecule of EB78, or the combination of EB78, wherein said IV infusion is over a period of about 30-90 minutes.
EB81. The PD-1×LAG-3 bispecific molecule of any one of EB2, EB4-EB6, EB14-EB21, or EB25-EB80, or the combination of any one of EB3-EB6, EB10, EB14-EB21, EB25-EB80, wherein said PD-1×LAG-3 bispecific molecule and said TA-binding molecule are for concurrent administration to said subject in separate pharmaceutical compositions, wherein said separate compositions are for administration within a 24-hour period.
EB82. The PD-1×LAG-3 bispecific molecule of any one of EB2, EB4-EB6, EB14-EB21, or EB25-EB80, or the combination of any one of EB3-EB6, EB10, EB14-EB21, EB25-EB80, wherein said PD-1×LAG-3 bispecific molecule and said TA-binding molecule are for sequential administration to said subject in separate pharmaceutical compositions, wherein the second administered composition for administration at least 24 hours after the administration of the first administered composition.
EB83. The PD-1×LAG-3 bispecific molecule of any one of EB2, EB4-EB6, EB14-EB21, or EB25-EB82, or the combination of any one of EB3-EB82, wherein said subject has been previously treated with a CAR T-cell therapy.
EB84. The PD-1×LAG-3 bispecific molecule of any one of EB2, EB4-EB6, EB14-EB21, or EB25-EB83, or the combination of any one of EB3-EB6, EB10, EB14-EB21,
EB25-EB82, wherein said PD-1×LAG-3 bispecific molecule or said PD-L1×LAG-3 bispecific molecule is for administration concurrently with, or following treatment with a CAR T-cell therapy.
EB85. The PD-1×LAG-3 bispecific molecule of any one of EB2, EB4-EB6, EB14-EB21, or EB25-EB84, or the combination of any one of EB3-EB84, wherein cells expressing LAG-3 are present in a biopsy of said cancer prior to said treatment.
EB86. The PD-1×LAG-3 bispecific molecule of any one of EB2, EB4-EB6, EB14-EB21, or EB25-EB85, or the combination of any of EB3-EB85, wherein cells expressing PD-1 are present in a biopsy of said cancer prior to said treatment.
EB87. The PD-1×LAG-3 bispecific molecule of EB1-EB86, or the combination of any of EB3-EB86, wherein co-expression of PD-1 and LAG-3 in a biopsy of the cancer prior to the treatment is indicative that said patient is a candidate for such methods.
EB88. The PD-1×LAG-3 bispecific molecule of EB87, or the combination of EB87 wherein expression is gene expression.
EB89. The PD-1×LAG-3 bispecific molecule of any one of EB2, EB4-EB6, EB14-EB21, or EB25-EB88, or the combination of any one of EB3-EB88, wherein PD-L1 expression on the surface of cells of said cancer, prior to said treatment, is less than 1% as determined using a Combined Positive Score (CPS) or a Tumor Proportion Score (TPS).
EB90. The PD-1×LAG-3 bispecific molecule of any one of EB2, EB4-EB6, EB14-EB21, or EB25-EB89, or the combination of any one of EB3-EB89, wherein said subject previously failed to respond to, or had an inadequate response to at least one prior treatment.
EB91. The PD-1×LAG-3 bispecific molecule of EB90, or the combination of EB88, wherein at least one of said prior treatments was treatment with a PD-1-Binding Molecule or a PD-L1-Binding Molecule.

### EXAMPLES

The following examples are provided to better illustrate the claimed invention and are not to be interpreted as limiting the scope of the invention. To the extent that specific materials are mentioned, it is merely for purposes of illustration and is not intended to limit the invention. One skilled in the art can develop equivalent means or reactants without the exercise of inventive capacity and without departing from the scope of the invention.

### Example 1

### Phase I Studies

In order to determine the tolerability of patients to DART-I (a bispecific molecule that binds PD-1 and LAG-3 also known as MGD013 and tebotelimab), a Phase I clinical study is being conducted. The study includes a dose escalation phase and a cohort expansion phase. The study was approved by the institutional review boards of each clinical site, and all patients signed a written-informed consent.

For the initial dose escalation and dose expansion cohorts, DART-I was administered once every two weeks (Q2W). For purposes of the study, an eight (8) week (56 day) cycle is used in which DART-I is administered Q2W starting on day 1 of every two week period (i.e., administered on day 1, and on days 15±1 day, 29±1 day, and 43±1 day) of the first cycle, and Q2W starting on day 1±1 day of each subsequent cycle. Patients may receive multiple 8-week Q2W treatment cycles depending on tolerability and response to study treatments.

In additional expansion cohorts, DART-I is administered once every three weeks (Q3W). For purposes of the study, three (3) week cycles (each being of 21 days) are used. DART-I is administered on day 1 of the first cycle and day 1±3 day of each subsequent cycle. Patients may receive multiple 3-week (Q3W) treatment cycles depending on tolerability and response to study treatments.

In combination expansion cohorts, DART-I and an anti-HER2 antibody margetuximab (a TA-Binding Molecule having an ADCC-Enhanced Fc Domain) are both administered once every three weeks (Q3W). For purposes of the study, three (3) week cycles (each being 21 days) are used in which DART-I and margetuximab are administered on day 1 of the first cycle and day 1±3 day of each subsequent cycle. Patients may receive multiple 3-week Q3W treatment cycles depending on tolerability and response to study treatments.

In these studies, doses of DART-I are diluted to a concentration range of 0.12 mg/mL to 6.4 mg/mL in normal saline and administered over about 60 to 75 minutes through an IV line using a commercially available syringe or infusion pump.

In these studies doses of margetuximab are diluted to a concentration of 2.4 to 7.2 mg/mL in normal saline and administered by IV infusion over approximately 30-120 minutes using a commercially available syringe or infusion pump.

Antitumor activity is evaluated using: conventional Response Evaluation Criteria in Solid Tumors (RECIST), version 1.1 (Eisenhauer, E. A., et al. (2009) “New Response Evaluation Criteria In Solid Tumours: Revised RECIST Guideline (Version 1.1)” Eur. J. Cancer. 45(2):228-247); immune-related Response Evaluation Criteria in Solid Tumors (irRECIST) (Wolchok, J. D., et al., (2009) “Guidelines For The Evaluation Of Immune Therapy Activity In Solid Tumors: Immune-Related Response Criteria.” Clin. Cancer Res, 15:7412-7420); or the Revised International Working Group criteria (i.e., the Lugano Classification; Cheson, B. D., et al., (2014) “Recommendations For Initial Evaluation, Staging, And Response Assessment Of Hodgkin And Non-Hodgkin Lymphoma: The Lugano Classification.” J. Clin. Oncol, 32:3059-3068) for response assessment, as applicable.

In the dose escalation phase sequential escalating flat doses from 1 mg to up to 1200 mg were administered Q2W in successive cohorts of 1 to 6 patients, each was evaluated (Table 8). At various dose levels, patients assessed to not be evaluable for dose escalation purposes were replaced. Additional patients were also enrolled at multiple dose levels of interest to gain additional clinical experience. In the dose escalation phase patients with unresectable, locally advanced or metastatic solid tumors of any histology were enrolled. 47 patients (49% checkpoint-experienced) were treated in dose escalation and no maximum tolerated dose was defined.

TABLE 8
Dose Escalation Cohorts

| Dose Level | DART-I Dose | | Cohorts |
|---|---|---|---|
| Dose Level 1 | 1 | mg | Cohort 1 |
| Dose Level 2 | 3 | mg | Cohort 2 |
| Dose Level 3 | 10 | mg | Cohort 3 |
| Dose Level 4 | 30 | mg | Cohort 4 |
| Dose Level 5 | 120 | mg | Cohort 5 |
| Dose Level 6 | 400 | mg | Cohort 6 |
| Dose Level 7 | 800 | mg | Cohort 7 |
| Dose Level 8 | 1200 | mg | Cohort 8 |

Based on the totality of the clinical data from the Dose Escalation Phase, including but not limited to, observed clinical activity, peripheral receptor occupancy, and pharmacokinetics (PK), a dose of 600 mg, administered Q2W, was initially selected as the dosing regimen to be evaluated in a Cohort Expansion Phase.

Patients with distinct malignant diseases (including: NSCLC (post prior checkpoint treatment and checkpoint-naïve cohorts); SCCHN (post prior checkpoint treatment and checkpoint-naïve cohorts); SCLC; cholangiocarcinoma; HCC; cervical cancer; TNBC; epithelial ovarian cancer (EOC); DLBCL, and gastric cancer) are treated with DART-I at a flat dose of 600 mg administered Q2W in the initial cohorts of the Cohort Expansion Phase. Based in part on the pharmacokinetics (PK) and receptor occupancy (RO) data detailed below, additional cohorts of the Cohort Expansion Phase (initially patients with gastric cancer, or EOC) are treated with DART-I at a flat dose of 600 mg administered Q3W.

In a separate cohort enrolling patients with advanced or metastatic solid tumors expressing the HER2 tumor antigen (i.e., HER2+ solid tumors, particularly HER2+ gastric or breast cancer) receive DART-I and margetuximab, administered sequentially on the same day. DART-I (300 mg or 600 mg) is administered followed by administration of margetuximab (15 mg/kg) Q3W. This cohort followed a conventional 3+3 approach beginning with enrollment of 3 patients at the DART-I 300 mg dose level followed by patients treated with DART-I at the 600 mg dose level.

### Pharmacokinetics (PK)

In the ongoing study, the pharmacokinetic profile of DART-I at a dosing regimen range of 1 to 1200 mg Q2W was evaluated. Serum PK samples were collected (i) pre-dose, (ii) at the end of infusion EOI), and (iii) 2, 4, 24, 72, 168 hours after the start of infusion for the first dose Day 1 of Cycles 1-2. Additional serum PK samples were collected pre-dose and EOI for each additional dose administered during Cycles 1-2, and concentrations of DART-I in human serum were measured using ELISA. Briefly, the assay plates were coated overnight with 2 μg/mL of capture antibody (anti-idiotype antibody recognizing the LAG-3 domain of DART-I, “anti-ID”). After blocking the non-specific sites with 0.5% bovine serum albumin (BSA) in 1× phosphate buffered saline (PBS) with 0.1% Tween-20, the plate is incubated with DART-I standard calibrators, quality controls and test samples. The immobilized anti-ID antibody captures the DART-I present in the standard calibrators, quality controls and test samples. The captured DART-I is detected by the sequential addition of 0.25 μg/mL 2A5-Biotin (biotinylated anti-EK coil antibody) followed by 1:10,000 dilution of Streptavidin-HRP. The bound HRP activity is quantified by the luminescence light generation by ELISA PICO substrate. The luminescence light intensity is measured as the relative light unit (RLU) using a Victor X4 plate reader. A standard curve is generated by fitting the RLU signal from DART-I standards with a four-parameter logistic model. The concentration of DART-I in the serum samples is determined by interpolation from a standard curve using a four-parameter curve fit with 1/y² weighting relating the light intensity to the concentration of DART-I.

A preliminary PK compartmental modeling approach was used to analyze the data using the WinNonlin PK analysis program (Phoenix® 64 WinNonlin®, Version 8.0, Certara Inc., Princeton, N.J.). The model used was an open one- or two-compartment and a weighting factor of reciprocal of predicted concentration squared. The model was fitted to the Cycle 1, Day 1 (C1D1), first dose data with WinNonlin generated initial estimates.

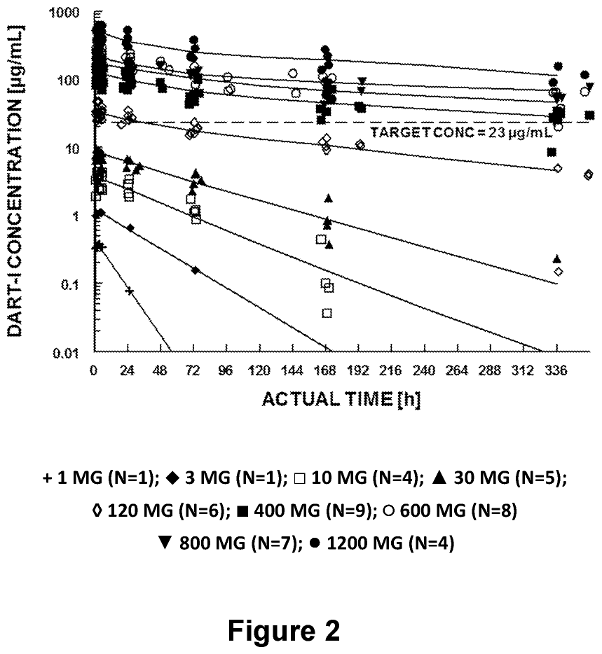

Forty-five subjects (all dosed Q2W) were evaluable for preliminary PK analysis (1 patient each at 1 and 3 mg Q2W dosages, 4 patients at a 10 mg Q2W dosage, 5 patients at a 30 mg Q2W dosage, 6 patients at a 120 mg Q2W dosage, 9 patients at a 400 mg Q2W dosage, 8 patients at a 600 mg Q2W dosage, 7 patients at an 800 mg Q2W dosage, and 4 patients at a 1200 mg Q2W dosage). PK profiles are presented in FIG. 2.

PK parameters are summarized by treatment in Table 9. These results indicated that first dose DART-I exposure increased in a dose-related manner. DART-I Cₘₐₓ increased in a dose proportional manner (slope: 0.985 [90% confidence interval (CI): 0.949 - 1.022]) and first dose AUC_{(INF)} increased in a greater than dose proportional manner (slope: 1.345 [90% CI: 1.294 - 1.397]) over the dose range of 1 to 1200 mg. Total body clearance (CL) values decreased with increasing dose, and both steady state volumes of distribution (Vₛₛ) and elimination half-life (tin) values increased with increasing dose over the dose range of 1 to 1200 mg. However, CL, Vu, and tin appeared to be independent of dose over the dose range of 400 to 1200 mg, although slight trends were noticed with increasing dose. The mean half-life of DART-I was approximately 11 days, and the volume of distribution indicates that DART-I distribution is confined to the blood volume.

TABLE 9
PK Parameters

| | Cₘₐₓ | | AUC_{(INF)} | | CL | | Vₛₛ | | t_{1/2} | |
|---|---|---|---|---|---|---|---|---|---|---|
| | (μg/mL) | | (μ · h/mL) | | (mL/h) | | (mL) | | (h) | |
| DART-I Dosage | GeoMean | | GeoMean | | Mean | | Mean | | Mean | |
| (Dose in mg) | (% CV) | | (% CV) | | (SD) | | (SD) | | (SD) | |
| 1 Q2W (n = 1) | 0.4 | | 6 | | 159.4 | | 2358 | | 10.3 | |
| 3 Q2W (n = 1) | 1.2 | | 44 | | 68.5 | | 2470 | | 25.0 | |
| 10 Q2W (n = 4) | 3.0 | (25) | 207 | (30) | 49.9 | (13.7) | 2818 | (700) | 40.4 | (10.1) |
| 30 Q2W (n = 5) | 8.0 | (13) | 590 | (26) | 52.2 | (12.8) | 3756 | (505) | 51.5 | (9.8) |
| 120 Q2W (n = 6) | 32.9 | (20) | 5503 | (8) | 21.9 | (1.7) | 4442 | (975) | 152.5 | (40.9) |
| 400 Q2W (n = 9) | 119.7 | (26) | 26213 | (41) | 16.5 | (7.8) | 4857 | (1866) | 247.3 | (157.2) |
| 600 Q2W (n = 8) | 198.7 | (26) | 50878 | (35) | 12.5 | (5.4) | 4515 | (1745) | 285.3 | (116.0) |
| 800 Q2W (n = 7) | 201.5 | (20) | 47393 | (50) | 18.5 | (8.2) | 6149 | (1854) | 285.9 | (169.3) |
| 1200 Q2W (n = 4) | 500.0 | (18) | 121384 | (23) | 10.1 | (2.3) | 3888 | (1406) | 288.3 | (106.3) |

| | | | | | | | | |
|---|---|---|---|---|---|---|---|---|
| 400 to 1200 Q2W | NR | NR | 14.9 | (7.1) | 4944 | (1845) | 273.7 | (137.0) |
| Overall (n = 28) | | | | | | | | |

Abbreviations: AUC(INF) = area under the serum concentration time curve from time zero extrapolated to infinite time; C1D1 = Cycle 1 Day 1; Cmax = maximum observed serum concentration; CL = total body clearance; CV = coefficient of variation; GeoMean = geometric mean; N = number of patients; NR = not reported; Q2W = once every 2 weeks; SD = standard deviation; t½ = elimination half-life; Vss = volume of distribution at steady-state.

### Pharmacodynamics (PD)

The receptor occupancy (RO) profile of DART-I in the dose range of 1 to 1200 mg Q2W was evaluated. The RO of DART-I in each sample was determined by fluorescence-activated cell sorting (FACS). Briefly, five aliquots of whole blood per sample are distributed to five 12×75 mm tubes. Two of these aliquots are spiked with DART-I—one spiked sample is meant for each RO panel. After a 30 minute incubation at room temperature (RT), all aliquots are treated with red blood cell lysis buffer (BD Biosciences) at RT in the dark for 15 minutes and then centrifuged at 1200 rpm for 5 minutes. Supernatant is removed and leukocyte containing cell pellet is washed with 2 ml FBS staining buffer (BD Biosciences). Two aliquots (one spiked) are stained with Antibody Panel 1, two aliquots (one spiked) are stained with Antibody Panel 2 (see Table 10), and one aliquot is stained with the appropriate isotype control in a total volume of 100 μL for 30 minutes at RT in the dark. Samples are wash two times with 2 mL FACS buffer. 0.2 μg of Streptavidin, R-Phycoerythrin Conjugate (SAPE, Life Technologies) is added to the Antibody Panel 2 aliquots, which are then mixed and incubated for 30 minutes at RT in the dark, then washed one time with 2 mL FACS buffer. Cells are resuspended in 200 uL of staining buffer with DAPI (0.1 μg/mL) (Panel 1 and 2 samples) or without DAPI (isotype sample) and after 10 minutes are acquired on a FACS Canto II. Geometric Mean Fluorescent Intensity (gMFI) is recorded for the entirety of either the CD4+ or CD8+ population for either the IgG4 or EK channels for all timepoints. The Cycle 1 Day 1 (C1D1) pre-dose sample is considered background to be subtracted from all samples (the isotype sample is used if C1D1 pre-dose sample data is not available). Receptor Occupancy(RO), expressed as percent (%), is calculated using the following formula:

$$Receptor {Occupancy ( RO )}_{EK or IgG 4} = \frac{gMFI {Sample}_{No Spike} - gMFI Background}{gMFI {Sample}_{Spike} - gMFI Background}$$

$$Receptor {Occupancy ( RO )}_{EK or IgG 4} = \frac{gMFI {Sample}_{No Spike} - gMFI Background}{gMFI {Sample}_{Spike} - gMFI Background}$$

TABLE 10
Antibody Panels

| | Panel 1 | Panel 2 |
|---|---|---|
| Fluorescent labeled antibody/source | μL/test | μL/test |
| Alexafluor 488 (AF488)-conjugated anti-PD-1 | 5 | — |
| [non-competing]/MacroGenics | | |
| phycoerythrin (PE)-Cy7 conjugated anti-LAG-3 | — | 5 |
| [clone 3DS223H] (non-competing)/Ebioscience | | |
| Biotin conjugated anti-EK [clone 2A5]/ | — | 10 |
| MacroGenics | | |
| PE conjugated anti-IgG4 [clone HP6023]/Southern | 2 | — |
| Biotech | | |
| PerCP Cy5.5 conjugated anti-CD8 [clone RPA- | 5 | 5 |
| T8]/BD Biosciences | | |
| PE-Cy7 conjugated anti-CD45RA [clone L48]/BD | 5 | 5 |
| Biosciences | | |
| APC conjugated anti-CCR7 [clone G043H7]/ | 5 | 5 |
| BioLegand | | |
| APC-Cy7 conjugated anti-CD4 [clone SK3]/ | 5 | 5 |
| BD Biosciences | | |
| V500 conjugated anti-CD3 [clone SP34-2]/ | 5 | 5 |
| BD Biosciences | | |
| Staining buffer | 68 | 60 |

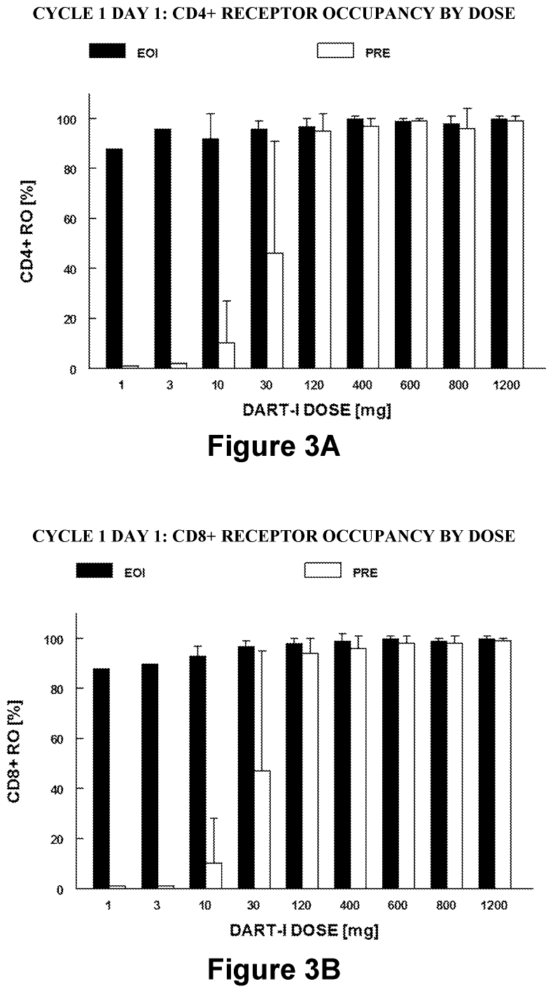

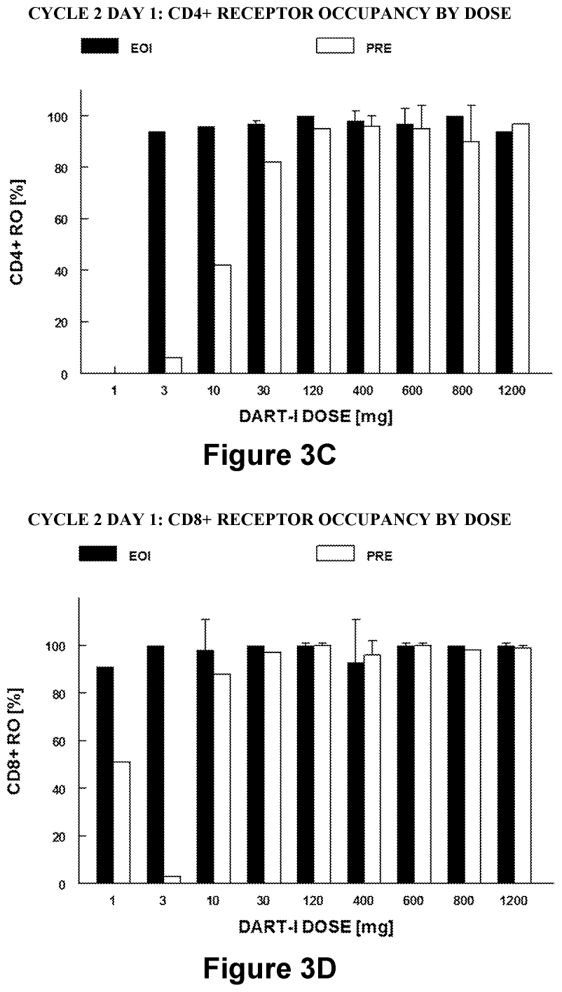

Fifty-six patients (all dosed Q2W) were evaluable for preliminary PD analysis (1 patient each in the 1 and 3 mg Q2W dose, 3 patients in the 10 mg Q2W dose, 5 patients in the 30 mg Q2W dose, 7 patients in the 120 mg Q2W dose, 9 patients in the 400 mg Q2W dose, 16 patients in the 600 mg Q2W dose, 8 patients in the 800 mg Q2W dose, and 6 patients in the 1200 mg Q2W dose). Percent Receptor occupancy (RO) of CD4+ and CD8+ cells at EOI (end of infusion after the administration of the first dose of Cycle 1 or Cycle 2), and PRE (prior to the administration of the next dose) are presented in FIGS. 3A-3D. The relationship between DART-I concentration and binding to CD4+ and CD8+ cell was examined using an Emax model: E=(Emax*C)/(EC50+C); where E=% binding, Emax=maximal % binding, EC50=conc producing half maximal effect, and C=conc of DART-I. DART-I was found to demonstrate potent RO with and EC50 of 0.045 and 0.011 μg/mL for CD4+ and CD8+ cells respectively. Maximum RO was observed at doses ≥120 mg over the entire Q2W dosing interval, and 90% of max RO is achieved at 0.6 and 0.1 μg/mL for CD4+ and CD8+ cells, respectively.

### PK and Target Concentration Modeling

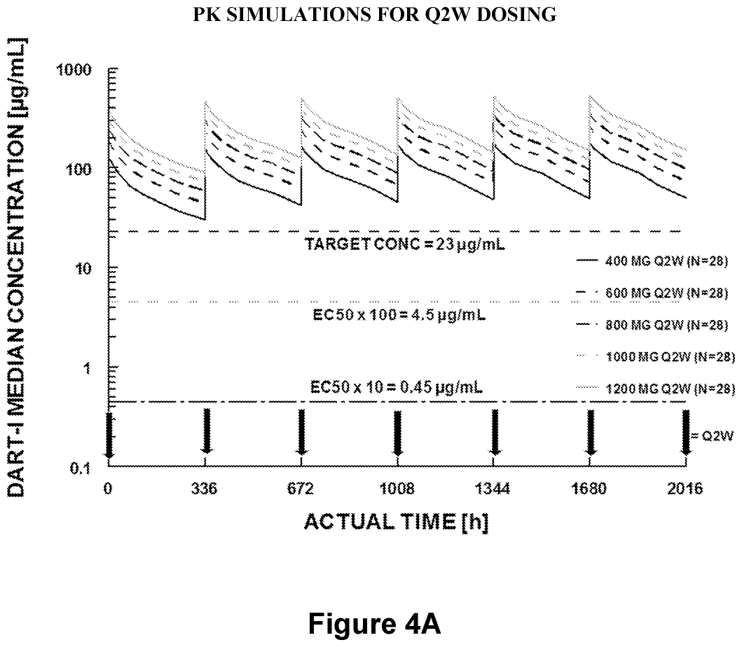

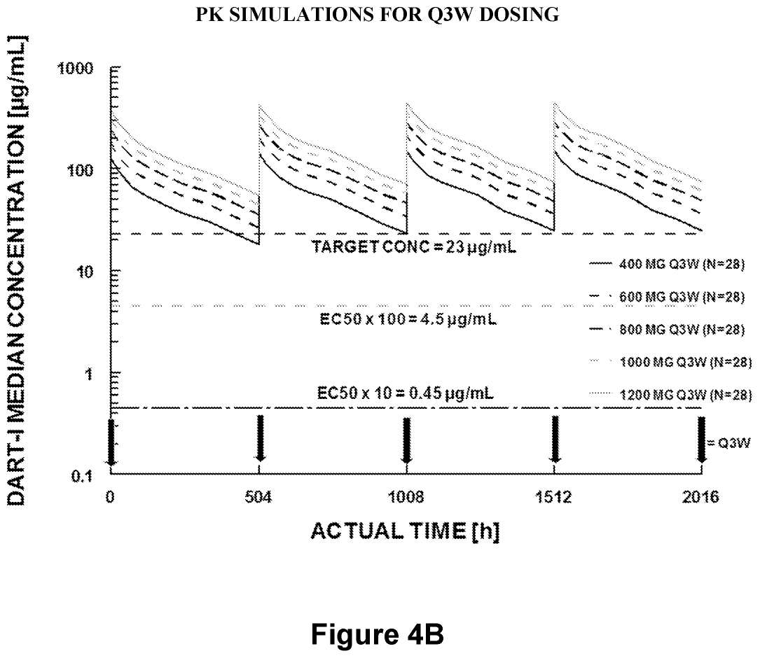

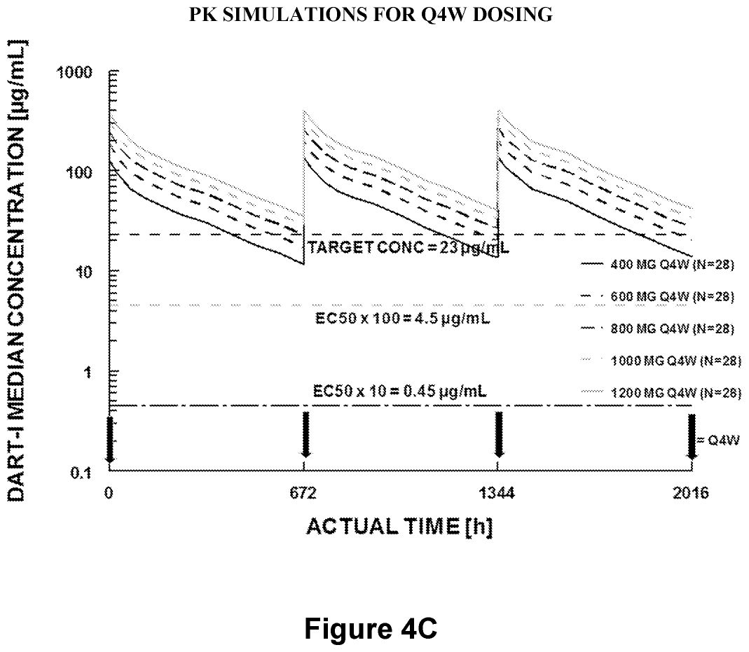

Additional PK simulations based on the serum concentration data of the patients dosed with dosing regimens of 400 mg to 1200 mg Q2W (n=28) (see above for details regarding data analysis and modeling) were performed for 1-compartment: V and CL, and for 2-compartment: V1, V2, CL and CLD. Simulated multiple dose median PK profiles for 400, 600, 800, 1000, and 1200 mg doses using Q2W, Q3W, and Q4W (once every four weeks) regimens are depicted in FIGS. 4A, 4B, and 4C, respectively. As shown in FIGS. 4A-4C, the median PK profiles indicated that DART-I target trough concentrations (C_{trough}) of ≥23 μg/mL may be obtained with administration of ≥400 mg using the Q2W, and with administration of ≥600 mg DART-I using Q3W regimen. In addition, all simulated DART-I doses and regimens result in DART-I C_{tough}≥100×RO EC₅₀ of 4.5 μg/mL.

These studies support a number of doses and regimens to attain the target threshold trough concentration (23 μg/mL). These studies support the effectiveness of a dosing regimen that comprises the administration of greater than or equal to about 400 mg of a PD-1×LAG-3 bispecific molecule of the present invention Q2W, and particularly the effectiveness of a dosing regimen that comprises the administration of about 600 mg of a PD-1×LAG-3 bispecific molecule of the present invention Q2W. These studies also particularly support the effectiveness of a dosing regimen that comprises the administration of greater than or equal to about 600 mg of a PD-1×LAG-3 bispecific molecule of the present invention Q3W. Additionally, as noted above, maximum RO was observed at doses ≥120 mg over the entire Q2W dosing regimen. Thus, these studies support the effectiveness of a dosing regimen that comprises administration of ≥about 120 mg Q2W to provide a target trough concentration of a PD-1×LAG-3 bispecific molecule of the present invention sufficient to achieve a maximum RO.

### Summary of Initial Clinical Findings

The findings after treatment of an initial 188 patients (47 patients (49% checkpoint-experienced) in Q2W Dose Escalation, and of a subsequent 141 patients (33% checkpoint-experienced) in the Q2W Cohort Expansion) are provided. Treatment-related adverse events (TRAEs) occurred in 117/188 (62.2%) patients, most commonly fatigue (n=33) and nausea (n=20). The rate of Grade ≥3 TRAEs was 19.7%. Immune-related adverse events were consistent with events observed with anti-PD-1 antibodies. Mean half-life was approximately 11 days; peripheral blood flow cytometry analyses confirmed full and sustained on-target binding during treatment at doses ≥120 mg.

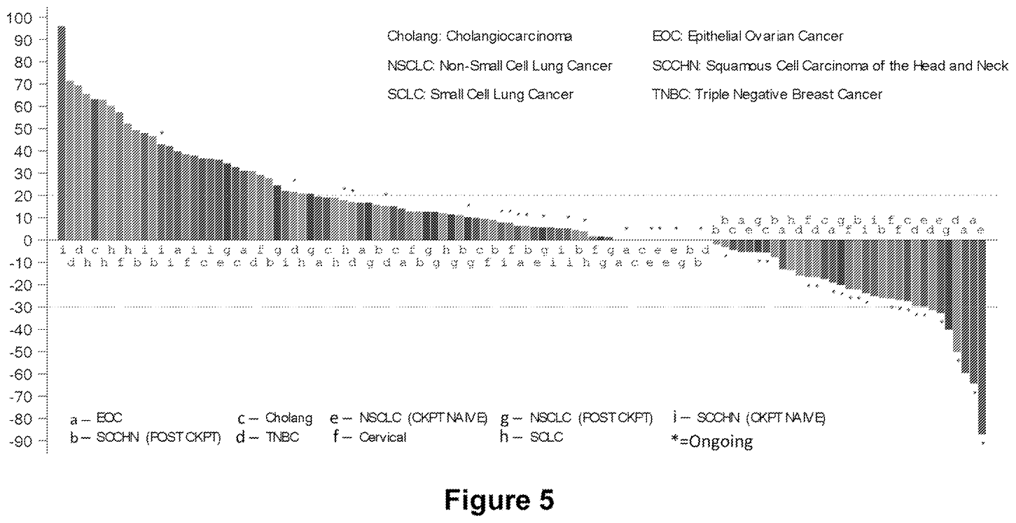

Among the first 39 response-evaluable Dose Escalation patients treated with DART-I monotherapy at doses ranging from 1 to 1200 mg Q2W, 3 confirmed partial responses (PRs) in patients having triple negative breast cancer (TNBC), mesothelioma, or gastric cancer) per RECIST 1.1 were observed, while 19 patients had stable disease. Although the study is ongoing and data is maturing, FIG. 5 presents a waterfall plot demonstrating the percent of reduction of target lesions among 120 response-evaluable cohort expansion patients receiving DART-I monotherapy at 600 mg Q2W. Among the monotherapy solid tumor expansion cohorts (i.e. excluding diffuse large B-cell lymphoma [DLBCL]), 7 objective responses per RECIST 1.1 have been observed to date (3 confirmed/4 unconfirmed), including 6 PRs (ovarian, NSCLC, and TNBC [n=2 each] and 1 complete response [CRs] (NSCLC). 51 patients had stable disease. Further results from 75 response evaluable patients in the TNBC, EOC, NSCLC (checkpoint inhibitor (CPI) naïve and post prior PD-1 checkpoint) expansion cohorts are summarized in Table 11.

TABLE 11
Summary of Response Rates (75 evaluable patients) - Monotherapy

| | | | NSCLC | NSCLC |
|---|---|---|---|---|
| | TNBC | EOC | CPI-Naïve | post-PD1 |
| Evaluable Patients | 23 | 23 | 14 | 15 |
| ORR (confirmed) | 4.3% (1/23) | 8.7% (2/23) | 14.3% (2/14) | 0% (0/15 |
| ORR (confirmed and | 17.4% (4/23) | 8.7% (2/23) | 21.4% (3/14) | 13.3% (2/15) |
| unconfirmed | | | | |
| SD | 34.8 (8/23) | 43.5 (10/23) | 50% (7/14) | 53.3% (8/15) |
| DCR | 39.1% (9/23) | 52.2 (12/23) | 64.3% (9/14) | 53.3% (8/15) |

Within the DLBCL expansion cohort, among 2 evaluable patients, 1 CR and 1 PR have been observed per the Lugano Classification. In particular, a DLBCL patient status-post CD19-targeted CAR T-cell relapse experienced a CR after a single DART-I infusion (600 mg). A checkpoint inhibitor naïve NSCLC patient (post lobectomy and carboplatin+pemetrexed treatment) experienced a CR after an 8-week Cycle (four administrations of DART-I 600 mg Q2W). Further results from 13 response-evaluable patients in the DLBCL expansion cohort are summarized in Table 12. In this larger group, 7 patients have responded encompassing activated B-cell (ABC), germinal center B-cell (GCB), and double-hit (MYC/BCL2) molecular subtypes. Duration of Response ranges from 1 (2nd scan data pending) to 168 days, with 6 of 7 responders remaining in response. The monotherapy generally well-tolerated among heavily pre-treated R/R DLBCL patients. Infusion related reactions manageable and there was no evidence of tumor lysis syndrome. These results demonstrate antitumor activity among CAR T-experienced and -naive R/R DLBCL patients, representing various molecular subtypes with a preliminary ORR: 53.8%

TABLE 12
Summary of Response Rates (13 evaluable patients) - Monotherapy

| | No. (%) of Response-Evaluable Patients† | | | | | |
|---|---|---|---|---|---|---|
| | Post CAR T | | CAR T Naïve | | Total | |
| Best Overall Response‡ | (N = 6) | | (N = 7) | | (N = 13) | |
| CR | 2 | (33.3) | 0 | (0) | 2 | (15.4) |
| PR | 0 | (0) | 5 | (71.4) | 5 | (38.5) |
| Stable Disease | 0 | (0) | 0 | (0) | 0 | (0) |
| Progressive Disease | 4 | (66.7) | 2 | (28.6) | 6 | (46.2) |
| ORR, n (%) | 2 | (33.3) | 5 | (71.4) | 7 | (53.8) |
| DCR, n (%) | 2 | (33.3) | 5 | (71.4) | 7 | (53.8) |

†patients treated with at least one post-baseline tumor assessment, and excludes 3 patients who discontinued treatment prior to first scan due to death (n = 2) and adverse event (n = 1)
‡tumor assessments per the Lugano classification

In a cohort of patients with HER-2+ tumors treated with DART-I in combination with an anti-HER-2 antibody (margetuximab), two HER2+ breast cancer patients have experienced partial responses (PRs), one confirmed and one unconfirmed, amongst the first 5 evaluable patients treated. In particular, a patient with heavily-pretreated breast cancer, with extensive chest wall involvement and hepatic and pulmonary metastases, experienced dramatic disease regression two weeks after a single administration of the combination, with a PR demonstrated at the first on-treatment disease assessment. In addition, objective responses have been observed in several patients after prior anti-PD-1 therapy. Additional results for the combination cohort are provided below.

Pre-treatment tumor biopsy samples were evaluated for both LAG-3 expression and PD-L1 expression. Briefly, LAG-3 expression was examined using LAG-3 Ab clone EPR4392(2) (Abcam) IHC assays on the Ventana Discovery Ultra platform. Positivity was defined as at least one LAG-3+ve tumor-infiltrating lymphocyte (TIL) per 40× magnification hot spot field (HSF). PD-L1 TPS/CPS expression was determined per Agilent PD-L1 (22C3) pharmDx kit instructions. As used herein, “−ve” denotes “negative” and “+ve” denotes “positive”.

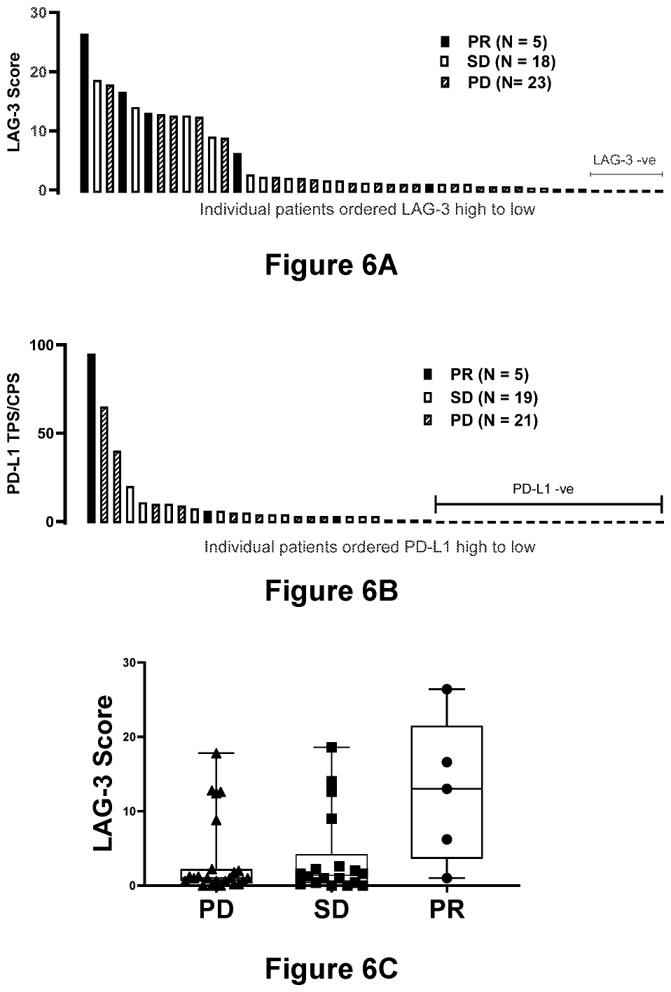

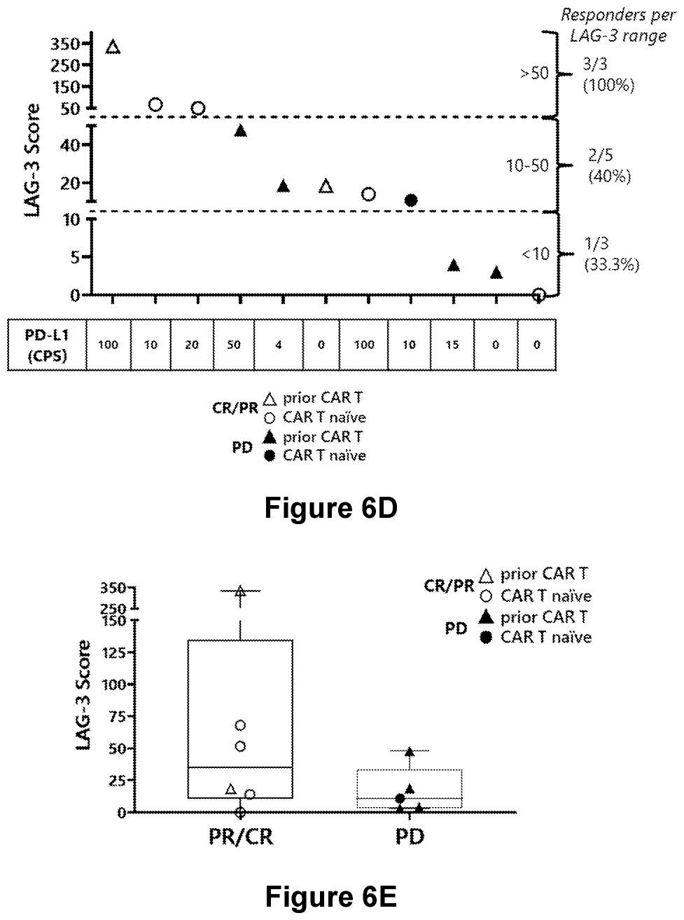

Retrospective immunohistochemistry (IHC) was performed. Briefly, Archival biopsies from TNBC, EOC, and NSCLC expansion cohorts analyzed for LAG-3 (N=46) or PD-L1 (N=45) by IHC. LAG-3 Ab clone EPR4392(2) (Abcam) IHC assays were performed on the Ventana Discovery Ultra platform. LAG-3 score was determined by calculating mean value of LAG-3+ cells per 40× field across 5 LAG-3+ hot spots. PD-L1 expression was determined per Agilent PD-L1 (22C3) pharmDx kit; TPS (NSCLC) was calculated as per interpretation manual and CPS (EOC, TNBC) calculated as follows: number of PD-L1+ cells (tumor and immune)/total number of viable tumor cells×100. CPS <1 or TPS <1% was considered negative. The individual patient LAG-3 and PD-L1 scores, with clinical responses indicated, are plotted in FIGS. 6A and 6B respectively. The LAG-3 scores plotted by clinical response are plotted in FIG. 6C.

Additional, IHC analysis was performed on biopsies obtained from the DLBCL patient (post CD19-targeted CAR T-cell relapse) who exhibited a complete response after a single dose of DART-I. Lymph node biopsy samples pre and post CAR T-cell treatment (pre-DART-I treatment) were evaluated for expression of CD3 (T-cell marker), CD79a (B-cell marker) and for PD-1 and LAG-3 by multiplex IF (fluorescence) staining with the using the HALO® image analysis platform. DAPI staining was used to determine the total cell count and the number of cells positive for each marker. The number of single, dual and triple positive cells, as a percent of the DAPI stained cells are presented in Table 13 and show that the number of cells positive for PD-1 and/or LAG-3 and/or CD3 was significantly higher post-CAR T-cell treatment. The expression of LAG-3 was the highest observed in the biopsies examined in this analysis.

TABLE 13
Summary of Single, Dual, and Triple Positive Cells

| | Pre-CAR T-cell | Post-CAR T-cell |
|---|---|---|
| | % of total | % of total |
| Staining | DAPI cells | DAPI cells |
| PD-1 + ve cells | 0.1 | 34.0 |
| LAG-3 + ve cells | 0.0 | 26.7 |
| CD3 + ve cells | 0.0 | 51.9 |
| CD79a + ve cells | 12.5 | 11.3 |
| Dual PD-1 + ve/LAG-3 + ve cells | 0.0 | 19.2 |
| Dual PD-1 + ve/CD3 + ve cells | 0.0 | 28.6 |
| Dual PD-1 + ve/CD79a + ve cells | 0.05 | 7.1 |
| Dual LAG-3 + ve/CD3 + ve cells | 0.0 | 21.2 |
| Dual LAG-3 + ve/CD79a + ve cells | 0.0 | 5.1 |
| Dual CD3 + ve/CD79a + ve cells | 0.003 | 7.9 |
| Triple CD3 + ve/LAG-3 + ve/ | 0.0 | 16.7 |
| PD-1 + ve cells | | |
| Triple CD79a + ve/PD-1 + ve/ | 0.0 | 4.5 |
| LAG-3 + ve cells | | |

Additional pre-treatment biopsies available from the DLBCL expansion cohort (N=11)) were analyzed for LAG-3 and PD-L1 expression by IHC essentially as described above. The results are shown in FIGS. 6D and 6E. FIG. 6D plots individual patients order of LAG-3 expression from high to low, with the responders per LAG-3 expression range indicated on the right. In addition, the PD-L1 score (CPS) indicated in the boxes below the plot. FIG. 6E plots the LAG-3 expression by objective response. These results indicate that DLBCL patients displaying higher baseline levels of LAG-3 appear to show improved response.

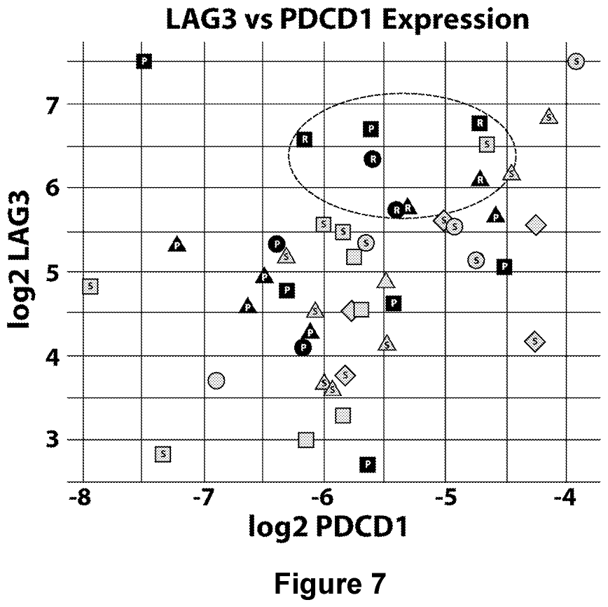

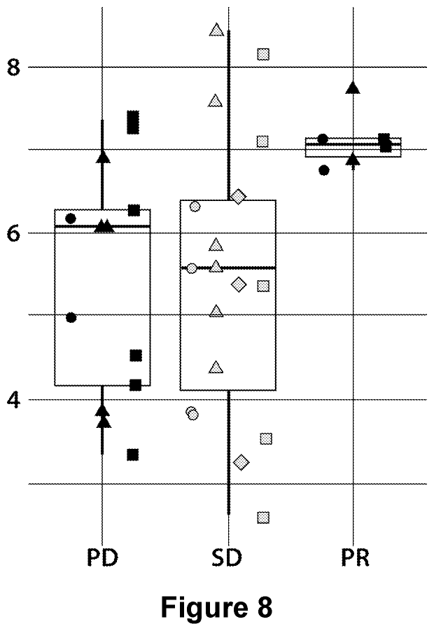

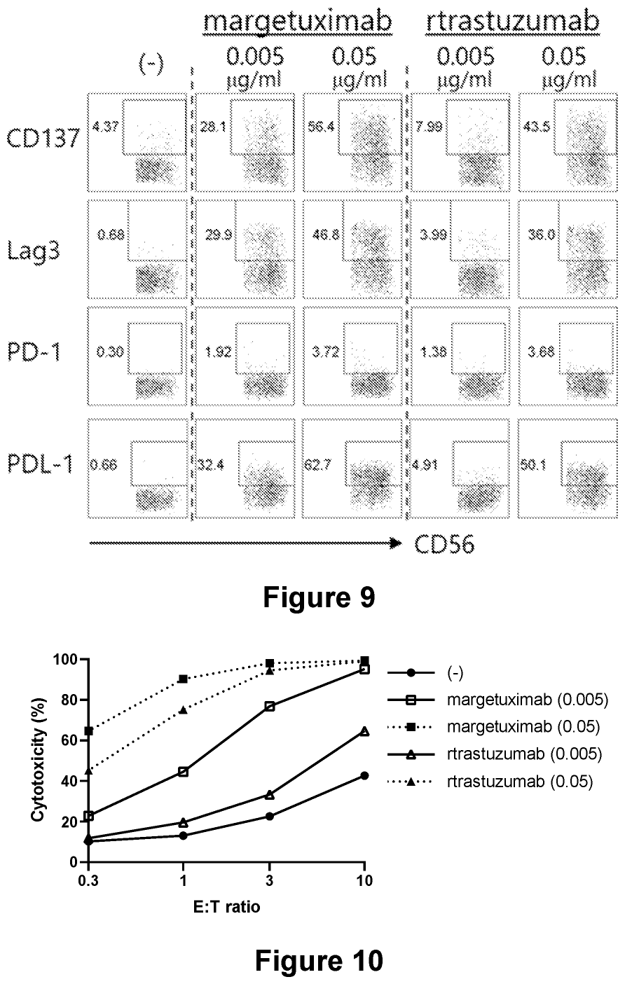

The NanoString PanCancer IO 360™ assay was used to interrogate gene expression, including the abundance of 14 immune cell types and 32 immuno-oncology signatures from archival biopsies from EOC (N=14) NSCLC (N=25, including post prior checkpoint treatment (P-NSCLC)) and TNBC (N=13) expansion cohorts. The LAG-3 vs PD-1 (PDCD1) expression is plotted in FIG. 7, and shows that the responding patients exhibit higher levels of both LAG-3 and PD-1 expression (indicated by the dotted circle). The IFN-7 Gene Signature (CXCL9, CXCL10, CXC11, STAT1) scores are plotting by clinical response are plotting in FIG. 8 and shows that patients exhibiting a partial response have higher IFN-7 Gene Signature Scores. These studies indicate that objective responses are associated with high baseline LAG-3/PD-1 expression and IFN-7 gene signature scores.

These data indicate that the PD-1×LAG-3 bispecific molecules of the present invention (illustrated by DART-I), which are designed to coordinately block PD-1 and LAG-3, demonstrated an acceptable safety profile and exhibited encouraging evidence of antitumor activity, particularly in patients having tumors that exhibit higher levels of LAG-3 expression and those having higher IFN-7 Gene Signature Scores. These data support several dosing regimens for such molecules (and of DART-I, in particular) including administration of: about ≥400 mg of such molecules (and of DART-I, in particular) Q2W (particularly about 400 mg Q2W or about 600 mg Q2W), and about 600 mg of such molecules (and of DART-I, in particular) Q3W (particularly about 600 mg Q3W or about 800 mg Q3W) to achieve a target C_{tough}≥23 μg/mL. Alternative dosing regiments include: about 120 mg of such molecules Q2W to achieve a target C_{trough} ≥than 100×RO EC₅₀. These studies further support the administration of a PD-1×LAG-3 bispecific molecule of the present invention according to any of the above doses and regimens in combination with a TA-Binding Molecule, and particularly a HER2-Binding Molecule (e.g., an anti-HER2 antibody) for the treatment of HER2 expressing (HER2+) cancers. In particular, the administration of about ≥600 mg of such molecule (and of DART-I, in particular) using a Q3W regimen in combination with a TA-Binding Molecule such as a HER2-Binding Molecule that may also administered using a Q3W regimen (e.g., margetuximab administered at 15 mg/kg Q3W).

### Example 2

### TA-Binding Molecule Mediated Changes in Checkpoint Expression and NK Cell Activity

The ability of TA-Binding Molecules comprising ADCC-Enhanced Fc Domains and wild-type Fc Domains to mediate changes in expression of checkpoint molecules on the surface of immune effectors cells, particularly NK cells was evaluated in vitro. In addition, the impact on in vitro cytotoxic activity (particularly NK cell cytotoxic activity) was examined. Briefly, PBMC effector cells (0.5×10⁶/ml) were co-incubated with N87 target cells (0.05×10⁶/ml)(E:T ratio was 10:1) that are positive for the TA HER2 (HER2⁺⁺⁺ gastric cancer cell line) in the presence of margetuximab (a TA-Binding Molecule that binds an epitope of HER2 and comprises an ADCC-Enhanced Fc Domain, i.e., an ADCC-Enhanced TA-Binding Molecule), a replica of trastuzumab (“rtrastuzumab” which binds the same epitope of HER2 but comprises a wild-type Fc Domain), or PBS (Phosphate-buffered saline) alone. The antibodies were used at 0.005 μg/ml and 0.05 μg/ml and 20 u/ml IL-2 was added to the culture. RPMI 1640 medium with L-glutamine supplemented with 10% FBS, 10 mM HEPEs buffer, and penicillin-streptomycin was used as culture medium.

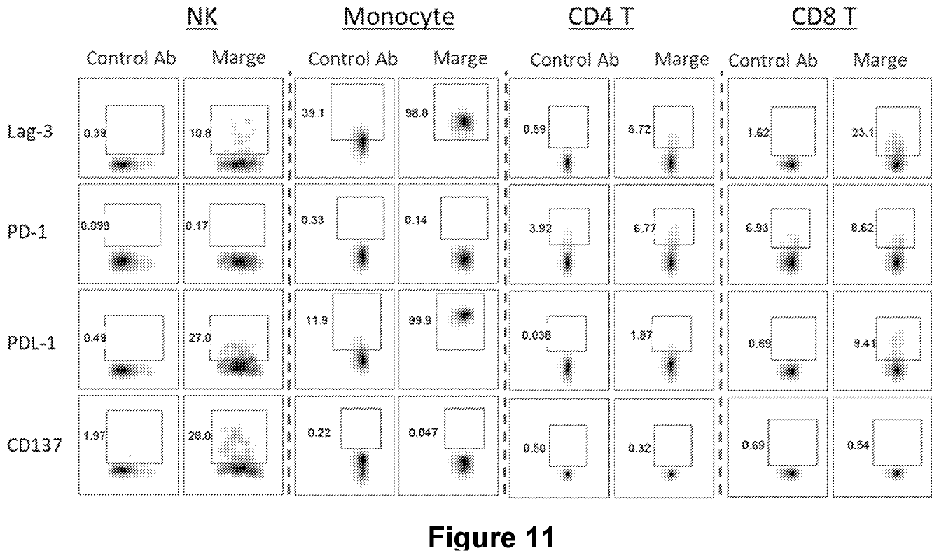

On day 3 a portion of each sample was removed and the cell surface expression of the checkpoint proteins: CD137, LAG-3, PD-1, and PD-L1 on NK cells was examined by fluorescence activated cell sorting (FACS). The following Abs were used to define immune cell subsets and the expression of cell surface checkpoint proteins: CD3-V500, CD4-PerCP Cy5.5, CD8-FITC, CD56-PE, Lag-3-PE-Cy7, PDL-1-APC, CD137-BV421, PD-1-BV650. Cell surface staining was performed by incubating cells with cocktail of Abs for 30 minutes at 4° C. in FACS buffer followed by washing with PBS, then labeled cells were resuspended in FACS buffer. FACS samples were acquired using a LSRFortessa flow cytometer and analyzed using FlowJo software. Representative FACS plots are shown in FIG. 11 with the checkpoint positive NK cells boxed and the percent indicated. As seen in FIG. 11 margetuximab up-regulated the expression of CD137, LAG-3 and PD-L1 to a greater extent than rtrastuzumab.

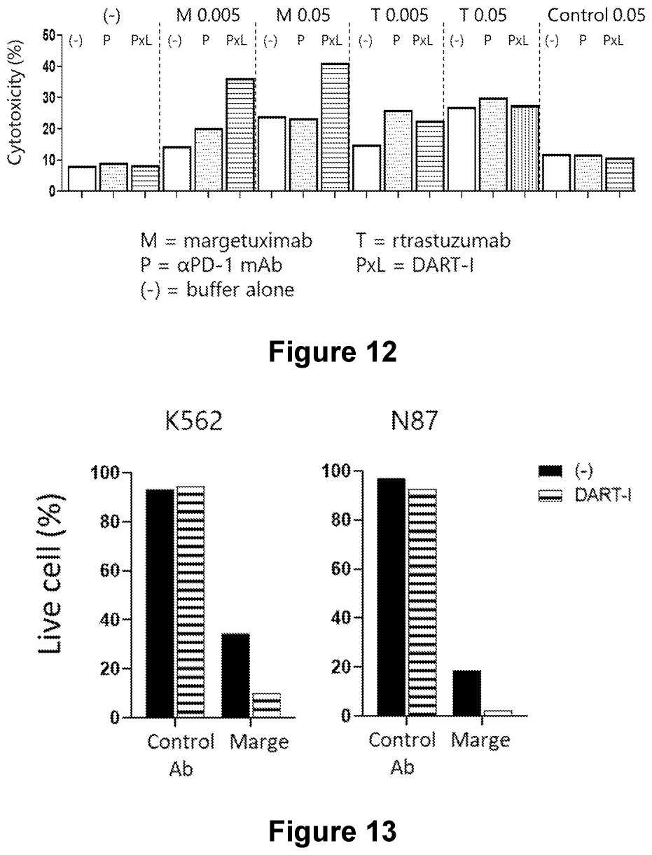

On day 6 a portion of the remaining sample was used to supply effector cells for a cytotoxicity assay using PKH26 red labeled K562 cells (a HER2-, myelogenous leukemia cell line) as target cells at E:T ratios of 0.3:1, 1:1, 3:1 and 10:1. After 4-hour incubation, cells were collected and cytotoxicity was determined by FACS analysis of 7-AAD and Annexin V as markers to distinguish live, apoptotic, and dead cells according to manufacturer's instructions. The percent cytotoxicity observed at each E:T ratio is plotted in FIG. 12. As the K562 target cells do not express HER2 the killing in this assay is not directly mediated by the binding of anti-HER2 antibodies to the K562 target cells, but rather reflects the enhancement of cytotoxic activity (primarily NK cells) mediated by the prior exposure the anti-HER2 antibodies in the presence of TA positive target cells. As shown in FIG. 12, the margetuximab mediates a stronger enhancement of NK cell cytotoxic activity as compared to rtrastuzumb. These results indicate that TA-Binding Molecules comprising ADCC-Enhanced Fc Domains are more potent mediators of PD-L1 and LAG-3 expression, and cytotoxic activity (primarily NK cells).

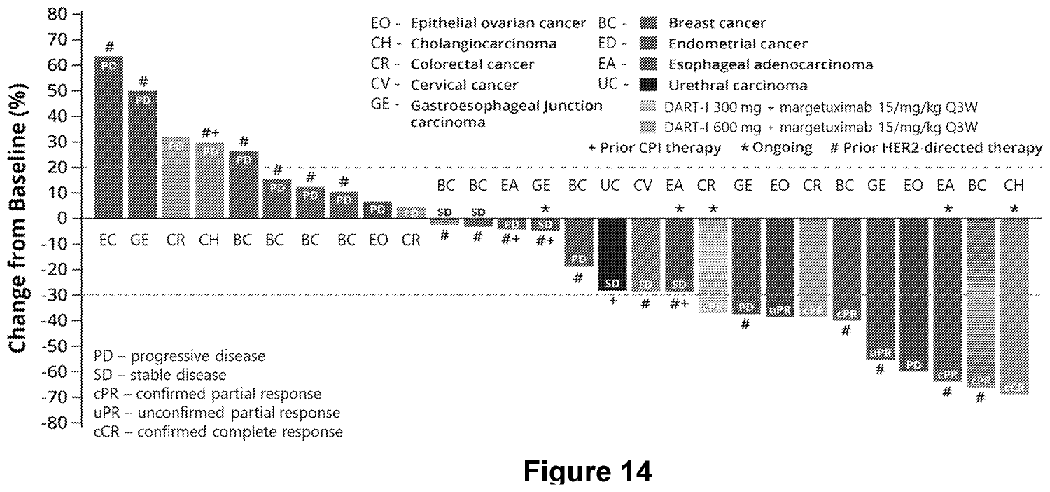

The ability of ADCC-Enhanced TA-Binding Molecules to mediate changes in expression of checkpoint molecules on the surface of additional immune cell types was examined. Briefly, PBMC effector cells (1.5×10⁶/ml) were co-incubated with N87 target cells (HER2⁺⁺⁺ gastric cancer cell line) at 15:1 E:T ratio in the presence of margetuximab (0.5 μg/ml), or a control antibody (MGAWN1, 0.5 μg/ml). RPMI 1640 medium with L-glutamine supplemented with 10% FBS, 10 mM HEPEs buffer, and penicillin-streptomycin was used as culture medium. On days 2 and 3 the cell surface expression of the checkpoint proteins: CD137, LAG-3, PD-1, and PD-L1 on NK cells (Day 3), Monocytes (Day 2), CD4⁺ (Day 3), and CD8⁺ T cells (Day 3) was examined by FACS. The following antibodies (Abs) were used to define immune cell subsets and the expression of cell surface checkpoint proteins: CD3-V500, CD4-PerCP Cy5.5, CD8-FITC, CD14-FITC, CD56-PE, Lag-3-PE-Cy7, PDL-I-APC, CD137-BV421, PD-1-BV650. Cell surface staining was performed by incubating cells with cocktail of Abs for 30 minutes at 4° C. in FACS buffer followed by washing with PBS, then labeled cells were resuspended in FACS buffer. FACS samples were acquired using a LSRFortessa flow cytometer and analyzed using FlowJo software.. Representative FACS plots are shown in FIG. 13 with the checkpoint positive immune cells boxed and the percent indicated. As seen in FIG. 13 the ADCC-Enhanced TA-Binding Molecule margetuximab mediated the up-regulation of LAG-3 and PD-L1 expression on all of the cell types examined, with most prominent up-regulation observed on monocytes, NK-cells and CD8 T-cells. CD137 was upregulated on NKs, and PD-1 was upregulated on both CD4⁺, and CD8⁺ T cells.

### Example 3

### In-Vitro Combination Studies

As described above, TA-Binding Molecules generally, and particularly those having ADCC-Enhanced Fc Domains were found to potently mediate the upregulation of the checkpoint molecules PD-L1 and LAG-3. The activity of a TA-Binding Molecule in combination with checkpoint inhibitor, that blocks LAG-3 and/or PD-1/PD-L1 inhibitory checkpoint pathways, was examined in vitro. Briefly, PBMC effector cells (0.5×10⁶/ml) were co-incubated with N87 target cells (HER2⁺⁺⁺ gastric cancer cell line) at 20:1 ratio in the presence of the TA-Binding Molecule margetuximab (comprising an ADCC-Enhanced Fc Domain), or rtrastuzumab (comprising a wild-type Fc Domain), a control antibody (MGAWN1, anti-WNV mAb comprising a wild-type human IgG1 Fc domain), or PBS, alone, or in combination with retifanlimab (a PD-1-Binding Molecule), DART-I (a bispecific molecule that binds both PD-1 and LAG-3). The assays were done with or without exogenous IL-2 (20 u/ml) (representing optimal and suboptimal conditions). The anti-HER2 antibodies were used at 0.005 μg/ml and/or 0.05 μg/ml, the anti-PD-1 antibody retifanlimab was used at 5 μg/ml, the PD-1×LAG-3 bispecific molecule DART-I was used at 5 μg/ml. On day 6, cells were collected as effectors and the cytotoxicity towards K562 target cells (E:T=10:1) was determined by FACS using 7-AAD and Annexin V as markers to distinguish live, apoptotic, and dead cells, essential as described above. The percent cytotoxicity observed for the suboptimal condition from a representative donor is plotted in FIG. 14.

As shown in FIG. 14, in this assay minimal enhancement in cytotoxicity was observed with rtrastuzumab in combination with the PD-1 checkpoint inhibitor, retifanlimab, or with the PD-1×LAG-3 dual checkpoint inhibitor DART-I. In contrast, the PD-1×LAG-3 dual checkpoint inhibitor DART-I enhanced cytotoxicity in combination with the TA-Binding Molecule having an ADCC-Enhanced Fc Domain, margetuximab. In some donors, the PD-1 checkpoint inhibitor, retinfanlimab, was also seen to enhance the cytotoxicity of margetuximab.

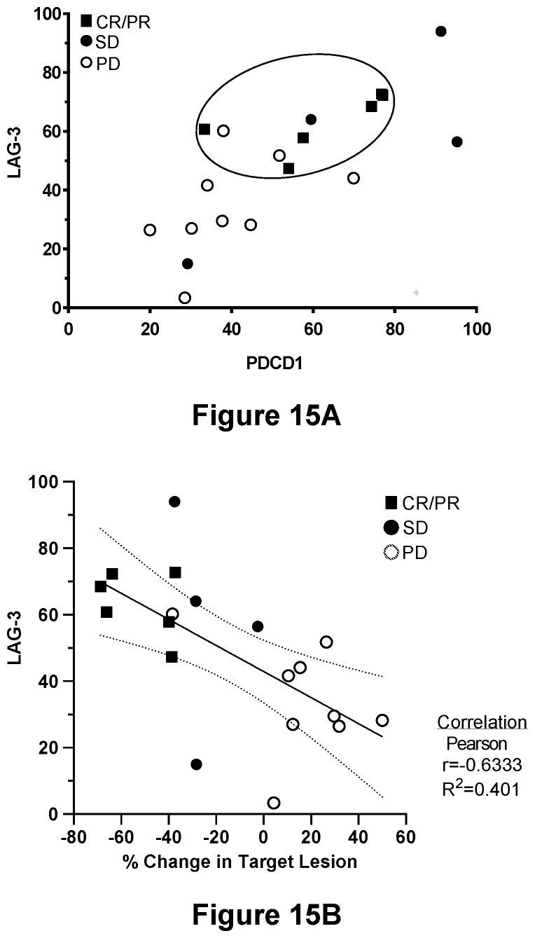

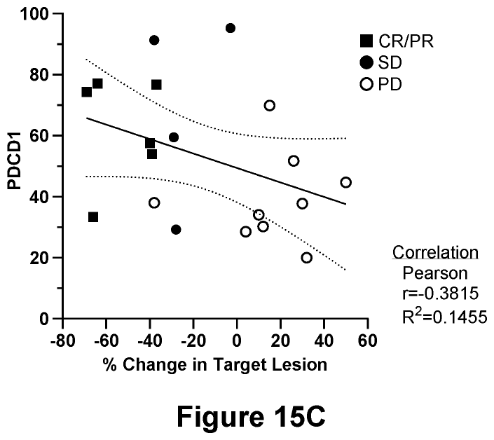

In another study the activity of the ADCC-Enhanced TA-Binding Molecule margetuximab, alone or in combination with the PD-1×LAG-3 dual checkpoint inhibitor DART-I was further examined. Briefly, PBMC effector cells (1×10⁶/ml) were co-incubated with N87 target cells (HER2⁺⁺⁺ gastric cancer cell line) at 15:1 ratio in the presence of the TA-Binding Molecule margetuximab (0.005 μg/ml), or a control antibody (MGAWN1, 0.005 μg/ml) alone, or in combination with DART-I(5 μg/ml). The assays were done with or without exogenous IL-2 (20 u/ml) (representing optimal and suboptimal conditions). On day 7, cells were collected as effectors and the cytotoxicity towards PKH26 red labeled K562 target cells (E:T=10:1) was determined by FACS using 7-AAD and Annexin V as markers to distinguish live, apoptotic, and dead cells, according to manufacturer's instructions. The cytotoxicity towards luciferase expressing N87 cells (E:T=3:1) was determined by evaluating the remaining cellular luciferase activity using Steady-Glo Luciferase Assay System (Promega). The percent cytotoxicity observed for the suboptimal condition from a representative donor is plotted in FIG. 15. As shown in FIG. 15, the PBMCs conditioned with the ADCC-Enhanced TA-Binding Molecule margetuximab exhibited higher cytotoxic activity (primarily NK cells) towards both K562 and HER2+ N87 cells opsonized with margetuximab compared with PBMCs conditioned with control Ab. As seen above, the PD-1×LAG-3 bispecific molecule, DART-I enhanced cytotoxicity in combination with the ADCC-Enhanced TA-Binding Molecule margetuximab. Together these studies indicate that dual checkpoint inhibition of the PD/PD-L1 and LAG-3 checkpoint pathways can synergize with the anti-tumor activity of a TA-Binding Molecule (particularly one having enhanced ADCC activity).

### Example 4

### Phase I Clinical Studies—HER2+ Arm

As described above, in the ongoing Phase I clinical study cohorts of patients with advanced or metastatic HER2+ solid tumors (particularly HER2+ gastric or breast cancer) patients receive DART-I (a bispecific molecule that binds PD-1 and LAG-3) and margetuximab (a TA-Binding Molecule that binds HER2 and has an ADCC-Enhanced Fc Domain).

The clinical results in 28 evaluable patients (includes the first 5 patients described above) with HER2+ solid tumors are summarized in FIG. 16. The objective response rate (ORR) (including patients with unconfirmed objective responses) was 28.6% (8/28), with a disease control rate of 50% (14/28). Table 14 summarizes the response rates among these patients by cancer type. The ORR of 28.65% is compares favorably to the PANACEA study (Loi, et al. 2019 Lancet Oncol. March; 20(3):371-382. doi: 10.1016/S1470-2045(18)30812-X.) with a reported ORR of 11.5% (n=52) in a single arm, multicenter Ph. 1b/2 trial of pembrolizumab+ trastuzumab in HER2+ mBC (15% ORR in PD-L1 positive (n=6/40); and 0% ORR in PD-L1 negative (n=0/12). Treatment was well-tolerated with responding patients remaining on therapy and further enrollment in HER2+ tumor-specific cohorts is ongoing.

TABLE 14
Summary of Response Rates (28 evaluable patients) - Combo Therapy

| | | GEJ | | | |
|---|---|---|---|---|---|
| | Breast | Esophageal | Colorectal | Other | Total |
| Evaluable | 9 | 7 | 4 | 8 | 28 |
| Patients | | | | | |
| ORR | 22.2% (2/9) | 14.3% (1/7) | 50% (2/4) | 12.5% (1/8) | 21.4% (6/28) |
| (confirmed) | | | | | |
| ORR | 22.2% (2/9) | 28.6% (2/7) | 50% (2/4) | 25% (2/8) | 28.6% (8/28) |
| (confirmed + | | | | | |
| unconfirmed) | | | | | |
| Disease | 44.4% (4/9) | 57.1% (4/7) | 50% (2/4) | 50% (4/8) | 50% (14/28) |
| control rate | | | | | |

Available pre-treatment tumor biopsy samples were evaluated for both LAG-3 expression and PD-L1 expression. Briefly, LAG-3 expression was examined using LAG-3 Ab clone EPR4392(2) (Abcam) IHC assays on the Ventana Discovery Ultra platform. Positivity was defined as at least one LAG-3+ve tumor-infiltrating lymphocyte (TIL) per 40× magnification hot spot field (HSF). PD-L1 TPS/CPS expression was determined per Agilent PD-L1 (22C3) pharmDx kit instructions. LAG-3 expression by IHC varied among the patients and was not found to correlate with response. It was observed that the majority of the responding patients had tumors that were PD-L1 negative by IHC (PD-L1 expression of <1). The high response rates among PD-L1 negative patients in this combination study utilizing PD-1 and LAG-3 dual checkpoint inhibition in combination with a TA-Binding Molecule having an ADCC-Enhanced Fc Domain are in contrast to published data (see, e.g., Loi, S. et al. (2019) “Pembrolizumab Plus Trastuzumab In Trastuzumab-Resistant, Advanced, HER2-positive Breast Cancer (PANACEA): a Single-Arm, Multicentre, Phase 1b-2 Trial,” Lancet Oncol. 20(3):371-382) indicate that response rates among HER2+ breast cancer patients treated with trastuzumab plus anti-PD-1 or anti-PD-L1 antibodies are 0% among PD-L1 negative patients and only 15% among PD-L1 positive patients. The high response likely reflects the synergistic activity of combining an ADCC-Enhanced TA-Binding Molecule with dual checkpoint inhibition of the PD/PD-L1 and LAG-3 checkpoint pathways.

The NanoString PanCancer IO 360™ assay was used to interrogate gene expression, including the abundance of 14 immune cell types and 32 immuno-oncology signatures, from archival biopsies of 19 HER2+ advanced solid tumor cohorts treated with margetuximab and DART-I. Normalized expression scores (standardized 0-100) for LAG-3 were plotted against PDCD1 (FIG. 17A). Correlation of standardized LAG-3 and PDCD1 expression levels to best percent change in target lesions from baseline, respectively are plotted in FIGS. 17B, and 17C, respectively. The gene expression analysis indicate that patients demonstrating objective responses exhibit higher expression of both LAG-3 and PDCD1 mRNA in baseline biopsy samples.

In this clinical trial cohort, the dual checkpoint inhibitor DART-I, in combination with the ADCC-enhanced TA-Binding Molecule margetuximab was generally well tolerated with safety profile consistent with DART-I monotherapy. Evidence of antitumor activity was observed among refractory patients with various tumor types expressing the HER2 tumor antigen (i.e., HER2+ tumors). Baseline LAG-3 and PD-1 mRNA expression appears to associate with clinical response, but the majority of responding patients with baseline PD-L1 expression of ≤1 (by IHC).

In summary, dual checkpoint inhibition of the PD-1/PD-L1 and LAG-3 checkpoint pathways with a molecule such as DART-I can synergize with the anti-tumor activity of a TA-Binding Molecule (particularly one having enhanced ADCC activity like margetuximab). Such combinations appear to be more effective than treatment with a TA-Binding Molecule alone or in combination with checkpoint inhibition of only the PD-1/PD-L1 pathway, and are useful for the treatment of PD-L1 negative patients.

All publications and patents mentioned in this specification are herein incorporated by reference to the same extent as if each individual publication or patent application was specifically and individually indicated to be incorporated by reference in its entirety. While the invention has been described in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth.

1. A method of treating a cancer comprising administering;
(a) a bispecific molecule that immunospecifically binds both PD-1 and LAG-3 (PD-1×LAG-3 bispecific molecule); or
(b) a molecule that immunospecifically binds PD-1 (PD-1-Binding Molecule) in combination with a molecule that immunospecifically binds LAG-3 (LAG-3-Binding Molecule); or
(c) a bispecific molecule that immunospecifically binds both PD-L1 and LAG-3 (PD-L1×LAG-3 bispecific molecule); or
(d) a molecule that immunospecifically binds PD-L1 (PD-L1-Binding Molecule) in combination with a LAG-3-Binding Molecule;
to a subject in need thereof.

2. The method of claim 1, wherein said cancer is characterized by the expression of a Tumor Antigen (TA), and wherein said method further comprises administering to said subject a Tumor Antigen (TA) Binding Molecule (TA-Binding Molecule).

3. (canceled)

4. The method of claim 2, wherein said TA-Binding Molecule is an antibody or comprises an ADCC-Enhanced Fc Domain.

5-6. (canceled)

7. The method of claim 1, wherein said PD-1-Binding Molecule is an antibody, said PD-L1-Binding Molecule is an antibody, and said LAG-3-Binding Molecule is an antibody.

8. The method of claim 1, wherein said method comprises administering said PD-1×LAG-3 bispecific molecule, or said PD-L1×LAG-3 bispecific molecule, to said subject at a flat dose of from about 120 mg to about 800 mg.

9. (canceled)

10. The method of claim 4, wherein said ADCC-Enhanced Fc Domain comprises:
(A) an engineered glycoform that is a complex N-glycoside-linked sugar chain that does not contain fucose, and/or that comprises a bisecting 0-GlcNAc; and/or
(B) comprises an amino acid substitution is selected from the group consisting of:
(a) one substitution selected from the group consisting of:
F243L, R292P, Y300L, V305I, I332E, and P396L;
(b) two substitutions selected from the group consisting of:
(1) F243L and P396L;
(2) F243L and R292P;
(3) R292P and V305I; and
(4) S239D and I332E;
(c) three substitutions selected from the group consisting of:
(1) F243L, R292P and Y300L;
(2) F243L, R292P and V305I;
(3) F243L, R292P and P396L; and
(4) R292P, V305I and P396L;
(d) four substitutions selected from the group consisting of:
(1) F243L, R292P, Y300L and P396L; and
(2) F243L, R292P, V305I and P396L; or
(e) five substitutions selected from the group consisting of:
(1) F243L, R292P, Y300L, V305I and P396L; and
(2) L235V, F243L, R292P, Y300L and P396L,
wherein the numbering is that of the EU index as in Kabat.

11-12. (canceled)

13. The method of claim 1, wherein:
(A) said PD-1-Binding Molecule is an antibody that comprises:
(a) a PD-1 VL Domain that comprises the amino acid sequence of SEQ ID NO:35, and a PD-1 VH Domain that comprises the amino acid sequence of SEQ ID NO:39;
(b) a VH and VL Domain of an anti-PD-1 antibody selected from Table 1; or
(c) a light chain and a heavy chain of an anti-PD-1 antibody selected from Table 1;
(B) said PD-L1-Binding Molecule is an antibody that comprises:
(a) a PD-L1 VL Domain that comprises the amino acid sequence of SEQ ID NO:43, and a PD-L1 VH Domain that comprises the amino acid sequence of SEQ ID NO:47;
(b) a VH and VL Domain of an anti-PD-L1 antibody selected from Table 2; or
(c) a light chain and a heavy chain of an anti-PD-L1 antibody selected from Table 2; and
(C) said LAG-3-Binding Molecule is an antibody that comprises:
(a) a LAG-3 VL Domain that comprises the amino acid sequence of SEQ ID NO:51, and a LAG-3 VH Domain that comprises the amino acid sequence of SEQ ID NO:55;
(b) a VH and VL Domain of an anti-LAG-3 antibody selected from Table 3; or
(c) a light chain and heavy chain of an anti-LAG-3 antibody selected from Table 3.

14-18. (canceled)

19. The method of claim 1, wherein said method comprises administering:
(1) a PD-1×LAG-3 bispecific molecule that comprises an Fc Region and a Hinge Domain; or
(2) a PD-L1×LAG-3 bispecific molecule that comprises an Fc Region and a Hinge Domain;
to said subject, wherein said Fc Region is a variant Fc Region that comprises:
(a) one or more amino acid modifications that reduces the affinity of the variant Fc Region for an FcγR; and/or
(b) one or more amino acid modifications that enhances the serum half-life of the variant Fc Region.

20. The method of claim 19, wherein said:
(a) said modifications that reduce the affinity of the variant Fc Region for an FcγR comprise the substitution of;
L234A;
L235A; or
L234A and L235A;
and
(b) said modifications that enhances the serum half-life of the variant Fc Region comprise the substitution of;
M252Y;
M252Y and S254T;
M252Y and T256E;
M252Y, S254T and T256E; or
K288D and H435K,
wherein said numbering is that of the EU index as in Kabat.

21. (canceled)

22. The method of claim 8, wherein said PD-1×LAG-3 bispecific molecule or said PD-L1×LAG-3 bispecific molecule is administered at a flat dose of about 300 mg or at a flat dose of about 600 mg.

23. (canceled)

24. The method of claim 8, wherein said flat dose is administered once about every 2 weeks or about once about every 3 weeks.

25-28. (canceled)

29. The method of claim 1, wherein said cancer is selected from the group consisting of: adrenal gland cancer, AIDS-associated cancer, alveolar soft part sarcoma, anal cancer, bladder cancer, bone cancer, brain and spinal cord cancer, breast cancer, carotid body tumor, cervical cancer, chondrosarcoma, chordoma, chromophobe renal cell carcinoma, clear cell carcinoma, colon cancer, colorectal cancer, desmoplastic small round cell tumor, ependymoma, endometrial cancer (including, unselected endometrial cancer, MSI-high endometrial cancer, dMMR endometrial cancer, and/or POLE exonuclease domain mutation positive endometrial cancer), Ewing's sarcoma, extraskeletal myxoid chondrosarcoma, gallbladder or bile duct cancer (including, cholangiocarcinoma bile duct cancer), gastric cancer, gastroesophageal junction (GEJ) cancer, gestational trophoblastic disease, germ cell tumor, glioblastoma, head and neck cancer, a hematological malignancy, a hepatocellular carcinoma, islet cell tumor, Kaposi's Sarcoma, kidney cancer, leukemia, liposarcoma/malignant lipomatous tumor, liver cancer, lymphoma, lung cancer, medulloblastoma, melanoma, meningioma, Merkel cell carcinoma, mesothelioma, multiple endocrine neoplasia, multiple myeloma, myelodysplastic syndrome, neuroblastoma, neuroendocrine tumors, ovarian cancer, pancreatic cancer, papillary thyroid carcinoma, parathyroid tumor, pediatric cancer, peripheral nerve sheath tumor, pharyngeal cancer, pheochromocytoma, pituitary tumor, prostate cancer, posterious uveal melanoma, renal metastatic cancer, rhabdoid tumor, rhabdomyosarcoma, sarcoma, skin cancer, a small round blue cell tumor of childhood, soft-tissue sarcoma, squamous cell cancer, stomach cancer, synovial sarcoma, testicular cancer, thymic carcinoma, thymoma, thyroid cancer, and uterine cancer.

30-31. (canceled)

32. The method of claim 2, wherein said cancer is a HER2 expressing cancer, and said TA-Binding Molecule is a HER2-Binding Molecule comprising a HER2-Binding Domain comprising a Light Chain Variable Domain (VL_{HER2}) and a Heavy Chain Variable Domain (VH_{HER2}), wherein:
(A) said Light Chain Variable Domain (VL_{HER2}) comprises the Light Chain Variable Domain of margetuximab that comprises the CDR_{L}1, CDR_{L}2 and CDR_{L}3 of SEQ ID NO:61, and said Heavy Chain Variable Domain (VH_{HER2}) comprises the Heavy Chain Variable Domain of margetuximab that comprises the CDR_{H}1, CDR_{H}2 and CDR_{H}3 of SEQ ID NO:66;
(B) said Light Chain Variable Domain (VL_{HER2}) comprises the CDR_{L}1, CDR_{L}2 and CDR_{L}3 of trastuzumab and said Heavy Chain Variable Domain (VH_{HER2}) comprises the CDR_{H}1, CDR_{H}2 and CDR_{H}3 of trastuzumab;
(C) said Light Chain Variable Domain (VL_{HER2}) comprises the CDR_{L}, CDR_{L}2 and CDR_{L}3 of pertuzumab and said Heavy Chain Variable Domain (VH_{HER2}) comprises the CDR_{H}1, CDR_{H}2 and CDR_{H}3 of pertuzumab; or
(D) said Light Chain Variable Domain (VL_{HER2}) comprises the CDR_{L}, CDR_{L}2 and CDR_{L}3 of hHER2 MAB-1 and said Heavy Chain Variable Domain (VH_{HER2}) comprises the CDR_{H}1, CDR_{H}2 and CDR_{H}3 of hHER2 MAB-1.

33. (canceled)

34. The method of claim 32, wherein said anti-HER2 antibody is margetuximab, and said method comprises administering margetuximab at a dosage of about 6 mg/kg to about 18 mg/kg once about every 3 weeks.

35-37. (canceled)

38. The method of claim 2, wherein said cancer is a B7-H3 expressing cancer, and said TA-Binding Molecule is a B7-H3-Binding Molecule comprising a B7-H3-Binding Domain that comprises a Light Chain Variable VL Domain and a Heavy Chain Variable (VH) Domain, wherein:
said VL Domain comprises the CDR_{L}1, CDR_{L}2 and CDR_{L}3 of SEQ ID NO:71, and said VH Domain comprises the CDR_{H}1, CDR_{H}2 and CDR_{H}3 of SEQ ID NO:76.

39. The method of any one of claims 2-31 or 38 claim 38, wherein said TA-Binding Molecule is enoblituzumab and said method comprises administering enoblituzumab at a dosage of about 6 mg/kg to about 18 mg/kg once about every 3 weeks.

40. (canceled)

41. The method of claim 38, wherein said B7-H3 expressing cancer is selected from the group consisting of: anal cancer, SCAC, a breast cancer, TNBC, a head and neck cancer, SCCHN, lung cancer, NSCLC, melanoma, uveal melanoma, prostate cancer, and mCRPC.

42. (canceled)

43. The method of claim 1, wherein cells expressing LAG-3 are present in a biopsy of said cancer prior to said treatment.

44. The method of claim 1, wherein cells expressing PD-1 are present in a biopsy of said cancer prior to said treatment.

45. The method of claim 1, wherein PD-L1 expression on the surface of cells of said cancer, prior to said treatment, is less than 1% as determined using a Combined Positive Score (CPS) or a Tumor Proportion Score (TPS).


SEQUENCE LISTING (80 sequences)

SEQ ID NO: 1 - protein, 107 aa, Homo sapiens [sequence omitted]

SEQ ID NO: 2 - protein, 104 aa, Homo sapiens
  MISC_FEATURE (1)..(104): Representative Human CL Lambda Domain
QPKAAPSVTLFPPSSEELQANKATLVCLISDFYPGAVTVAWKADSSPVKAGVETTPSKQSNNKYAASSYLSLTPEQWKSHRSYSCQVTHEGSTVEKTVAPTECS

SEQ ID NO: 3 - protein, 98 aa, Homo sapiens
  MISC_FEATURE (1)..(98): Representative Human IgG1 CH1 Domain
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRV

SEQ ID NO: 4 - protein, 98 aa, Homo sapiens
  MISC_FEATURE (1)..(98): Representative Human IgG2 CH1 Domain
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTV

SEQ ID NO: 5 - protein, 98 aa, Homo sapiens
  MISC_FEATURE (1)..(98): Representative Human IgG3 CH1 Domain
ASTKGPSVFPLAPCSRSTSGGTAALGCLVKDYFPEPVTVSWNSGALTSGVHTFPAVLQSSGLYSLSSVVTVPSSSLGTQTYTCNVNHKPSNTKVDKRV

SEQ ID NO: 6 - protein, 98 aa, Homo sapiens
  MISC_FEATURE (1)..(98): Representative Human IgG4 CH1 Domain
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRV

SEQ ID NO: 7 - protein, 15 aa, Homo sapiens
  MISC_FEATURE (1)..(15): Representative Human IgG1 Hinge Region
EPKSCDKTHTCPPCP

SEQ ID NO: 8 - protein, 12 aa, Homo sapiens
  MISC_FEATURE (1)..(12): Representative Human IgG2 Hinge Region
ERKCCVECPPCP

SEQ ID NO: 9 - protein, 62 aa, Homo sapiens
  MISC_FEATURE (1)..(62): Representative Human IgG3 Hinge Region
ELKTPLGDTTHTCPRCPEPKSCDTPPPCPRCPEPKSCDTPPPCPRCPEPKSCDTPPPCPRCP

SEQ ID NO: 10 - protein, 12 aa, Homo sapiens
  MISC_FEATURE (1)..(12): Representative Human IgG4 Hinge Region
ESKYGPPCPSCP

SEQ ID NO: 11 - protein, 12 aa, Artificial Sequence
  Representative Human IgG4 Hinge Region Containing Stabilizing S228P Substitution
ESKYGPPCPPCP

SEQ ID NO: 12 - protein, 217 aa, Homo sapiens
  MISC_FEATURE (1)..(217): Representative Human IgG1 CH2-CH3 Domain
  MISC_FEATURE (217)..(217): Xaa is Lysine (K) or is Absent
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGX

SEQ ID NO: 13 - protein, 216 aa, Homo sapiens
  MISC_FEATURE (1)..(216): Representative Human IgG2 CH2-CH3 Domain
  MISC_FEATURE (216)..(216): Xaa is Lysine (K) or is Absent
APPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDISVEWESNGQPENNYKTTPPMLDSDGSFFLYSKLTVDKSRWQQGNVFSCSVMHEALHNHYTQKSLSLSPGX

SEQ ID NO: 14 - protein, 217 aa, Homo sapiens
  MISC_FEATURE (1)..(217): Representative Human IgG3 CH2-CH3 Domain
  MISC_FEATURE (217)..(217): Xaa is Lysine (K) or is Absent
APELLGGPSVFLFPPKPKDTLMISRTPEVTCVVVDVSHEDPEVQFKWYVDGVEVHNAKTKPREEQYNSTFRVVSVLTVLHQDWLNGKEYKCKVSNKALPAPIEKTISKTKGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGX

SEQ ID NO: 15 - protein, 217 aa, Homo sapiens
  MISC_FEATURE (1)..(217): Representative Human IgG4 CH2-CH3 Domain
  MISC_FEATURE (217)..(217): Xaa is Lysine (K) or is Absent
APEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGX

SEQ ID NO: 16 - protein, 217 aa, Artificial Sequence
  ADCC-Enhanced “FcMT1” Variant IgG1 Fc Domain Comprising F243L, R292P, Y300L, V305I, and P396L Substitutions
  MISC_FEATURE (217)..(217): Xaa is Lysine (K) or is Absent
APELLGGPSVFLLPPKPKDTLMISRTPEVTCVVVDVSHEDPEVKFNWYVDGVEVHNAKTKPPEEQYNSTLRVVSILTVLHQDWLNGKEYKCKVSNKALPAPIEKTISKAKGQPREPQVYTLPPSRDELTKNQVSLTCLVKGFYPSDIAVEWESNGQPENNYKTTPLVLDSDGSFFLYSKLTVDKSRWQQGNVFSCSVMHEALHNHYTQKSLSLSPGX

SEQ ID NO: 17 - protein, 217 aa, Artificial Sequence
  ADCC-Enhanced “FcMT2” Variant IgG1 Fc Domain Comprising L235V, F243L, R292P, Y300L, and P396L Substitutions
  MISC_FEATURE (217)..(217): Xaa is Lysine (K) or is Absent
APELVGGPSVFLLPPKPKDTLMISRTPEVTCVVVDVSHEDPEVKFNWYVDGVEVHNAKTKPPEEQYNSTLRVVSVLTVLHQDWLNGKEYKCKVSNKALPAPIEKTISKAKGQPREPQVYTLPPSRDELTKNQVSLTCLVKGFYPSDIAVEWESNGQPENNYKTTPLVLDSDGSFFLYSKLTVDKSRWQQGNVFSCSVMHEALHNHYTQKSLSLSPGX

SEQ ID NO: 18 - protein, 217 aa, Artificial Sequence
  ADCC-Enhanced “FcMT3” Variant IgG1 Fc Domain Comprising F243L, R292P, and Y300L Substitutions
  MISC_FEATURE (217)..(217): Xaa is Lysine (K) or is Absent
APELLGGPSVFLLPPKPKDTLMISRTPEVTCVVVDVSHEDPEVKFNWYVDGVEVHNAKTKPPEEQYNSTLRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGX

SEQ ID NO: 19 - protein, 217 aa, Artificial Sequence
  CH2-CH3 Domain of Variant IgG1 Fc Domain Having Little or No ADCC Activity Comprising L234A, L235A, M252Y, S254T, and T256E Substitutions
  MISC_FEATURE (217)..(217): Xaa is Lysine (K) or is Absent
APEAAGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGX

SEQ ID NO: 20 - protein, 217 aa, Artificial Sequence
  CH2-CH3 Domain of Variant IgG4 Fc Domain Having Extended Half-Life Comprising M252Y, S254T, and T256E Substitutions
  MISC_FEATURE (217)..(217): Xaa is Lysine (K) or is Absent
APEFLGGPSVFLFPPKPKDTLYITREPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGX

SEQ ID NO: 21 - protein, 8 aa, Artificial Sequence
  Linker 1
GGGSGGGG

SEQ ID NO: 22 - protein, 6 aa, Artificial Sequence
  Linker 2
GGCGGG

SEQ ID NO: 23 - protein, 28 aa, Artificial Sequence
  Heterodimer-Promoting “E-Coil” Domain
EVAALEKEVAALEKEVAALEKEVAALEK

SEQ ID NO: 24 - protein, 28 aa, Artificial Sequence
  Heterodimer-Promoting “K-Coil” Domain
KVAALKEKVAALKEKVAALKEKVAALKE

SEQ ID NO: 25 - protein, 28 aa, Artificial Sequence
  Cysteine-Containing Heterodimer-Promoting “E-Coil” Domain
EVAACEKEVAALEKEVAALEKEVAALEK

SEQ ID NO: 26 - protein, 28 aa, Artificial Sequence
  Cysteine-Containing Heterodimer-Promoting “K-Coil” Domain
KVAACKEKVAALKEKVAALKEKVAALKE

SEQ ID NO: 27 - protein, 5 aa, Artificial Sequence
  Alternative Linker 2
ASTKG

SEQ ID NO: 28 - protein, 4 aa, Artificial Sequence
  Illustrative Linker 3
GGGS

SEQ ID NO: 29 - protein, 6 aa, Artificial Sequence
  Illustrative Linker 3
LGGGSG

SEQ ID NO: 30 - protein, 6 aa, Artificial Sequence
  Illustrative Linker 3
LEPKSS

SEQ ID NO: 31 - protein, 5 aa, Artificial Sequence
  Illustrative Linker 3
APSSS

SEQ ID NO: 32 - protein, 8 aa, Artificial Sequence
  Illustrative Linker 3
APSSSPME

SEQ ID NO: 33 - protein, 10 aa, Homo sapiens
  MISC_FEATURE (1)..(10): Representative Human Hinge Region
DKTHTCPPCP

SEQ ID NO: 34 - protein, 13 aa, Artificial Sequence
  Artificial Linker
GGGDKTHTCPPCP

SEQ ID NO: 35 - protein, 111 aa, Artificial Sequence
  Light Chain Variable Domain of Humanized Antibody That Binds PD-1 (VLPD-1)
EIVLTQSPATLSLSPGERATLSCRASESVDNYGMSFMNWFQQKPGQPPKLLIHAASNQGSGVPSRFSGSGSGTDFTLTISSLEPEDFAVYFCQQSKEVPYTFGGGTKVEIK

SEQ ID NO: 36 - protein, 15 aa, Artificial Sequence
  CDRL1 of Light Chain Variable Domain of Humanized Antibody That Binds PD-1 (VLPD-1)
RASESVDNYGMSFMN

SEQ ID NO: 37 - protein, 7 aa, Artificial Sequence
  CDRL2 of Light Chain Variable Domain of Humanized Antibody That Binds PD-1 (VLPD-1)
AASNQGS

SEQ ID NO: 38 - protein, 9 aa, Artificial Sequence
  CDRL3 of Light Chain Variable Domain of Humanized Antibody That Binds PD-1 (VLPD-1)
QQSKEVPYT

SEQ ID NO: 39 - protein, 119 aa, Artificial Sequence
  Heavy Chain Variable Domain of Humanized Antibody That Binds PD-1 (VHPD-1)
QVQLVQSGAEVKKPGASVKVSCKASGYSFTSYWMNWVRQAPGQGLEWIGVIHPSDSETWLDQKFKDRVTITVDKSTSTAYMELSSLRSEDTAVYYCAREHYGTSPFAYWGQGTLVTVSS

SEQ ID NO: 40 - protein, 5 aa, Artificial Sequence
  CDRH1 of Heavy Chain Variable Domain of Humanized Antibody That Binds PD-1 (VHPD-1)
SYWMN

SEQ ID NO: 41 - protein, 17 aa, Artificial Sequence
  CDRH2 of Heavy Chain Variable Domain of Humanized Antibody That Binds PD-1 (VHPD-1)
VIHPSDSETWLDQKFKD

SEQ ID NO: 42 - protein, 10 aa, Artificial Sequence
  CDRH3 of Heavy Chain Variable Domain of Humanized Antibody That Binds PD-1 (VHPD-1)
EHYGTSPFAY

SEQ ID NO: 43 - protein, 107 aa, Artificial Sequence
  Light Chain Variable Domain of Humanized Antibody That Binds PD-L1 (VLPD-L1)
DIQMTQSPSSLSASVGDRVTITCKASQDVNTAVAWYQQKPGKAPKLLIYWASTRHTGVPSRFSGSGSGTDFTLTISSLQPEDFATYYCQQHYNTPLTFGQGTKVEIK

SEQ ID NO: 44 - protein, 11 aa, Artificial Sequence
  CDRL1 of Light Chain Variable Domain of Humanized Antibody That Binds PD-L1 (VLPD-L1)
KASQDVNTAVA

SEQ ID NO: 45 - protein, 7 aa, Artificial Sequence
  CDRL2 of Light Chain Variable Domain of Humanized Antibody That Binds PD-L1 (VLPD-L1)
WASTRHT

SEQ ID NO: 46 - protein, 9 aa, Artificial Sequence
  CDRL3 of Light Chain Variable Domain of Humanized Antibody That Binds PD-L1 (VLPD-L1)
QQHYNTPLT

SEQ ID NO: 47 - protein, 118 aa, Artificial Sequence
  Heavy Chain Variable Domain of Humanized Antibody That Binds PD-L1 (VHPD-L1)
EVQLVESGGGLVQPGGSLRLSCAASGFTFSSYTMSWVRQAPGKGLEWVAYISIGGGTTYYPDTVKGRFTISRDNAKNTLYLQMNSLKTEDTAVYYCARQGLPYYFDYWGQGTLVTVSS

SEQ ID NO: 48 - protein, 4 aa, Artificial Sequence
  CDRH1 of Heavy Chain Variable Domain of Humanized Antibody That Binds PD-L1 (VHPD-L1)
SYTM

SEQ ID NO: 49 - protein, 16 aa, Artificial Sequence
  CDRH2 of Heavy Chain Variable Domain of Humanized Antibody That Binds PD-L1 (VHPD-L1)
YISIGGGTTYYPDTVK

SEQ ID NO: 50 - protein, 9 aa, Artificial Sequence
  CDRH3 of Heavy Chain Variable Domain of Humanized Antibody That Binds PD-L1 (VHPD-L1)
QGLPYYFDY

SEQ ID NO: 51 - protein, 107 aa, Artificial Sequence
  Light Chain Variable Domain of Humanized Antibody That Binds LAG-3 (VLLAG-3)
DIQMTQSPSSLSASVGDRVTITCRASQDVSSVVAWYQQKPGKAPKLLIYSASYRYTGVPSRFSGSGSGTDFTLTISSLQPEDFATYYCQQHYSTPWTFGGGTKLEIK

SEQ ID NO: 52 - protein, 11 aa, Artificial Sequence
  CDRL1 of Light Chain Variable Domain of Humanized Antibody That Binds LAG-3 (VLLAG-3)
RASQDVSSVVA

SEQ ID NO: 53 - protein, 7 aa, Artificial Sequence
  CDRL2 of Light Chain Variable Domain of Humanized Antibody That Binds LAG-3 (VLLAG-3)
SASYRYT

SEQ ID NO: 54 - protein, 9 aa, Artificial Sequence
  CDRL3 of Light Chain Variable Domain of Humanized Antibody That Binds LAG-3 (VLLAG-3)
QQHYSTPWT

SEQ ID NO: 55 - protein, 118 aa, Artificial Sequence
  Heavy Chain Variable Domain of Humanized Antibody That Binds LAG3 (VHLAG-3)
QVQLVQSGAEVKKPGASVKVSCKASGYTFTDYNMDWVRQAPGQGLEWMGDINPDNGVTIYNQKFEGRVTMTTDTSTSTAYMELRSLRSDDTAVYYCAREADYFYFDYWGQGTTLTVSS

SEQ ID NO: 56 - protein, 5 aa, Artificial Sequence
  CDRH1 of Heavy Chain Variable Domain of Humanized Antibody That Binds LAG3 (VHLAG-3)
DYNMD

SEQ ID NO: 57 - protein, 17 aa, Artificial Sequence
  CDRH2 of Heavy Chain Variable Domain of Humanized Antibody That Binds LAG3 (VHLAG-3)
DINPDNGVTIYNQKFEG

SEQ ID NO: 58 - protein, 9 aa, Artificial Sequence
  CDRH3 of Heavy Chain Variable Domain of Humanized Antibody That Binds LAG3 (VHLAG-3)
EADYFYFDY

SEQ ID NO: 59 - protein, 496 aa, Artificial Sequence
  Amino Acid Sequence of the First and Third Polypeptide Chains of DART-I
DIQMTQSPSSLSASVGDRVTITCRASQDVSSVVAWYQQKPGKAPKLLIYSASYRYTGVPSRFSGSGSGTDFTLTISSLQPEDFATYYCQQHYSTPWTFGGGTKLEIKGGGSGGGGQVQLVQSGAEVKKPGASVKVSCKASGYSFTSYWMNWVRQAPGQGLEWIGVIHPSDSETWLDQKFKDRVTITVDKSTSTAYMELSSLRSEDTAVYYCAREHYGTSPFAYWGQGTLVTVSSGGCGGGEVAACEKEVAALEKEVAALEKEVAALEKESKYGPPCPPCPAPEFLGGPSVFLFPPKPKDTLYITREPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 60 - protein, 271 aa, Artificial Sequence
  Amino Acid Sequence of the Second and Fourth Polypeptide Chains of DART-I
EIVLTQSPATLSLSPGERATLSCRASESVDNYGMSFMNWFQQKPGQPPKLLIHAASNQGSGVPSRFSGSGSGTDFTLTISSLEPEDFAVYFCQQSKEVPYTFGGGTKVEIKGGGSGGGGQVQLVQSGAEVKKPGASVKVSCKASGYTFTDYNMDWVRQAPGQGLEWMGDINPDNGVTIYNQKFEGRVTMTTDTSTSTAYMELRSLRSDDTAVYYCAREADYFYFDYWGQGTTLTVSSGGCGGGKVAACKEKVAALKEKVAALKEKVAALKE

SEQ ID NO: 61 - protein, 107 aa, Artificial Sequence
  Light Chain Variable Domain of Margetuximab
DIVMTQSHKFMSTSVGDRVSITCKASQDVNTAVAWYQQKPGHSPKLLIYSASFRYTGVPDRFTGSRSGTDFTFTISSVQAEDLAVYYCQQHYTTPPTFGGGTKVEIK

SEQ ID NO: 62 - protein, 11 aa, Artificial Sequence
  CDRL1 of Light Chain Variable Domain of Margetuximab
KASQDVNTAVA

SEQ ID NO: 63 - protein, 7 aa, Artificial Sequence
  CDRL2 of Light Chain Variable Domain of Margetuximab
SASFRYT

SEQ ID NO: 64 - protein, 9 aa, Artificial Sequence
  CDRL3 of Light Chain Variable Domain of Margetuximab
QQHYTTPPT

SEQ ID NO: 65 - protein, 214 aa, Artificial Sequence
  Light Chain of Margetuximab
DIVMTQSHKFMSTSVGDRVSITCKASQDVNTAVAWYQQKPGHSPKLLIYSASFRYTGVPDRFTGSRSGTDFTFTISSVQAEDLAVYYCQQHYTTPPTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 66 - protein, 120 aa, Artificial Sequence
  Heavy Chain Variable Domain of Margetuximab
QVQLQQSGPELVKPGASLKLSCTASGFNIKDTYIHWVKQRPEQGLEWIGRIYPTNGYTRYDPKFQDKATITADTSSNTAYLQVSRLTSEDTAVYYCSRWGGDGFYAMDYWGQGASVTVSS

SEQ ID NO: 67 - protein, 5 aa, Artificial Sequence
  CDRH1 of Heavy Chain Variable Domain of Margetuximab
DTYIH

SEQ ID NO: 68 - protein, 17 aa, Artificial Sequence
  CDRH2 of Heavy Chain Variable Domain of Margetuximab
RIYPTNGYTRYDPKFQD

SEQ ID NO: 69 - protein, 11 aa, Artificial Sequence
  CDRH3 of Heavy Chain Variable Domain of Margetuximab
WGGDGFYAMDY

SEQ ID NO: 70 - protein, 450 aa, Artificial Sequence
  Heavy Chain of Margetuximab, Comprising the FcMT2 ADCC-Enhanced Fc Domain (Comprising L235V, F243L, R292P, Y300L, and P396L Substitutions)
QVQLQQSGPELVKPGASLKLSCTASGFNIKDTYIHWVKQRPEQGLEWIGRIYPTNGYTRYDPKFQDKATITADTSSNTAYLQVSRLTSEDTAVYYCSRWGGDGFYAMDYWGQGASVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELVGGPSVFLLPPKPKDTLMISRTPEVTCVVVDVSHEDPEVKFNWYVDGVEVHNAKTKPPEEQYNSTLRVVSVLTVLHQDWLNGKEYKCKVSNKALPAPIEKTISKAKGQPREPQVYTLPPSRDELTKNQVSLTCLVKGFYPSDIAVEWESNGQPENNYKTTPLVLDSDGSFFLYSKLTVDKSRWQQGNVFSCSVMHEALHNHYTQKSLSLSPGK

SEQ ID NO: 71 - protein, 107 aa, Artificial Sequence
  Light Chain Variable Domain of Enoblituzumab
DIQLTQSPSFLSASVGDRVTITCKASQNVDTNVAWYQQKPGKAPKALIYSASYRYSGVPSRFSGSGSGTDFTLTISSLQPEDFATYYCQQYNNYPFTFGQGTKLEIK

SEQ ID NO: 72 - protein, 11 aa, Artificial Sequence
  CDRL1 of Light Chain Variable Domain of Enoblituzumab
KASQNVDTNVA

SEQ ID NO: 73 - protein, 7 aa, Artificial Sequence
  CDRL2 of Light Chain Variable Domain of Enoblituzumab
SASYRYS

SEQ ID NO: 74 - protein, 9 aa, Artificial Sequence
  CDRL3 of Light Chain Variable Domain of Enoblituzumab
QQYNNYPFT

SEQ ID NO: 75 - protein, 214 aa, Artificial Sequence
  Light Chain of Enoblituzumab
DIQLTQSPSFLSASVGDRVTITCKASQNVDTNVAWYQQKPGKAPKALIYSASYRYSGVPSRFSGSGSGTDFTLTISSLQPEDFATYYCQQYNNYPFTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 76 - protein, 220 aa, Artificial Sequence
  Heavy Chain Variable Domain of Enoblituzumab
EVQLVESGGGLVQPGGSLRLSCAASGFTFSSFGMHWVRQAPGKGLEWVAYISSDSSAIYYADTVKGRFTISRDNAKNSLYLQMNSLRDEDTAVYYCGRGRENIYYGSRLDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRV

SEQ ID NO: 77 - protein, 5 aa, Artificial Sequence
  CDRH1 of Heavy Chain Variable Domain of Enoblituzumab
SFGMH

SEQ ID NO: 78 - protein, 17 aa, Artificial Sequence
  CDRH2 of Heavy Chain Variable Domain of Enoblituzumab
YISSDSSAIYYADTVKG

SEQ ID NO: 79 - protein, 13 aa, Artificial Sequence
  CDRH3 of Heavy Chain Variable Domain of Enoblituzumab
GRENIYYGSRLDY

SEQ ID NO: 80 - protein, 452 aa, Artificial Sequence
  Heavy Chain of Enoblituzumab Comprising the FcMT2 ADCC-Enhanced Fc Domain (Comprising L235V, F243L, R292P, Y300L, and P396L Substitutions)
EVQLVESGGGLVQPGGSLRLSCAASGFTFSSFGMHWVRQAPGKGLEWVAYISSDSSAIYYADTVKGRFTISRDNAKNSLYLQMNSLRDEDTAVYYCGRGRENIYYGSRLDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELVGGPSVFLLPPKPKDTLMISRTPEVTCVVVDVSHEDPEVKFNWYVDGVEVHNAKTKPPEEQYNSTLRVVSVLTVLHQDWLNGKEYKCKVSNKALPAPIEKTISKAKGQPREPQVYTLPPSREEMTKNQVSLTCLVKGFYPSDIAVEWESNGQPENNYKTTPLVLDSDGSFFLYSKLTVDKSRWQQGNVFSCSVMHEALHNHYTQKSLSLSPGK